(19) United States
(12) Patent Application Publication
(10) Pub. No.: US 2013/0252832 A1
(43) Pub. Date: Sep. 26, 2013

(54) KRAS Variant and Tumor Biology

(71) Applicant: Joanne B. Weidhaas, Westport, CT (US)

(72) Inventor: Joanne B. Weidhaas, Westport, CT (US)

(73) Assignee: Yale University, New Haven, CT (US)

(21) Appl. No.: 13/427,523

(22) Filed: Mar. 22, 2012

(51) Int. Cl. C40B 30/04; C12Q 1/68

(52) U.S. Cl. 506/9; 435/6.11

(57) ABSTRACT

The disclosure provides methods for identifying a subject at risk of developing cancer, predicting the onset of cancer, and predicting a subject's response to chemotherapy/treatment by determining the presence or absence of a SNP in the KRAS oncogene, known as the KRAS variant.

### GOVERNMENT SUPPORT

This invention was made, in part, with U.S. Government support under Clinical and Translational Science Awards (CTSA), grant UL1 RR024139, provided by the National Center for Research Resources, a component of the National Institutes of Health.

This invention was made, in part, with U.S. Government support under grant RO1 CA131301-01A1, provided by The National Cancer Institute, grant CA124484 (K08) provided by The National Institutes of Health, grant RO1 CA122728, provided by the National Institutes of Health, grant RO1 CA74415, provided by the National Institutes of Health, and grant RC4CA153828 provided by the National Cancer Institute and the Office of the Director of the National Institutes of Health.

The Government has certain rights in the invention.

### INCORPORATION BY REFERENCE

The contents of the text file named “34592-515001USST25.txt”, which was created on Mar. 16, 2012 and is 32.2 KB in size, are hereby incorporated by reference in their entirety.

### FIELD OF THE DISCLOSURE

This disclosure relates generally to the fields of cancer, reproductive health and molecular biology. The disclosure provides methods for diagnosing and prognosing a subject having cancer by determining the presence or absence of a genetic marker. Moreover, the disclosure provides methods for determining a subject's response to treatment by determining the presence or absence of a genetic marker.

### BACKGROUND

The heterogeneity of cancer is reflected by the variable risk factors, treatment response and outcome in patients. While prognostic gene expression markers are highly divergent, several modules such as DNA repair deficiency, signatures of immune response or epithelial-to-mesenchymal transition are commonly found to be relevant for a subset of tumors. Thus, there is a need in the art for the identification of the drivers of these transcriptional modules as a promising approach for the discovery of specific and personalized therapies.

### SUMMARY

The studies presented in this disclosure relate to a central thesis regarding the role of miRNAs in cancer: disruption of miRNAs' regulation of oncogenes or tumor suppressor genes impact cancer risk, tumor development, and response to treatment. miRNAs may regulate oncogenes or tumor suppressor genes directly or indirectly. For example, the KRAS variant, a SNP located in the let-7 complementary site 6 (LCS6) of the 3′ UTR of the KRAS gene, disrupts regulation of KRAS by the let-7 family of miRNAs. In this case, let-7-mediated regulation of KRAS is disrupted; however, there are secondary effects of the KRAS variant. Disruption of the let-7/KRAS interaction upstream perpetuates aberrant signaling to downstream factors. Furthermore, components of signaling pathways other than the canonical RAS pathway are affected. The presence of the KRAS variant increases angiogenesis, survival (even under hypoxic conditions), metastasis, and confers resistance to frequently used chemotherapy agents. Moreover, epigenetic changes in the cancer cell, such as changes to promoter methylation of tumor suppressor and cell cycle genes, influence the development, survival, and response to treatment of a cancer cell positive for the KRAS variant. Finally, the cellular consequences of the KRAS variant are independent of other mutations in KRAS, including, for example, acquired mutations in a coding region of KRAS. For many cancer cells, the occurrence of the KRAS variant is mutually exclusive with the occurrence of other KRAS mutations. Unlike acquired mutations in KRAS, the KRAS variant is a germline mutation. Thus, the KRAS variant is a heritable biomarker of tumor cell biology.

The occurrence of the KRAS variant mutation leads to increased expression and/or abundance of KRAS and decreased expression of the let-7 family of miRNAs. The KRAS variant also affects the expression levels of transcription factors and miRNAs other than let-7 family miRNAs. For example, the KRAS variant is statistically significantly associated with increased expression levels of miR-23 and miR-27, which target anti-angiogenic genes such as Sprouty 2 and Sema6A. Thus, the poor outcome and resistance to traditional chemotherapy agents may result from an ability of the KRAS variant to drive activation of cell proliferation through RAS pathways, but also angiogenesis pathways that irrigate tumors with blood and nutrients to promote survival of cancer cells within a tumor. In the face of two aberrant pathways that have a common activator, the activity of certain chemotherapeutic agents may be insufficient to combat the progression of the cancer. The perturbation of RAS and other pathways in tumors that have the KRAS variant is conserved across cancer cell and tumor types (such as breast and ovarian cancers).

The KRAS variant is associated with poor clinical outcomes in various cancers, including, but not limited to, colon, ovarian, head and neck cancer, and lung cancer. The evidence suggests that the KRAS variant determines a patient's response to treatment. If a carrier of the KRAS variant is resistant to the standard chemotherapeutic agent, then the patient's outcome is worse. The data presented herein demonstrate that the KRAS variant can confer resistance to traditional chemotherapeutic agents, while sometimes conferring increased sensitivity to monoclonal antibody therapy. For example, the KRAS variant increases a subject's sensitivity to Cetuximab when delivered as the only treatment, which targets an upstream regulator of the KRAS pathway (EGFR). Accordingly, the occurrence of the KRAS variant may suggest that agents specific for targets upstream of KRAS will be successful, however, conventional chemotherapeutic agents that target cell cycle checkpoints, which are downstream of KRAS may be ineffective. Similarly, the KRAS variant confers resistance to platinum-based chemotherapy. Platinum-based agents crosslink DNA molecules to prevent DNA replication, ultimately triggering apoptosis. However, DNA replication is a process that occurs downstream of KRAS activation, and, therefore, may be ineffective, particularly in light of data showing the recruitment of signaling pathways other than RAS.

These discoveries about KRAS tumor biology provided herein have significant clinical value because chemotherapy as a treatment method is very hard on the patient. Chemotherapeutic agents present side effects that not only add to the patient's discomfort, but also introduce complications with otherwise functioning bodily systems. For instance, a chemotherapeutic agent that kills cancer cells may also damage or weaken the patient's heart. Thus, the KRAS variant is a biomarker for determining resistance or sensitivity to known chemotherapy agents. If a patient is positive for the KRAS variant, then the doctor may be able to choose an optimal treatment, or at least avoid an ineffective treatment.

In this disclosure the terms subject and patient are used interchangeably.

The disclosure provides a method of predicting the an increased risk of vascularization of a tumor, including (a) detecting a mutation in let-7 complementary site LCS6 of human KRAS in a first patient sample, wherein the mutation is a SNP comprising a uracil (U) or thymine (T) to guanine (G) transition at position 4 of LCS6, and (b) determining the expression level of a miRNA selected from the group consisting of miR-23 and miR-27 in a second patient sample, wherein the presence of the mutation in (a) and an increase in the expression level of a miRNA in (b) compared to a control indicates increased transcriptional silencing of an anti-angiogenic gene, thereby predicting the an increased risk of vascularization of the tumor. The first and second patient samples are extracted from the same patient. Moreover, the first and second patient samples may include the same fluid, tissue, or biopsy. Preferably, the second patient sample is extracted or derived from the tumor or an area of non-tumor tissue in physical contact with the tumor (i.e., surrounding the tumor). For example, the anti-angiogenic gene can be Sprouty2 or Sema 6A. The tumor may include a cancer cell derived from a(n) AIDS-related cancer, breast cancer, cancer of the digestive/gastrointestinal tract, anal cancer, appendix cancer, bile duct cancer, colon cancer, colorectal cancer, esophageal cancer, gallbladder cancer, islet cell tumors, pancreatic neuroendocrine tumors, liver cancer, pancreatic cancer, rectal cancer, small intestine cancer, stomach (gastric) cancer, endocrine system cancer, adrenocortical carcinoma, parathyroid cancer, pheochromocytoma, pituitary tumor, thyroid cancer, eye cancer, intraocular melanoma, retinoblastoma, bladder cancer, kidney (renal cell) cancer, penile cancer, prostate cancer, transitional cell renal pelvis and ureter cancer, testicular cancer, urethral cancer, Wilms' tumor, other childhood kidney tumors, germ cell cancer, central nervous system cancer, extracranial germ cell tumor, extragonadal germ cell tumor, ovarian germ cell tumor, gynecologic cancer, cervical cancer, endometrial cancer, gestational trophoblastic tumor, ovarian epithelial cancer, uterine sarcoma, vaginal cancer, vulvar cancer, head and neck cancer, hypopharyngeal cancer, laryngeal cancer, lip and oral cavity cancer, metastatic squamous neck cancer with occult primary, mouth cancer, nasopharyngeal cancer, oropharyngeal cancer, paranasal sinus and nasal cavity cancer, pharyngeal cancer, salivary gland cancer, throat cancer, musculoskeletal cancer, bone cancer, Ewing's sarcoma, gastrointestinal stromal tumors (GIST), osteosarcoma, malignant fibrous histiocytoma of bone, rhabdomyosarcoma, soft tissue sarcoma, uterine sarcoma, neurologic cancer, brain tumor, astrocytoma, brain stem glioma, central nervous system atypical teratoid/rhabdoid tumor, central nervous system embryonal tumors, central nervous system germ cell tumor, craniopharyngioma, ependymoma, medulloblastoma, spinal cord tumor, supratentorial primitive neuroectodermal tumors and pineoblastoma, neuroblastoma, respiratory cancer, thoracic cancer, non-small cell lung cancer, small cell lung cancer, malignant mesothelioma, thymoma, thymic carcinoma, skin cancer, Kaposi's sarcoma, melanoma, or Merkel cell carcinoma. Alternatively, or in addition, the tumor or cancer is metastic.

The disclosure provides a method of predicting an increased survival or proliferation of a cancer cell under hypoxic conditions, comprising (a) detecting a mutation in let-7 complementary site LCS6 of human KRAS in a first patient sample, wherein the mutation is a SNP comprising a uracil (U) or thymine (T) to guanine (G) transition at position 4 of LCS6, and (b) determining the expression level of a miR-210 miRNA in a second patient sample, wherein the presence of the mutation in (a) and an increase in the expression level of the miRNA in (b) compared to a control predicts an increased survival or proliferation of the cancer cell under hypoxic conditions. The first and second patient samples are extracted from the same patient. Moreover, the first and second patient samples may include the same fluid, tissue, or biopsy. The cancer cell may be derived from a(n) AIDS-related cancer, breast cancer, cancer of the digestive/gastrointestinal tract, anal cancer, appendix cancer, bile duct cancer, colon cancer, colorectal cancer, esophageal cancer, gallbladder cancer, islet cell tumors, pancreatic neuroendocrine tumors, liver cancer, pancreatic cancer, rectal cancer, small intestine cancer, stomach (gastric) cancer, endocrine system cancer, adrenocortical carcinoma, parathyroid cancer, pheochromocytoma, pituitary tumor, thyroid cancer, eye cancer, intraocular melanoma, retinoblastoma, bladder cancer, kidney (renal cell) cancer, penile cancer, prostate cancer, transitional cell renal pelvis and ureter cancer, testicular cancer, urethral cancer, Wilms' tumor, other childhood kidney tumors, germ cell cancer, central nervous system cancer, extracranial germ cell tumor, extragonadal germ cell tumor, ovarian germ cell tumor, gynecologic cancer, cervical cancer, endometrial cancer, gestational trophoblastic tumor, ovarian epithelial cancer, uterine sarcoma, vaginal cancer, vulvar cancer, head and neck cancer, hypopharyngeal cancer, laryngeal cancer, lip and oral cavity cancer, metastatic squamous neck cancer with occult primary, mouth cancer, nasopharyngeal cancer, oropharyngeal cancer, paranasal sinus and nasal cavity cancer, pharyngeal cancer, salivary gland cancer, throat cancer, musculoskeletal cancer, bone cancer, Ewing's sarcoma, gastrointestinal stromal tumors (GIST), osteosarcoma, malignant fibrous histiocytoma of bone, rhabdomyosarcoma, soft tissue sarcoma, uterine sarcoma, neurologic cancer, brain tumor, astrocytoma, brain stem glioma, central nervous system atypical teratoid/rhabdoid tumor, central nervous system embryonal tumors, central nervous system germ cell tumor, craniopharyngioma, ependymoma, medulloblastoma, spinal cord tumor, supratentorial primitive neuroectodermal tumors and pineoblastoma, neuroblastoma, respiratory cancer, thoracic cancer, non-small cell lung cancer, small cell lung cancer, malignant mesothelioma, thymoma, thymic carcinoma, skin cancer, Kaposi's sarcoma, melanoma, or Merkel cell carcinoma.

The disclosure provides a method of predicting an increased survival or proliferation of a cancer cell, including (a) detecting a mutation in let-7 complementary site LCS6 of human KRAS in a first patient sample, wherein the mutation is a SNP comprising a uracil (U) or thymine (T) to guanine (G) transition at position 4 of LCS6, and (b) determining the methylation status of a promoter of a tumor suppressor gene in a second patient sample, wherein the presence of the mutation in (a) and an increase in the methylation of a promoter (b) compared to a control predicts an increased survival or proliferation of the cancer cell. The first and second patient samples are extracted from the same patient. Moreover, the first and second patient samples may include the same fluid, tissue, or biopsy. Optionally, the tumor suppressor gene is Notch1. Survival may include maintaining tumorigenic potential. The cancer cell may be derived from a(n) AIDS-related cancer, breast cancer, cancer of the digestive/gastrointestinal tract, anal cancer, appendix cancer, bile duct cancer, colon cancer, colorectal cancer, esophageal cancer, gallbladder cancer, islet cell tumors, pancreatic neuroendocrine tumors, liver cancer, pancreatic cancer, rectal cancer, small intestine cancer, stomach (gastric) cancer, endocrine system cancer, adrenocortical carcinoma, parathyroid cancer, pheochromocytoma, pituitary tumor, thyroid cancer, eye cancer, intraocular melanoma, retinoblastoma, bladder cancer, kidney (renal cell) cancer, penile cancer, prostate cancer, transitional cell renal pelvis and ureter cancer, testicular cancer, urethral cancer, Wilms' tumor, other childhood kidney tumors, germ cell cancer, central nervous system cancer, extracranial germ cell tumor, extragonadal germ cell tumor, ovarian germ cell tumor, gynecologic cancer, cervical cancer, endometrial cancer, gestational trophoblastic tumor, ovarian epithelial cancer, uterine sarcoma, vaginal cancer, vulvar cancer, head and neck cancer, hypopharyngeal cancer, laryngeal cancer, lip and oral cavity cancer, metastatic squamous neck cancer with occult primary, mouth cancer, nasopharyngeal cancer, oropharyngeal cancer, paranasal sinus and nasal cavity cancer, pharyngeal cancer, salivary gland cancer, throat cancer, musculoskeletal cancer, bone cancer, Ewing's sarcoma, gastrointestinal stromal tumors (GIST), osteosarcoma, malignant fibrous histiocytoma of bone, rhabdomyosarcoma, soft tissue sarcoma, uterine sarcoma, neurologic cancer, brain tumor, astrocytoma, brain stem glioma, central nervous system atypical teratoid/rhabdoid tumor, central nervous system embryonal tumors, central nervous system germ cell tumor, craniopharyngioma, ependymoma, medulloblastoma, spinal cord tumor, supratentorial primitive neuroectodermal tumors and pineoblastoma, neuroblastoma, respiratory cancer, thoracic cancer, non-small cell lung cancer, small cell lung cancer, malignant mesothelioma, thymoma, thymic carcinoma, skin cancer, Kaposi's sarcoma, melanoma, or Merkel cell carcinoma. Optionally, the cancer cell is a cancer stem cell.

### Breast Cancer

The disclosure provides methods for identifying a subject at risk for developing aggressive and high-risk forms of breast cancer as well as methods for predicting the onset of these forms. The data provided herein constitute the first disclosure of a mechanism delineating a detectable genomic mutation that drives the development of breast cancer tumors characterized by either a lack of expression of the estrogen receptor or the progesterone receptor. In preferred embodiments, the aggressive and high-risk form of breast cancer is triple negative breast cancer, which is further characterized by a lack of expression of the Human Epidermal growth factor Receptor 2 (HER2) gene transcript or protein.

The disclosure provides a method of identifying a subject at risk for developing an estrogen receptor (ER) and progesterone receptor (PR) negative (ER/PR negative) breast cancer, including detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of a mutation indicates greater risk of developing the ER/PR negative breast cancer.

The disclosure provides a method of predicting the onset of developing an estrogen receptor (ER) and progesterone receptor (PR) negative (ER/PR negative) breast cancer in a subject at risk for developing breast cancer, including detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of a mutation indicates an earlier onset of developing the ER/PR negative breast cancer.

In a preferred embodiment of the methods described herein, the ER/PR negative breast cancer is also negative for HER2, and therefore, is a triple negative breast cancer (TNBC). The triple negative breast cancer (TNBC) can be a basal or luminal cancer or tumor. In certain aspects of these methods, the triple negative breast cancer (TNBC) is a basal tumor that expresses a transcript or protein encoded by the epidermal growth factor receptor (EGFR) or the cytokeratin 5/6 (CK5/6) gene. In other aspects, ER/PR negative or ER/PR/HER2 negative breast cancer is further characterized by low or negative expression of the breast cancer 1 (BRCA1) gene.

The subject (patient) is preferably a pre-menopausal female; however, the subject may be of any age. Alternatively, or in addition, the subject is less than 51 years of age, however, the subject may optionally, be less than 100, 90, 80, 70, 60, 50, 40, 30, 20, or any number of years of age in between.

### Colorectal Cancer

The disclosure provides a method of prognosing a subject with colorectal cancer (CRC), including detecting a mutation in let-7 complementary site LCS6 of human KRAS in a patient sample, wherein the mutation is a SNP comprising a uracil (U) or thymine (T) to guanine (G) transition at position 4 of LCS6, wherein the presence of the KRAS-variant indicates a increased survival rate when compared to a control. In one aspect of this method, the detecting step further includes microsatellite-instability (MSI) analysis. The KRAS-variant is an independent marker of survival in colorectal cancer cells and patients; however, microsatellite instability (MSI) analysis may be used as a secondary analysis. Although MSI is a molecular marker for good prognosis in CRC patients (i.e. those with MSI tumors are considered to have a good prognosis), determination of the KRAS-variant status revealed that individuals who have developed a MSI tumor, but who are negative for the KRAS-variant (or, in other words, wild type) still have a poor prognosis in CRC. Thus, the disclosure provides a superior method for predicting the clinical outcome, or prognosis of CRC, particularly when the CRC patients are stratified by cancer stage.

In particular embodiments of this method, the colorectal cancer (CRC) is early stage CRC. Preferably, the colorectal cancer (CRC) is stage 1 or 2.

The test subject may have a second mutation in the KRAS gene, the KRAS-variant being the first mutation.

The test or control subject may carry one or more mutations in the BRAF gene. Alternatively, or in addition, the test or control subject may have a hypermethylated RASSF1A promoter.

The control subject does not carry the KRAS-variant (i.e. the control subject is wild type for the KRAS-variant mutation). However, the control subject may have CRC, or may be a cancer-free individual. Furthermore, the control subject may have a second mutation in the KRAS gene, which is not the KRAS-variant.

In certain aspects of this method, the survival rate is an overall survival rate (for instance, some examples, include, but are not limited to, survival rates calculated from the time of cancer development or diagnosis until the subject succumbs to the cancer (death), enters remission, or a doctor declares the subject cured or clean of all cancer cells), five-year survival rate or one-year survival. Shorter survival periods are calculated, for instance, from either the development or diagnosis of the cancer until a determined time, such as one or five years.

### Response to Treatment for Ovarian Cancer

The disclosure provides methods of prognosing subjects with epithelial ovarian cancer (EOC) and, furthermore, methods of optimizing treatment by predicting the subject's response to platinum-based chemotherapy. The methods and data described herein identify a specific genomic mutation in a let-7 miRNA binding site within the 3′ untranslated region (UTR) of the KRAS gene (known as the KRAS variant).

The disclosure provides a method of prognosing a subject with epithelial ovarian cancer (EOC), including detecting a mutation in let-7 complementary site LCS6 of human KRAS in a patient sample, wherein the mutation is a SNP comprising a uracil (U) or thymine (T) to guanine (G) transition at position 4 of LCS6, wherein the presence of the KRAS-variant indicates a decreased survival rate when compared to a control.

Although the method can be applied to subjects and women of all ages, in certain embodiments of this method, the test subject is post-menopausal or 52 years of age or older. Control subjects include healthy individuals and those women who have EOC, but who do not carry the KRAS-variant. Moreover, the control subject can be a national average based upon the expected survival of women born in the same year as the test subject, or who belong to the same generation as the test subject. In a preferred embodiment, this control value does not include those individuals who carry the KRAS-variant. In certain aspects of this method, the survival rate is an overall survival rate (for instance, some examples, include, but are not limited to, survival rates calculated from the time of cancer development or diagnosis until the subject succumbs to the cancer (death), enters remission, or a doctor declares the subject cured or clean of all cancer cells), five-year survival rate or one-year survival. Shorter survival periods are calculated, for instance, from either the development or diagnosis of the cancer until a determined time, such as one or five years.

The disclosure also provides a method of predicting the response of an epithelial ovarian cancer (EOC) cell to a platinum-based chemotherapy, including detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of the mutation indicates a resistance to platinum-based chemotherapy. The EOC cell may be evaluated in vitro or ex vivo. When the EOC cell is evaluated ex vivo, the cell is obtained from a subject. The subject may be of any age, however, in a preferred embodiment, the subject is either postmenopausal or at least 52 years old. Alternatively, in the same embodiment, the subject is at least 30, 35, 40, 45, 50, 55, 60, 65, 70, 75 years of age, or any age in between. In other aspects of this method, the subject is not post-menopausal, but presents a similar hormonal profile due to a second medical condition or medical treatment. An exemplary, but non-limiting menopausal hormonal profile includes decreased levels of estrogen and progesterone hormone, as determined by, for instance, assessment of a sample of the subject's blood or urine. Exemplary, but non-limiting, secondary medical conditions that induce a menopausal hormonal profile are surgical removal of at least one ovary (ovariectomy, also known as surgical menopause), cervical, uterine or ovarian cancer that necessitates a hysterectomy (especially if removal of the uterus is combined with removal of the Fallopian tubes and one or both ovaries). Exemplary, but non-limiting, secondary medical conditions that induce a menopausal hormonal profile are chemotherapy and anti-estrogen treatments.

When the EOC cell is evaluated in vitro, the cell is isolated, reproduced, or derived from the BG1, CAOV3, or IGR-OV1 cell lines. These cell lines are non-limiting examples of ovarian cancer cell lines. An EOC cell may be isolated, reproduced, or derived from any ovarian cancer cell line, including, but not limited to, those cell lines that carry the KRAS-variant, a deleterious BRCA1 mutation, a deleterious BRCA2 mutation, or any combination thereof. A deleterious BRCA1 or BRCA2 mutation is a mutation that increases the risk or likelihood that it's carrier will develop cancer, and, in preferred embodiments, breast or ovarian cancer. A deleterious BRCA1 or BRCA2 mutation is a mutation that also increases the risk or likelihood that it's carrier will develop cancer at a younger age (i.e. experience an earlier onset of cancer), and, in preferred embodiments, the cancer is breast or ovarian cancer.

For the methods described herein, the preferred platinum-based chemotherapy is carboplatin or paclitaxel, however, the platinum-based chemotherapy encompasses all chemotherapy agent that incorporate platinum or a platinum salt to treat or prevent cancer. In certain aspects of these methods, the platinum-based chemotherapy is an adjuvant therapy. Therefore, the methods described herein predict a patient's response to the use of a platinum-based chemotherapy as either a monotherapy or a combination therapy with other known anti-cancer agents or techniques (e.g. radiation and surgery, for example).

### Response to Treatment for Colorectal Cancer

The disclosure provides methods of prognosing subjects with colorectal cancer (CRC) or metastatic CRC (mCRC) and, furthermore, methods of optimizing treatment by predicting the subject's response to monoclonal antibody therapy, alone, or in combination with cytotoxic chemotherapy. The methods and data described herein identify a specific genomic mutation in a let-7 miRNA binding site within the 3′ untranslated region (UTR) of the KRAS gene, referred to as the KRAS variant.

The disclosure provides a method of prognosing a test subject with early stage colorectal cancer (CRC), including detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of mutation indicates an increased survival rate when compared to a control subject or a subject with advanced CRC (including, for example stage III, stage IV, and metastatic CRC).

The disclosure provides a method of prognosing a patient with advanced colorectal cancer (CRC), including detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of the KRAS-variant indicates a decreased survival rate when compared to a control subject or a subject with early stage CRC. Advanced CRC includes, for example, stage III, stage IV, and metastatic CRC.

The disclosure provides a method of predicting the response of a cancer cell to a monoclonal antibody monotherapy, including detecting a mutation in let-7 complementary site LCS6 of human KRAS in a patient sample, wherein the mutation is a SNP comprising a uracil (U) or thymine (T) to guanine (G) transition at position 4 of LCS6, wherein the presence of the mutation indicates a sensitivity to monoclonal antibody monotherapy. In certain embodiments of this method, the cancer cell is a colorectal cancer (CRC) cell. The cancer cell may be evaluated in vitro or ex vivo. A non-limiting example of the monoclonal antibody monotherapy is Cetuximab.

The disclosure provides a method of predicting the response of a cancer cell to the combination of a chemotherapy and monoclonal antibody therapy, including detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of the mutation indicates a resistance to the combination. In certain embodiments of this method, the cancer cell is a colorectal cancer (CRC) cell. The cancer cell may be evaluated in vitro or ex vivo. A non-limiting example of the monoclonal antibody monotherapy is Cetuximab. The chemotherapy may be a cytotoxic agent. A non-limiting example of the cytotoxic agent is irinotecan. In certain embodiments, treatment of a subject carrying the KRAS-variant with a chemotherapeutic agent (e.g. irinotecan) results in increased expression of the KRAS-variant. When reporter expression is compared following irinitecan exposure in KRAS-variant versus non-variant cancer cells, no change was found in expression of the wild-type 3′UTR reporter. However, a statistically-significant increase in expression in the KRAS-variant 3′UTR reporter was discovered (FIGS. 24A and 24B). The data indicates that irinotecan exposure changes the cellular context in a manner that activates the KRAS-variant allele.

Although the method can be applied to subjects of all ages, in certain embodiments of this method, the test subject a newborn, child, adult, or senior (aged 65 or above). The subject may be pre- or post-menopausal (aged 52 years or older).

Controls or control subjects include healthy individuals and those individuals who have CRC, but who do not carry the KRAS-variant. Moreover, the control subject can be a national average based upon the expected survival of individuals born in the same year as the test subject, or who belong to the same generation as the test subject. In a preferred embodiment, this control value does not include those individuals who carry the KRAS-variant. In certain aspects of this method, the survival rate is an overall survival rate (for instance, some examples, include, but are not limited to, survival rates calculated from the time of cancer development or diagnosis until the subject succumbs to the cancer (death), enters remission, or a doctor declares the subject cured or clean of all cancer cells), five-year survival rate or one-year survival. Shorter survival periods are calculated, for instance, from either the development or diagnosis of the cancer until a determined time, such as one or five years.

The disclosure also provides a method of predicting the response of a colorectal cancer (CRC) cell to a monoclonal antibody based therapy, including detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of the mutation indicates an increased sensitivity to monoclonal antibody based therapy. The CRC cell may be evaluated in vitro or ex vivo. The monoclonal antibody based therapy may be Cetuximab.

The disclosure also provides a method of predicting the response of a colorectal cancer (CRC) cell to a cytotoxic chemotherapy, including detecting a mutation in let-7 complementary site LCS6 of human KRAS in a patient sample, wherein the mutation is a SNP comprising a uracil (U) or thymine (T) to guanine (G) transition at position 4 of LCS6, wherein the presence of the mutation indicates a resistance to cytotoxic chemotherapy. In certain embodiments of this method, the CRC cell is evaluated in vitro or ex vivo. The cytotoxic chemotherapy may be irinotecan. In an embodiment of this method, the cytotoxic chemotherapy is a combinatorial therapy that includes a monoclonal antibody based therapy. The monoclonal antibody based therapy may be Cetuximab.

When the CRC cell is evaluated ex vivo, the cell is obtained from a subject. The subject may be of any age. In certain embodiments of this method the subject is at least 30, 35, 40, 45, 50, 55, 60, 65, 70, 75 years of age, or any age in between.

When the CRC cell is evaluated in vitro, the cell may be isolated, reproduced, or derived from an established cell lines, including a colon or colorectal cancer cell line included in the NCI-60 panel. A CRC cell may be isolated, reproduced, or derived from any colon or colorectal cancer cell line, including, but not limited to, those cell lines that carry the KRAS-variant, either alone, or in combination with a second or additional mutation in KRAS or another gene.

For this method, the preferred monoclonal antibody monotherapy is Cetuximab, however, the monoclonal antibody monotherapy encompasses any monoclonal antibody used to treat or prevent cancer. Preferably, the monoclonal antibody is in part or entirely human or humanized. For this method, the preferred chemotherapy is a cytotoxic chemotherapy such as irinotecan, however, the chemotherapy encompasses any chemotherapy agent that is used to treat or prevent cancer. In certain aspects of this method, the chemotherapy or cytotoxic chemotherapy is an adjuvant therapy. Therefore, this method predicts a patient's response to the use of a monoclonal antibody as either a monotherapy or a combination therapy with a chemotherapy agent or other known techniques for treating or preventing cancer (e.g. radiation and surgery).

### BRIEF DESCRIPTION OF THE DRAWINGS

FIG. 1A-B is a pair of graphs depicting the distribution of the KRAS variant in breast-cancer subtypes in all women (A) and premenopausal (≦51 years) women (B) from study group 2. Data are numbers of cases diagnosed with breast-cancer subtype/numbers of patients tested for the KRAS variant. *p=0.044 versus all other subtypes. †p=0.033 versus all other subtypes.

FIG. 2A-B is a pair of box plot depicting BRCA1 gene expression among the KRAS-variant positive and KRAS-variant negative cases of triple-negative breast cancer. Y-axes are in arbitrary units. (A) BRCA1 probe 1, p=0.06. (B) BRCA1 probe 2, p=0.01.

FIG. 3 is a series of box plots depicting the expression of let-7 family of microRNAs in the KRAS-variant positive versus KRAS-variant negative cases of triple-negative breast cancer. Y-axes are in arbitrary units.

FIG. 4 is a heat map showing the KRAS-variant differentially expressed genes in triple negative breast cancer patients analyzed by LIMMA model. The 50 most significant genes were used for the clustering; p<0.0001 for clustering. KRAS-variant samples are dark gray; wild-type samples are light gray. White have unknown KRAS-variant status.

FIG. 5 is a graph depicting the KRAS-variant in ER/PR+ versus ER/PR− premenopausal breast cancer patients.

FIG. 6 is a series of box graphs depicting Gene expression signatures associated with the KRAS-variant in triple negative breast cancer patient tumors.

FIG. 7 is a graph showing that the KRAS variant predicts significantly worse overall survival for postmenopausal ovarian cancer patients over 52 years of age. Overall survivals for ovarian cancer patients with (n=59) and without (n=220) the KRAS variant are compared using the Kaplan-Meier analysis. Outcome is significantly worse for KRAS variant positive EOC patients over 52 years of age by log-rank test (P=0.0399).

FIG. 8 is a graph showing that the KRAS variant is associated with suboptimal debulking after neoadjuvant chemotherapy. Surgical debulking after neoadjuvant chemotherapy is compared in ovarian cancer patients (n=116) with the KRAS variant (n=26) or without (n=90). By χ² analysis, KRAS-variant patients are significantly more likely to be suboptimally debulked with greater residual disease (RD) than are non-variant patients (P=0.044).

FIG. 9A is a signature of 50 differentially expression gene candidates in KRAS variant (KV) triple-negative breast tumors (TNBC KRAS Signature) that shows higher scores in KV EOC samples than in non-variant samples.

FIG. 9B is a signature of genes associated with KRAS-addicted tumors (KRAS Addiction Signature), which are upregulated in KV EOC tumors.

FIG. 9C a signature of differential expression of the top 20 genes in KV EOC tumors, reflecting a re-analysis of differential gene expression in carboplatin-sensitive and carboplatin-resistant EOC cells.

FIG. 9D is a heat map of the top differentially expressed genes between KV (dark gray) and non-variant (light gray) tumor samples. The color key depicts a spectrum from blue (values 0 to 5) to white (approximately 5), and from white to red (5 to 10). For a color version of this heat map, see Ratner E S, et al. Oncogene, (5 Dec. 2011), 1-8; the contents of which are incorporated herein by reference).

FIG. 10 is a graph showing that the KRAS variant is associated with resistance to carboplatin and carboplatin/taxol chemotherapy in cell lines. Cell lines with the KRAS variant (BG1) and without the KRAS variant (CAOV3) were treated with chemotherapy and half-maximal inhibitory concentration (IC50) is shown on the Y axis, and chemotherapeutic agent on the X axis. Higher IC50 represents resistance to the tested chemotherapeutic agent. BG1=KRAS variant/BRCA wild-type cell line; CAOV3=non-variant/BRCA wild-type cell line; IGR-OV1=KRAS-variant/BRCA1 mutant cell line. Error bars are RSE.

FIG. 11A is a graph showing decreased cell survival in the KRAS-variant line, BG1 (*P<0.001), with no effect on the non-variant line, CAOV3. Cell lines, with (BG1) and without (CAOV3) the KRAS variant, were treated with siRNA/miRNA combinations that bind selectively to the variant allele.

FIG. 11B is a graph showing decreased KRAS protein expression in BG1 (right) concordant with the decrease in cell survival, with no effect on CAOV3 (left). Cell lines, with (BG1) and without (CAOV3) the KRAS variant, were treated with siRNA/miRNA combinations that bind selectively to the variant allele. Different siRNAs are denoted by numbers.

FIG. 12 is a graph depicting Cell lines with the KRAS variant (BG-1 and IGROV1) have significantly lower levels of let-7b compared to a non-variant cell line (CaOV3). Statistical analysis was done with a one way Anovea and Tukey's Multiple comparison test.

FIG. 13A-B is a schematic depicting an alignment of the KRAS-variant sequence with non-variant sequences. Panel A depicts a non-variant sequence of KRAS. Panel B depicts exemplary variant siRNA oligos targeted to the KRAS-variant sequence. In both panels, the underlined sequence depicts the let-7 binding site. In both panels, the boxed nucleotide represents either the wild type (non-variant) nucleotide (A) or the KRAS variant single nucleotide polymorphism (B). siRNAs are shown starting with their 3′ end.

FIG. 14 is a Kaplan-Meier curve for the KRAS variant and cause-specific survival in all cancer stages.

FIG. 15A is a Kaplan-Meier curve for the KRAS variant and cause-specific survival in early-stage (stage I and II) CRC.

FIG. 15B is a Kaplan-Meier curve for the KRAS variant and cause-specific survival in stage III CRC.

FIG. 15C is a Kaplan-Meier curve for the KRAS variant and cause-specific survival in stage IV CRC.

FIG. 16A is a Kaplan-Meier curve for the KRAS variant, KRAS mutations and cause-specific survival in early-stage (stage I and II) CRC, P=0.875.

FIG. 16B is a Kaplan-Meier curve for the KRAS variant, KRAS mutations and cause-specific survival in stage III CRC.

FIG. 16C is a Kaplan-Meier curve for the KRAS variant, KRAS mutations and cause-specific survival in stage IV CRC.

FIG. 17 is a Kaplan-Meier curve for the KRAS variant, MSI status and cause-specific survival in early-stage (stage I and II) CRC.

FIG. 18A is a graph depicting the median progression free survival according to the KRAS LCS6 genotype status in patients treated with anti-EGFR moAbs monotherapy or in combination with chemotherapy as salvage treatment.

FIG. 18B is a graph depicting the median overall survival according to the KRAS LCS6 genotype status in patients treated with anti-EGFR moAbs monotherapy or in combination with chemotherapy as salvage treatment.

FIG. 19A is a graph depicting the median progression-free survival according to the KRAS LCS6 genotype status in all patients treated with anti-EGFR moAbs monotherapy as salvage treatment.

FIG. 19B is a graph depicting the median progression-free survival according to the KRAS LCS6 genotype status in all patients treated with anti-EGFR moAbs based combination chemotherapy as salvage treatment.

FIG. 19C is a graph depicting the median progression-free survival according to type of therapy in all KRAS variant carriers.

FIG. 19D is a graph depicting the median progression-free survival according to type of therapy in all non-KRAS variant carriers.

FIG. 20A is a graph depicting the median progression-free survival according to the KRAS LCS6 genotype status in the double (KRAS and BRAF) wt patients' population treated with anti-EGFR moAbs monotherapy as salvage treatment.

FIG. 20B is a graph depicting the median progression-free survival according to the KRAS LCS6 genotype status in the double (KRAS and BRAF) wt patients' population treated with anti-EGFR moAbs based combination chemotherapy as salvage treatment.

FIG. 20C is a graph depicting the Median progression-free survival according to type of therapy in the double (KRAS and BRAF) wt KRAS variant carriers

FIG. 20D is a graph depicting the Median progression-free survival according to type of therapy in the double (KRAS and BRAF) wt non-KRAS variant carriers.

FIG. 21A is a graph depicting the median overall survival according to the KRAS LCS6 genotype status in all patients treated with anti-EGFR moAbs monotherapy as salvage treatment.

FIG. 21B is a graph depicting the median overall survival according to the KRAS LCS6 genotype status in all patients treated with anti-EGFR moAbs based combination chemotherapy as salvage treatment.

FIG. 21C is a graph depicting the median overall survival according to type of therapy in all KRAS variant carriers.

FIG. 21D is a graph depicting the median overall survival according to type of therapy in all non-KRAS variant carriers.

FIG. 22A is a graph depicting the median overall survival according to the KRAS LCS6 genotype status in the double (KRAS and BRAF) wt patients' population treated with anti-EGFR moAbs monotherapy as salvage treatment.

FIG. 22B is a graph depicting the median overall survival according to the KRAS LCS6 genotype status in the double (KRAS and BRAF) wt patients' population treated with anti-EGFR moAbs based combination chemotherapy as salvage treatment.

FIG. 22C is a graph depicting the median overall survival according to type of therapy in the double (KRAS and BRAF) wt KRAS variant carriers.

FIG. 22D is a graph depicting the median overall survival according to type of therapy in the double (KRAS and BRAF) wt non-KRAS variant carriers.

FIG. 23A is a graph depicting the median progression-free survival according to type of therapy in the KRAS and BRAF mutated KRAS variant carriers.

FIG. 23B is a graph depicting the median progression-free survival according to type of therapy in the KRAS and BRAF mutated non-KRAS variant carriers.

FIG. 23C is a graph depicting the median overall survival according to type of therapy in the KRAS and BRAF mutated KRAS variant carriers.

FIG. 23D is a graph depicting the median overall survival according to type of therapy in the KRAS and BRAF mutated non-KRAS variant carriers.

FIG. 24A is a graph depicting the normalized luciferase expression in wild type KRAS and KRAS-variant cancer cells following treatment with the chemotherapeutic agent irinotecan.

FIG. 24B is a graph depicting the fold repression (expressed as KRAS variant/KRAS wild type) as a function of irinotecan concentration, when cancer cells are treated with irinotecan.

### DETAILED DESCRIPTION

A functional variant in a let-7 microRNA complementary site in the 3′UTR of the KRAS oncogene (rs61764370) associated with cancer was previously identified (International Patent Application No. PCT/US2008/065302, the contents of which are incorporated herein by reference in their entirety). An investigation of the association of this variant with cancer tumor biology is described herein.

### Breast Cancer

Breast tumors are classified into ER (estrogen) and/or PR (progesterone) receptor positive, HER2 (Her2/neu/ERBB2) amplified, and triple-negative tumors (i.e., ER/PR negative and HER2 negative) (Sørlie T, et al. Proc Natl Acad Sci USA 2001; 98: 10869-74). Gene expression and receptor profiling further classifies breast cancer into four biological subgroups: The luminal A (ER- and/or PR-receptor positive, HER2-negative) tumors, luminal B (ER- and/or PR-receptor positive, HER2 positive), HER2-positive (HER2-positive, ER/PR negative) and basal like (ER/PR/HER2-negative, also referred to as triple negative breast cancer (TNBC)) tumors (Sørlie T, et al. Proc Natl Acad Sci USA 2001; 98: 10869-74).

Triple negative breast cancer (TNBC) is the most aggressive subclass with worse cause-specific survival at 5 years compared to the other subtypes (Haffty B G et al. J Clin Oncol 2006; 24: 5652-57). Recent transcriptional profiling studies suggest there is further heterogeneity within TNBC and these tumors can be categorized into two broad subgroups; the ER/PR/HER2 (triple) negative tumors that express EGFR or cytokeratin (CK) 5/6, and, therefore, termed ‘basal-like’, and the ER/PR/HER2 (triple) negative tumors that do not express EGFR or CK5/6. The basal-like triple negative (TN) tumors are also characterized by an earlier age (or younger age) of onset than non-basal-like forms and low expression of BRCA1 (BReast CAncer 1); the basal-like phenotype is common among carriers of the BRCA1 mutation (Rakha EA and Ellis IO. Pathology 2009; 41: 40-47). An aberrant luminal progenitor cell population (that may be ER positive) is a target for transformation in BRCA-1-associated basal tumors (Lim E, et al. Nat Med 2009; 15: 907-13). Although prognostic gene-expression markers are highly divergent, several modules such as DNA repair deficiency, signatures of immune response, or transition from epithelium to mesenchyme are commonly noted in a subset of these tumors (Bild A H, et al. Breast Cancer Res 2009; 11: R55). Identification of the drivers of these transcriptional modules is one approach for discovery of specific and personalized therapies.

Association of the triple-negative breast cancer phenotype with young age of onset and an absence of association with known risks or reproductive factors (Yang X R, et al. Cancer Epidemiol Biomarkers Prey 2007; 16: 439-43) suggests there are genetic risks for development of this cancer (Bauer K R et al. Cancer 2007; 109: 1721-28). Prior to this disclosure, few genetic markers of such increased risk existed. Although BRCA1 mutations are often associated with triple-negative tumors, these mutations are rare and account for only 10-15% of patients with triple-negative breast cancer, dependent on ethnic background and family history (Young S R, et al. BMC Cancer 2009; 9: 86; Nanda R, et al. JAMA 2005; 294: 1925-33).

The studies provided herein determined the frequency distributions of the KRAS variant in 415 patients with histologically confirmed breast cancer and 457 controls from Connecticut, USA (study group 1) as well as an association of this variant with breast cancer subtypes in 690 Irish women with known estrogen receptor (ER), progesterone receptor (PR), and HER2 statuses, and 360 controls (study group 2). Data for study groups 1 and 2 was pooled with a cohort of 140 women with triple-negative breast cancer and 113 controls to assess the association of the KRAS variant with triple-negative breast cancer risk as well as genome-wide mRNA and specific miRNA expression in patients with triple-negative breast cancer.

Although frequency distributions of the KRAS variant in study group 1 did not differ between all genotyped individuals, eight (33%) of 24 premenopausal women with ER/PR-negative cancer had the KRAS variant, compared with 27 (13%) of 201 premenopausal controls (p=0.015). In study group 2, the KRAS variant was significantly enriched in women with triple-negative breast cancer (19 [21%] of 90 cases) compared with 64 (13%) of 478 for luminal A, 13 (15%) of 87 for luminal B, and two (6%) of 35 for HER2-positive subgroups (p=0.044). Multivariate analysis in the pooled study groups showed that the KRAS variant was associated with triple-negative breast cancer in premenopausal women (odds ratio 2.307, 95% CI 1.261-4.219, p=0.0067). Gene-expression analysis of triple negative breast-cancer tumors suggested that KRAS-variant positive tumors have significantly altered gene expression, and are enriched for the luminal progenitor and BRCA1 deficiency signatures. miRNA analysis suggested reduced levels of let-7 miRNA species in KRAS-variant tumors.

The KRAS variant is a genetic marker for development of triple-negative breast cancer in premenopausal women. Altered gene and miRNA expression signatures enable molecular and biological stratification of patients with triple negative breast cancer.

### Colorectal Cancer

The KRAS variant is a prognostic biomarker in early-stage colorectal cancer (CRC). Moreover, the KRAS variant induces higher levels of the KRAS oncogenic protein and lower levels of the tumor suppressor lethal-7 (let-7) miRNAs. The influence of KRAS-variant was studied in 409 early-stage (stage I and II), 182 stage III and 69 stage IV cases from the large, prospective Netherlands Cohort Study (NLCS). Early-stage patients with the KRAS variant had a better prognosis, especially those that also had additional KRAS mutations. This discovery is independent of microsatellite-instability or other prognostic factors. In addition, the influence of the KRAS-variant on CRC risk was also studied by using data from 1,886 subcohort members from the NLCS. The G-allele (i.e., the KRAS variant allele) was not associated with a likelihood of developing CRC overall, but was enriched in advanced stage CRC, suggesting it may predict presenting with more advanced disease. Because this study population is the only untreated population analyzed to date, these results provide a novel insight into the natural biology of colorectal cancer with the KRAS variant.

As the data presented herein demonstrate, the KRAS variant is a new biomarker in colorectal cancer (CRC) to guide treatment decisions in early-stage patients. Early-stage CRC cases with the KRAS variant have a better outcome, however, in advanced disease, this better outcome no longer exists. For early-stage patients, the combination of the KRAS variant genotype and at least one KRAS mutation is also a prognostic biomarker of better outcome to be considered in therapy-decision-making.

Despite diagnostic and therapeutic innovations, colorectal cancer (CRC) remains the second leading cause of cancer death in the western world. The tumor-node-metastasis-system (TNM) is currently the standard tool to provide prognostic information. The TNM system is highly predictive for prognosis at the extremes (early and late stage CRC), but less predictive for intermediate stages. According to current guidelines, adjuvant chemotherapy is not given to early-stage patients (i.e., T1-3-N0-M0, according to the International Union Against Cancer TNM). Five-year survival rates in this group of early-stage patients (i.e., T1-3-N0-M0) are greater than 70%. Nevertheless, 20-30% of early-stage patients (stage I and II) will die of CRC within 5 years, evoking the question whether these deaths could have been avoided if these patients were identified in advance and therapy was adapted accordingly. Previously, numerous studies have been published claiming a prognostic influence of molecular markers. In contrast to the assertions of these previous reports, the results of these studies are inconsistent. Thus, prior to the development of the methods described herein, the question of which molecular alterations influence prognosis remained unresolved (Smits K M, et al. Pharmacogenomics. 2008; 9(12): 1903-16).

MicroRNAs (miRNA), have been identified as important factors in cancer development and progression. Evidence suggests that a single miRNA can regulate many mRNAs simultaneously (Paranjape T, et al. Gut. 2009; 58(11): 1546-54). Moreover, miRNAs can act as both tumor suppressors and oncogenes (Johnson S M, et al. Cell. 2005; 120(5): 635-47). The lethal-7 (let-7) family of miRNAs is one of the first miRNA families to be discovered. The expression of let-7 family miRNAs is altered in many cancers. For example, in lung cancer, let-7 is poorly expressed (Calin G A, et al. Proc Natl Acad Sci USA. 2004; 101(9): 2999-3004; Takamizawa J, et al. Cancer Res. 2004; 64(11): 3753-6), overexpression of let-7 inhibits cell growth in vitro (Takamizawa J, et al. Cancer Res. 2004; 64(11): 3753-6) and in vivo (Kumar M S, et al. Proc Natl Acad Sci USA. 2008; 105(10): 3903-8; Esquela-Kerscher A, et al. Cell Cycle. 2008; 7(6): 759-64) suggesting that let-7 miRNAs may act as tumor suppressors (Johnson S M, et al. Cell. 2005; 120(5): 635-47).

In colon cancer cells, let-7 expression is significantly decreased in tumor tissue as compared to adjacent non-cancerous tissue (Akao Y, et al. Biol Pharm Bull. 2006; 29(5): 903-6). Moreover, let-7 expression was increased and RAS expression was decreased in cell lines after transfection of a let-7a-1 miRNA precursor, suggesting that let-7 is involved in regulating colon cancer cell growth (Akao Y, et al. Biol Pharm Bull. 2006; 29(5): 903-6).

MiRNAs can control gene expression by binding to complementary elements in the 3′ untranslated region (UTR) of target mRNAs. Let-7 induces RAS downregulation after binding to specific sites in the 3′-UTR of KRAS mRNA. The KRAS variant affects let-7 mediated regulation of KRAS expression. The occurrence of the variant G-allele (i.e., the KRAS variant) leads to higher KRAS levels and lower let-7 levels as compared to the wild type. G-allele carriers have an increased lung cancer risk in moderate smokers, an increased ovarian cancer risk (particularly for post-menopausal women), an increased risk of developing breast cancer (and, in particular, the triple negative breast cancer subtype), and a reduced survival in oral cancers but not in lung cancer. In KRAS/BRAF mutated CRC, G-allele carriers (KRAS variant carriers) showed a reduced survival in late-stage CRC and an altered response to cetuximab, demonstrating a role of the KRAS variant in colon cancer. Because the role of the KRAS variant genotype in early-stage CRC was unresolved, the experiments and data presented herein assessed the influence on prognosis in 409 early-stage (TNM stage I and II; T1-4, N0, M0), 182 stage III (T1-4, N1, M0) and 69 stage IV (T1-4, NO-1, MD CRC cases from a large prospective cohort study. The influence of KRAS variant genotype on CRC risk was also assessed by using data from 1,886 subcohort members from the NLCS.

The results of this study demonstrate that a T>G variant in the LCS6 in the 3′ UTR region of KRAS affects prognosis in early-stage (stage I and II) CRC. The KRAS variant was present in 16.4% of the cases, whereas it is found in only 6% of world populations (Chin L J, et al. Cancer Res 2008; 68:8535-40), and 12% to 15% in persons from European descent (Ratner E, et al. Cancer Res 2010; 70:6509-15). An increased frequency of the KRAS variant (G-allele) was discovered in advanced cases (early stage 14%, 19.2%, and 21.4% in stage III and IV patients, respectively), which is comparable with previously reported frequencies in stage III (Graziano F, et al. Pharmacogenomics J 2010; 10:458-64). The G-allele (KRAS variant) was found in 18% of the subcohort members. A statistically significant association was discovered between the KRAS-variant and an increased presentation with advanced colon cancer, providing valuable insight into the natural biology of colon cancer in KRAS variant carriers. Furthermore, a statistically significant increase in survival for early-stage CRC cases with the KRAS variant was discovered; among KRAS-mutated patients none of the early-stage patients carrying the G-allele (KRAS variant) died from CRC. This statistically significant increase in survival for early-stage CRC cases with the KRAS variant was independent of other prognostic factors such as tumor differentiation or sublocation. Because T4 tumors were rare in the study group of early-stage cases, a higher frequency of stage IIb cases among KRAS wild types is ruled out as the cause of the observed worse outcome. A statistically significant effect was not found in stage III or IV, although the results indicate a worse prognosis for stage III cases with the KRAS variant (G allele) and KRAS mutations. In addition, the effect of the KRAS variant (G allele) on CRC risk was studied. A decreased risk of early-stage CRC was found, but no effect on the risk of advanced stage CRC, indicating that the G-allele (KRAS variant) is not associated with a higher likelihood of developing CRC overall.

In previous studies, mutations in KRAS have been associated with a poorer prognosis. However, results on this topic are inconsistent and, furthermore, the clinical relevance of these results are unclear (Smits K M, et al. Pharmacogenomics 2008; 9:1903-16). Acquired KRAS mutations are not the same as the KRAS variant, which is a congenital mutation, and, therefore, has a different effect on tumor development, biology, and thus prognosis.

The discovery that the KRAS variant is associated with an increased survival in early-stage CRC is intriguing. Previous research has suggested that cellular senescence can be triggered by overexpression of oncogenic Ras and might contribute to growth cessation in premalignant or benign neoplasms (Mooi WJ and Peeper DS. N Engl J Med 2006; 355:1037-46). Tumor cell senescence has been reported in human cancers. Premalignant colon adenomas display features of senescence as well (Collado M and Serrano M. Nat Rev Cancer 2010; 10:51-7). Oncogene-induced senescence may play a role in premalignant lesions only. Nevertheless, physiologic levels of KRAS can induce senescence in the absence of the transcription factor Wilms tumor 1 (WT1) (Vicent S, et al. J Clin Invest 2010; 120:3940-52). Lung cancer patients with high KRAS gene expression had a good prognosis if they had decreased expression of WT1 related genes (Vicent S, et al. J Clin Invest 2010; 120:3940-52). Together, these results imply that other molecular factors can be involved in the determination of cell fate, and that oncogene-induced senescence can occur after an altered expression of other genetic or epigenetic targets. Oncogene-induced senescence could also play a role in CRC: the KRAS-LCS6 genotype could either lead to an advanced stage tumor, or an early-stage tumor with a better prognosis based on the other (epi)genetic markers that are affected.

A better outcome was found for early-stage (stage I and II) cases with the KRAS variant and BRAF mutations or RASSF1A hypermethylation, both of which are involved in the Ras signaling pathway. BRAF-associated senescence has previously been reported to occur in melanoma (Michaloglou C, et al. Nature 2005; 436:720-4) but a possible role of RASSF1A in oncogene-induced senescence has not been demonstrated. As in the study population described herein, the coincidence of the KRAS variant with either a BRAF mutation and/or RASSF1A hypermethylation is less common, and, therefore, statistical significance was not reached. When combining these (epi)genetic events, the better outcome of patients with a combination of the KRAS variant (G-allele) and an alternation of KRAS, BRAF, or RASSF1A was even more enhanced. Thus, Ras overexpression due to the KRAS variant (G-allele), in combination with (epi)genetic alterations in genes from the Ras pathway, could induce senescence in early-stage CRC, thereby influencing survival. For advanced-staged cases, an increasing number of molecular pathways are affected that influence prognosis.

The let-7 family of miRNA demonstrate a tumor growth suppression effect with decreased let-7 expression and increased KRAS levels in the presence of the KRAS variant compared to wild type (13). Accordingly, patients with the KRAS-variant are expected to have a worse prognosis, as shown for, for instance, in oral cancer (Christensen B C, et al. Carcinogenesis 2009; 30:1003-7). For CRC, there are two reports studying the effect of KRAS genotype on outcome in treated patients (Graziano F, et al. Pharmacogenomics J 2010; 10:458-64; Zhang W et al. Ann Oncol 2011; 22:104-9). The first reports poor survival among a small population of irinotecan-refractory metastatic patients with the KRAS-variant treated with Irinotecan and Cetuximab, as well as an association with KRAS mutations and the absence of BRAF mutations (Graziano F, et al. Pharmacogenomics J 2010; 10:458-64), however, these findings could not be replicated in this study as patients were primarily untreated. The second reports a better response to cetuximab alone in metastatic CRC and a longer survival in patients with the KRAS variant without KRAS mutations, but the response was not statistically significant (Zhang W et al. Ann Oncol 2011; 22:104-9). The data presented herein demonstrate a better prognosis in stage IV KRAS variant carriers, although the comparison is not statistically significant, which may be explained by the small size of the group of stage IV patients. Other studies used germline tissue to assess the KRAS genotype, however, the studies described herein used tumor DNA to assess KRAS genotype. It is well documented that genotype of normal and tumor tissue is the same for the KRAS variant.

The seemingly discordant results in early and advanced stage CRC raises questions on the origin and progression of tumors in different cancer stages, and whether early-stage CRC might develop through a molecular distinct pathway as compared with advanced stage. The KRAS-variant is more common among cases with advanced stage disease, however, patients who are diagnosed early with the KRAS variant seem to have a more advantageous outcome. Thus, the data imply a different biology in early-stage as compared with advanced stage cases. The discovery that early-stage KRAS wild-type patients have a poor prognosis, even if they have a MSI tumor, might indicate that these patients would benefit from additional adjuvant treatment. Further research, including randomized clinical trials, is needed to assess whether these early-stage patients with a poor prognosis would benefit from additional adjuvant treatment. Prior to the discovery of the biomarkers and methods described herein, MSI has been considered to be a marker for good prognosis (Boland CR and Goel A. Gastroenterology 2010; 138:2073-87.e3) however, the data from this study demonstrate a better outcome for KRAS variant allele carriers independent of MSI status.

The analysis presented herein of the influence of the KRAS variant in early-stage CRC cases demonstrates a better outcome for early-stage G-allele (KRAS variant) carriers with KRAS mutations. The population used in this study is the only group studied to date that is generally untreated, and for the first time, the data gathered from this study provides a valuable insight into the natural biology of early stage CRC with the KRAS variant. Consequently, the evidence presented herein is the first indication that the KRAS variant genotype is a possible prognostic biomarker for early-stage CRC that can be used to identify CRC patients with a good prognosis.

### Response to Treatment

### Ovarian Cancer

Epithelial ovarian cancer (EOC) is the second most common female pelvic reproductive organ cancer in the United States, and carries the highest mortality in this category in the Western world. It is the fifth overall leading cause of cancer death in females in the United States, with 13,850 women dying from this disease yearly. Despite multiple new approaches to treatment, the high rates of death from EOC have remained largely unchanged for many years, with a 5-year overall survival of only 30-39% (Parmar M K, et al. (2003). Lancet 361: 2099-2106).

The standard chemotherapy regimen to treat EOC currently used is carboplatin and paclitaxel (Pfisterer J, et al. (2006). J Clin Oncol 24: 4699-4707), based on prospective randomized trials (Herzog T and Pothuri B. (2006). Nat Clin Pract Oncol 3: 604-611; Esquela-Kerscher A and Slack F. (2006). Nat Rev Cancer 6: 259-269; Iorio M, et al. (2007). Cancer Res 67: 8699-8707). Although some patients are initially resistant to platinum-based chemotherapy (referred to as ‘platinum resistant’), developing recurrence within 6 months of treatment, it is the first line treatment given to all EOC patients. An improved understanding of the fundamental biological differences in EOC tumors that could explain platinum resistance among EOC patients would allow a more rational selection of treatments (Parmar M K, et al. (2003). Lancet 361: 2099-2106; Pfisterer J, et al. (2006). J Clin Oncol 24: 4699-4707; Herzog T and Pothuri B. (2006). Nat Clin Pract Oncol 3: 604-611).

MicroRNAs (miRNAs) are a class of 22-nucleotide noncoding RNAs that are aberrantly expressed in virtually all cancer types, where they can function as a novel class of oncogenes or tumor suppressors. In EOC, in addition to distinguishing normal ovarian tissue from malignant ovarian tissue (Iorio M, et al. (2007). Cancer Res 67: 8699-8707; Zhang L, et al. (2008). Proc Natl Acad Sci USA 105: 7004-7009), miRNA expression patterns have been shown to be important in EOC pathogenesis (Mezzanzanica D, et al. (2010). Int J Biochem Cell Biol 42: 1262-1272; van Jaarsveld M, et al. (2010). Int J Biochem Cell Biol 42: 1282-1290) and are associated with altered EOC patient outcome (Eitan R, et al. (2009). Gynecol Oncol 114: 253-259) and response to treatment (Lu L, et al. (2011). Gynecol Oncol 122: 366-371). miRNA expression differences have also been associated with chemotherapy and platinum resistance in EOC (Eitan R, et al. (2009). Gynecol Oncol 114: 253-259; Lu L, et al. (2011). Gynecol Oncol 122: 366-371; Chen K, et al. (2008). Carcinogenesis 29:1306-1311).

Additional insight into the importance of miRNAs in cancer has come from the discovery of inherited single-nucleotide polymorphisms that disrupt miRNA coding sequences (Chin L J, et al. (2008). Cancer Res 68: 8535-8540) and miRNA-binding sites in the 3′ untranslated regions (3′ UTRs) of oncogenes (Chen K, et al. (2008). Carcinogenesis 29:1306-1311; Chin L J, et al. (2008). Cancer Res 68: 8535-8540). An example of such a functional variant is rs61764370, referred to as the KRAS variant, which is located in the KRAS 3′ UTR in a let-7 miRNA complementary site. An association between rs61764370 and epithelial ovarian cancer (EOC) risk was previously reported (see, International Patent Application No. PCT/US2008/065302 and International Patent Application No. PCT/US2010/023412; the contents of which are each herein incorporated in their entireties). Furthermore, the methods and examples provided demonstrate that this variant is a biomarker of clinical outcome and chemotherapy resistance in epithelial ovarian cancer (EOC). The evidence supports a continued functional role of the KRAS variant in tumors, an association with aggressive tumor biology and poor cancer-specific outcome.

The potential of the KRAS variant to act as a biomarker of outcome in EOC in both the presence and the absence of deleterious BRCA mutations is evaluated herein. Moreover, the potential cause of altered outcome in KRAS-variant EOC patients is determined by studying the response to neoadjuvant platinum-based chemotherapy, assessing platinum resistance and evaluating EOC tumor gene expression. The data demonstrate that directly targeting of this gain-of-function KRAS variant could reduce cell growth and survival in EOC cell lines with this lesion.

The KRAS variant is a biomarker of poor outcome for postmenopausal women (over 52 years of age) with EOC. The poor outcome in KRAS variant-associated ovarian cancer is due, at least in part, to the association of the KRAS variant with resistance to platinum-based chemotherapy, based on a worse response to neoadjuvant platinum-based chemotherapy, and statistically significantly increased platinum resistance in adjuvantly-treated EOC patients with the KRAS variant.

The biological differences between KRAS-variant EOC and nonvariant EOC tumors are supported by gene expression data, which indicates that KRAS addiction and AKT-mediated platinum resistance in KRAS-variant-associated EOC. Platinum resistance was further confirmed in vitro in an ovarian cancer cell line with the KRAS variant as compared with a non-variant line. Evidence for the continued dependence of KRAS variant-associated EOC on the KRAS variant germline lesion was shown through direct targeting of this mutation, which led to significant inhibition of both tumor growth and cell survival in a KRAS-variant EOC cell line versus non-variant EOC lines.

The association of the KRAS variant with poor survival for postmenopausal women could be due to underlying biology associated with this variant. In support of the hypothesis that the discovered association reflects underlying biology, the KRAS variant is associated with postmenopausal ovarian cancer (Ratner E, et al. (2010). Cancer Res 15: 6509-6515), with a median age of diagnosis near 59 years of age. Relative survival varies by age, with older women twice as likely to die within 5 years of diagnosis of EOC, further supporting the hypothesis that postmenopausal women may have biologically different tumors than younger women (ACS (2010). Cancer facts & figures 2010. Cancer Facts & Figures. ACS: Atlanta, Ga., pp 1-56). Furthermore, the KRAS variant has been shown to be a biomarker of TNBC risk in premenopausal women, aged <52 years. Thus, the role of the KRAS variant in cancer risk and biology in different tissues may depend on miRNA expression alterations in response to physiologic conditions, such as menopause. Women with the KRAS variant may be first at risk for breast cancer and then, subsequently, be at risk for developing postmenopausal ovarian cancer.

The discovery that the KRAS variant does not predict for poor outcome in a cohort of EOC patients with known deleterious BRCA mutations may be partially explained by the fact that BRCA mutations are associated with platinum sensitivity. Consequences of BRCA mutations associated with platinum sensitivity may occur downstream of any resistance caused or exacerbated by the KRAS variant to platinum agents. It is possible that the younger patients in the study presented herein could have had undocumented deleterious BRCA mutations. Alternatively, or in addition, the younger patients in the study presented herein may also have had other subtypes of ovarian cancer seen more frequently in younger women, such as borderline tumors, resulting in a misdiagnosis of these patients. Although the data provided herein were extensively clinically annotated, BRCA status was not obtained on all of our EOC patients, and although pathology reports were available, tumor tissue was not available for re-review. A recent study that failed to find the association of the KRAS variant with poor outcome and resistance to therapy in EOC used ovarian collections used for genome-wide association studies that had very limited clinical information, i.e., factors such as BRCA status and ovarian cancer specific survival were not available nor included in their analyses (Pharoah P, et al. (2011). Clin Cancer Res 17: 3742-3750).

Similar gene mis-expression patterns were found in two different types of KRAS variant-associated tumors, indicating that these tumors, regardless of tissue of origin, use similar pathways in oncogenesis. Direct targeting of the KRAS-variant lesion in KRAS variant-associated EOC cell lines leads to significantly enhanced cell death and a reduction in KRAS levels. These discoveries suggest a continued critical dependence of KRAS-variant tumors on this single, non-coding germline lesion. Although there has been a significant effort to tailor cancer treatment by measuring tumor gene expression and determining tumor-acquired mutations, there are few, if any, germline variants that have previously been shown to be critical targets for therapy in cancer.

Based upon the data provided herein, it is determined that the KRAS variant is a functional cancer mutation that is important in ovarian cancer and that the KRAS variant allows meaningful subclassification of the ovarian tumors with which it is associated. These discoveries are useful for improving ovarian cancer patient outcome.

### Colorectal Cancer

The incorporation in metastatic colorectal cancer (mCRC) clinical practice of two monoclonal antibodies targeting epidermal growth factor receptor (anti-EGFR moAbs), cetuximab and panitumumab, either used as monotherapy or in combination with chemotherapy, provides a modest clinical benefit in pretreated patients (Cunningham D, et al. N Engl J Med 2004; 351(4):337-345; Saltz L B, et al. J Clin Oncol 2004; 22(7):1201-1208; Saltz L B, et al. N Engl J Med 2000; 343(13):905-914; Van C E, et al. J Clin Oncol 2007; 25(13):1658-1664). Nevertheless, it soon became evident that their efficacy was restricted to a subset of patients. Non-randomized retrospective studies (Amado R G, et al. J Clin Oncol 2008; 26(10):1626-1634; De R W, et al. Ann Oncol 2008; 19(3):508-515; Lievre A, et al. Cancer Res 2006; 66(8):3992-3995; Lievre A, et al. J Clin Oncol 2008; 26(3):374-379; Moroni M, et al. Lancet Oncol 2005; 6(5):279-286; Sartore-Bianchi A, al. J Clin Oncol 2007; 25(22):3238-3245), retrospective analysis of prospective randomized trials (Bokemeyer C, et al. J Clin Oncol 2009; 27(5):663-671; Douillard J et al. AnnOncol supp. 2009; Karapetis C S, et al. N Engl J Med 2008; 359(17):1757-1765; Tol J, et al. N Engl J Med 2009; 360(6):563-572; Van Cutsem E, et al. N Engl J Med 2009; 360(14):1408-1417), and a grand European consortium study (De R W, et al. Lancet Oncol 2010; 11(8):753-762) demonstrated that the presence of tumor acquired KRAS mutations were predictive of resistance to anti-EGFR moAbs therapy and were associated with a worse prognosis and a shorter survival. While for some years now the KRAS mutational status is mandatory for the initiation of anti-EGFR moAb treatment, the issue is unresolved, since, approximately 50-65% of the mCRC patients with KRAS wt tumors derive no benefit from these treatments, implying that additional genetic determinants of resistance or perhaps sensitivity exist (De R W, et al. Ann Oncol 2008; 19(3):508-515; Allegra C J, et al. J Clin Oncol 2009; 27(12):2091-2096; De R W, al. Lancet Oncol 2010; 11(8):753-762; Roock WD, et al. Lancet Oncol 2010). Mounting evidence indicates that the BRAF V600E mutation confers resistant to anti-EGFR MoAbs (De R W, et al. Lancet Oncol 2010; 11(8):753-762; Di N F, et al. J Clin Oncol 2008; 26(35):5705-5712; Laurent-Puig P, et al. J Clin Oncol 2009; 27(35):5924-5930; Saridaki Z, et al. IPLoS One 2011; 6(1):e15980; Souglakos J, et al. Br J Cancer 2009; 101(3):465-472), whereas, although not entirely clear yet, PIK3CA-mutant tumors seem to derive no or little benefit from such a treatment (De R W, et al. Lancet Oncol 2010; 11(8):753-762; Prenen H, et al. Clin Cancer Res 2009; 15(9):3184-3188; Sartore-Bianchi A, et al. Cancer Res 2009; 69(5):1851-1857; Jhawer M, et al. Cancer Res 2008; 68(6):1953-1961; Ogino S, et al. J Clin Oncol 2009; 27(9):1477-1484).

In addition to the tumoral genetic characteristics, there is mounting evidence that the germline genome of patients might also play a role in granting resistance or sensitivity to anti-EGFR moAbs therapy. In support of this notion, polymorphisms in the genes encoding for FcγRIIa and FcγRIIIa, EGFR, EGF, cyclinD 1 and COX-2 have been associated with outcome in mCRC patients treated with cetuximab administered both as monotherapy and in combination with chemotherapy.

MicroRNAs (miRNAs) are an abundant class of highly conserved, endogenous, non-coding, small RNA molecules, 18-25 nucleotides in length, which negatively regulate gene expression by binding to partially complementary sites in the 3′-untranslated region (UTR) of their target mRNAs. Upon processing by Dicer and Drosha RNase III endonucleases, mature miRNAs can suppress mRNA translation by directing an RNA-induced silencing complex to the target mRNA. miRNAs regulate of a number of genes involved in basic biological processes such as proliferation, cellular differentiation and apoptosis, and act as important players in cancer development and progression by behaving either as oncogenes or as tumor suppressors. Although more than 700 miRNA sequences have been recognized in the human genome to date, this number is expected to double. Furthermore, each miRNA can control hundreds of genes by regulating many mRNAs simultaneously.

MiRNA binding to mRNAs is critical for the regulation process of mRNA levels and subsequent protein expression, and this regulation can be affected by single-nucleotide polymorphisms (SNPs) occurring in the miRNA target sites. These SNPs can either create erroneous binding sites or abolish (eliminate) the correct ones, leading to resistance to miRNA regulation and reflecting another kind of genetic variability capable of playing a role in human diseases like cancer (or conferring an increased risk for certain diseases like cancer). Emerging research focuses on the systematic genomic evaluation of these sites and the functional and biological relevance of the detected SNPs, which are significant molecular markers in the rapidly growing area of personalized medicine. Such SNPs appear to affect not only gene expression, but also tumor biology and drug response and drug resistance.

The Lethal-7 (let-7) family of miRNAs was among the first discovered and its differential expression has been detected in a number of cancers. The KRAS oncogene is a direct target of the let-7 miRNA family, and more precisely, let-7 was shown to induce KRAS downregulation upon binding to certain sites in the 3′ untranslated region (3′-UTR) of the KRAS mRNA.

The KRAS variant is a functional single nucleotide polymorphism (SNP) that occurs in a let-7 complementary site (LCS) in the KRAS 3′-UTR mRNA. This SNP (rs61764370) results from a T to G base substitution, which was found to alter the binding capability of mature let-7 to the KRAS mRNA and results in both an increased expression of the KRAS oncogenic protein in vitro and lower let-7 miRNA levels in vivo, possibly due to a negative feedback loop. Consistent with the oncogenic nature of the KRAS gene, the KRAS variant (also referred to as the G-allele) has been shown to confer an increased non-small cell lung cancer (NSCLC) risk in moderate smokers, an increased risk for the development of triple negative breast cancer and, in a subset of women, ovarian cancer. In addition, an increased frequency of the KRAS variant allele was detected among BRCA1 carriers in a small cohort. Furthermore, KRAS variant (G-allele) carriers with head and neck cancer, but not NSCLC, exhibited reduced overall survival. Statistically significantly worse survival and platinum resistance was found in ovarian cancer patients with the KRAS variant (G-allele). Together, the evidence demonstrates a functional and clinical significance of the KRAS variant (also known as the KRAS 3′-UTR LCS6 SNP).

In the mCRC targeted anti-EGFR moAb therapy setting to date, the KRAS variant has been evaluated in two studies with small selected populations and with contradicting and conflicting results (Graziano F, et al. Pharmacogenomics J 2010; 10(5):458-464; Zhang W, et al. Ann Oncol 2011; 22(1):104-109). In the first study (Graziano F, et al. Pharmacogenomics J 2010; 10(5):458-464) within a patient population with KRAS and BRAF wt alleles, and treated with salvage irinotecan-cetuximab combination therapy, KRAS variant (G-allele) carriers were shown to have a statistically significant worse progression free survival (PFS) and overall survival (OS). In contrast, in the second study (Zhang W, et al. Ann Oncol 2011; 22(1):104-109)), where patients were exposed to salvage cetuximab monotherapy, KRAS variant (G-allele) carriers exhibited a longer PFS and OS and had a better objective response rate (ORR). While these studies seem to have opposite results, these patients were not treated identically, and in fact, the addition of irinotecan chemotherapy to cetuximab was also found to predict a poor response in KRAS variant (G-allele) carriers (Winder T, et al. J. Clin. Oncol. [27 (15S Suppl)]. 2009. Abstract). The evidence indicates that unlike tumor acquired KRAS protein mutations, the combination of therapy given to KRAS variant (G-allele) carriers differentially impacts response to cetuximab (Winder T, et al. J. Clin. Oncol. [27 (15S Suppl)]. 2009. Abstract). This is in agreement with data that such miRNA binding site variants are dynamically regulated in disease.

In this study, the KRAS variant, along with other molecular markers like the KRAS and BRAF mutational status, is evaluated in a series of 559 mCRC patients who underwent salvage anti-EGFR MoAbs monotherapy or MoAbs in combination with chemotherapy. The data presented herein clarify the role of the KRAS variant in predicting response to MoAbs therapy. In this patient cohort, as well as in cell lines, that the KRAS variant (G allele) predicts a positive response to MoAbs monotherapy, without any additional benefit of cytotoxic chemotherapy.

The studies presented herein demonstrate a statistically significant improvement in median PFS for all KRAS variant carriers with metastatic colon cancer (and a trend towards improved OS in the double wt patients) who received anti-EGFR moAbs monotherapy. Moreover, a statistically significant was discovered for a favorable prognosis of these patients compared to non-KRAS variant carriers across all cohorts studied in the response to anti-EGFR moAbs, including KRAS or RAF mutant patients. This improved prognosis was not dependent on the addition of chemotherapy, and in fact, KRAS variant (G allele) carriers appeared to have no benefit to chemotherapy in addition to anti-EGFR moAbs therapy. This was in contrast to non-KRAS variant patients, who derived a significant benefit from the addition of chemotherapy to anti-EGFR moAbs across all cohorts, and the addition of chemotherapy brought their prognosis to the same level of KRAS variant allele carriers who received anti-EGFR moAbs monotherapy. Cell lines studies showed the same effect with lack of benefit of combination therapy in KRAS variant cell lines compared to non-variant cell lines. These findings suggest for the first time the KRAS variant allele patients with metastatic colon cancer could and perhaps should avoid the toxic and sometimes deadly affect of chemotherapy treatment, and could be meaningfully treated with anti-EGFR moAbs monotherapy alone.

A population of patients mainly of European origin showed an elevated frequency of the KRAS variant of 19.5%, compared to reported baseline prevalences. While the KRAS variant is found in 6% of the world population, its frequency has been estimated to rise above 10% in healthy Caucasians. Furthermore, the prevalence of the KRAS variant is substantially increased to almost 20% in patients suffering from NSCLC, highlighting an association of increased risk. In the Caucasian mCRC patient population with European descent studied by Graziano et al (Pharmacogenomics J 2010; 10(5):458-464) the KRAS variant (G allele) (incorporating both TG and GG genotypes) was found in 25%, of patients, whereas, in a more heterogeneous population in the study by Zhang et al (Ann Oncol 2011; 22(1):104-109) the frequency of the KRAS variant was 15.3%. Data provided herein did not find that the KRAS variant allele was a risk for developing colon cancer, although the KRAS variant was enriched in patients with Stage IV disease. Together, the evidence indicates that although the KRAS variant (G allele) is not a risk for all types of colon cancer, it is associated with the likelihood of developing advanced and metastatic colon cancer. The KRAS variant predicts a good prognosis in both early stage colon cancer as well as metastatic colon cancer patients when treated with Cetuximab monotherapy. However, the KRAS variant (G allele) may be associated with the development of metastatic disease in colon cancer, which is universally fatal.

A different distribution of the KRAS variant genotypes according to the KRAS and BRAF mutational status was observed in this study with respect to the mCRC patient population compared to prior reports. In this study, the KRAS genotypes were equally distributed among the KRAS wt and mutated groups, but, in the BRAF mutated group, the frequency of the KRAS variant was statistically significantly increased, i.e., twice as high compared to wild type. In the later stages of CRC carcinogenesis, the KRAS variant allele may mediate the selection of less differentiated and more aggressive clones that carry BRAF mutations. Additionally, a selective pressure may favor the development of KRAS or BRAF mutations in the presence of the KRAS variant, depending on exposure to specific therapies. Patients with the KRAS variant (G allele) have a different prognosis when treated with Cetuximab regardless of patients also having a KRAS or a BRAF mutation, suggesting that these groups need re-evaluation for the potential of Cetuximab treatment.

When the survival outcomes were analyzed according to treatment, in the whole and the double wt patient populations treated with anti-EGFR moAbs monotherapy, the KRAS variant genotype carriers had a statistically significantly longer PFS (p=0.019 and p=0.039, respectively). Although, in the whole monotherapy patient population the KRAS variant genotype carriers had a longer OS of 45 weeks compared to 28.85 weeks of the wt carriers, nevertheless this difference did not reach statistical significance. In the double (KRAS and BRAF) wt patient population a trend towards statistical significance (p=0.087) was observed with a longer OS in favor of the KRAS variant carriers (55.43 vs. 35.71 weeks).

Cetuximab/irinotecan treated KRAS mutated patients with the KRAS variant (G-allele) genotype showed a significantly worse PFS of 6.4 weeks compared to 12 weeks in those patients with the LCS6 wt genotype (p=0.037, log-rank test). In our analysis, in the anti-EGFR moAbs-based combination chemotherapy group, where people were treated with a variety of agents, no statistically significant differences were found in PFS or OS in any population between the KRAS variant and wt genotype carriers. There was a trend for worse survival (23 versus 28 weeks) in KRAS variant carriers with KRAS or RAF mutations when they received chemotherapy versus monotherapy, respectively. These findings collectively may indicate that certain chemotherapy in combination with anti-EGFR moAbs-based therapy in KRAS variant carriers is detrimental.

An important step in the development of CRC, among other cancers, is the deregulation of miRNAs. Over the past few years miRNAs have been brought to the central stage of molecular oncology and have substantially changed the way we view and understand gene regulation. The KRAS variant was the first SNP in a miRNA binding site to be implicated in cancer risk. The data presented herein indicate that patients carrying the KRAS variant allele genotype are biologically different then non-variant, or LCS6 wt, patients. Patients carrying the KRAS variant allele genotype have a higher probability of benefit from anti-EGFR moAbs monotherapy as well as a better overall prognosis, without a benefit from the addition of chemotherapy. Because tumors with the KRAS variant induce overexpression of the KRAS pathway, upstream inhibition of this pathway could specifically sensitize these tumors. This mechanism appears to contradict the lack of efficacy of moAbs therapy in tumor acquired KRAS mutant tumors, however, it is possible that the KRAS variant does not induce as high of a level of independent KRAS pathway signaling as tumor acquired KRAS mutations.

KRAS variant tumors derive no benefit from the addition of cytotoxic therapy to moAbs monotherapy. Because the KRAS variant is regulated by the let-7 family of miRNA, and because chemotherapy lowers let-7 levels and allows higher KRAS expression (especially in the presence of the KRAS variant), treatment with chemotherapy may increase activation of this allele, thereby removing the ability of upstream moAbs therapy to overcome KRAS pathway activation. The potential of the 3′ UTR functional variants, including the KRAS variant, to predict altered tumor biology and response to treatment and allow better risk stratification of patients.

### MicroRNA

MicroRNAs (miRNAs) are a novel class of small non-coding RNAs that regulate gene expression by base pairing with sequences within the 3′-untranslated regions (UTR) of target mRNAs, as well as 5′-untranslated regions (UTR) and coding sequence regions, causing mRNA cleavage and/or translational repression (He L, et al. Nature 2005; 435: 828-33; Esquela-Kerscher A. and Slack F J. Nat Rev Cancer 2006; 6: 259-69). miRNAs are misregulated in every cancer studied thus far, including, but not limited to, breast and colorectal cancers, where certain miRNA alterations (and specifically reduced let-7) are found in tumor-initiating cells, suggesting that low let-7 allows self-renewal and proliferation of these cells (Yu F, et al. Cell 2007; 131: 1109-23) and increases cancer risk.

Because miRNAs act as global gene regulators, inherited variations in miRNAs are associated with increased cancer risk. Evidence is quickly growing that polymorphisms disrupting miRNA coding sequences (Hoffman A, et al. Cancer Res 2009; 69: 5970-77) or 3′UTR miRNA binding sites are strong predictors of cancer risk, including, but not limited to, breast and colorectal cancers (Pongsavee M, et al. Genet Test Mol Biomarkers 2009; 13: 307-17; Tchatchou S, et al. Carcinogenesis 2009; 30: 59-64). However, none of the previously identified miRNA-altering polymorphisms has been associated with triple negative breast cancer (TNBC), or with altered gene and/or miRNA expression in tumors.

A novel germline polymorphism (rs61764370) in a let-7 miRNA complementary site within the 3′UTR of the KRAS oncogene was recently identified (International Patent Application No. PCT/US2008/065302, the contents of which are incorporated herein by reference in their entirety), referred to as the “LCS6-SNP’ or the ‘KRAS-variant’.

The KRAS variant is associated with low concentrations of let-7 in tumors and altered KRAS regulation in lung cancer (Chin L, et al. Cancer Res 2008; 68: 8535-40). Moreover, the KRAS variant predicts poor cancer specific outcome in head and neck cancer (Christensen B C, et al. Carcinogenesis 2009; 30: 1003-07) and altered drug response in colon cancer (Graziano F, et al. Pharmacogenomics J 2010; 10: 458-64; Zhang W, et al. Ann Oncol 2011; 22: 104-09). The KRAS variant is also enriched in ovarian cancer and is most frequently associated with patients from families with Hereditary Breast and Ovarian Cancer (HBOC) (Ratner E, et al. Cancer Res 2010; 70: 6509-15). The studies provided herein further assess the role of the KRAS variant in cancer risk and tumor biology.

The data provided herein demonstrate, for example, that a germline polymorphism in the KRAS 3′UTR, known as the KRAS variant', is a genetic marker of an increased risk of developing triple negative breast cancer for premenopausal women. Because study group 1 was small and only assessed in patients with known ER and PR statuses, this association was validated in larger case controls with full receptor status. Most importantly, the data demonstrate that the tumors of patients with triple negative breast cancer (TNBC) who have the KRAS-variant have distinct gene expression patterns compared to other patients without the KRAS-variant, demonstrating that the KRAS-variant drives specific pathways that are known to influence tumor biology and modify tumor development. Thus, the KRAS-variant can classify tumors into meaningful biological subgroups to both predict prognosis as well as direct treatment decisions in the future.

The finding of reduced let-7 expression in TNBC tumors associated with the KRAS-variant is clinically important. KRAS overexpression, through NFKB, can lead to induction of lin-28, a negative regulator of let-7, and, consequently, lowering of let-7 expression (Iliopoulos D, et al. Cell 2009; 139: 1-14; Meylan E, et al. Nature 2009; 462: 104-08; Barbie D, et al. Nature 2009; 462: 108-12). This suggests a potential mechanism whereby let-7 is lowered in pre-malignant tissue, and ultimately, tumors associated with the KRAS-variant. Furthermore, let-7 regulates proliferation of breast like stem cells (Yu F, Yao H, Zhu P, et al. Cell 2007; 131: 1109-23), and low let-7 expression or concentrations allow expansion of this group of cells, thereby increasing breast cancer risk in women with the KRAS-variant. The association of the KRAS variant with TNBC risk only in premenopausal women indicates a meaningful interaction between the KRAS-variant and hormonal exposure.

Although more than half of breast tumors that carry the BRCA1 mutation develop into the triple negative subtype (TNBC) (Atchley D P, et al. J Clin Oncol 2008; 26: 4282-88), BRCA1 mutations are rare, and, thus, account for only about 10-15% of all TNBC cases (Young S R, et al. BMC Cancer 2009; 9: 86; Nanda R, et al. JAMA 2005; 294: 1925-33). The KRAS-variant is found in up to 23% of premenopausal TNBC patients, without an apparent significant enrichment in BRCA mutation carriers from these cohorts or in young ER/PR negative BRCA1 mutation carriers (miRNA profiling, publicly available at www.appliedbiosystems.com/absite/us/en/home/applications-technologies/real-time-per/mirna-profiling.html (accessed Jan. 1, 2008)). The KRAS-variant is associated with a BRCA1 mutant-like gene expression signature, indicating that there may be increased oncogenic risk in the presence of the KRAS variant, high KRAS expression and low B RCA1 expression, either through mutation or other mechanisms.

The KRAS-variant affects the regulation of KRAS expression in vitro and promotion of higher KRAS concentrations (Chin L, et al. Cancer Res 2008; 68: 8535-40). The KRAS oncogene is an important upstream mediator of the MAPK pathway, and its overexpression can result in increased activation of the Raf/MEK/MAPK pathway, thereby promoting tumorigenesis. The studies provided herein demonstrate that patients with the KRAS-variant and TNBC show activation of the MAPK pathway (Table X). Hyperactivation of MAPK in breast cancer cells decreases ER expression leading to an ER-negative phenotype (Atchley D P, et al. J Clin Oncol 2008; 26: 4282-88), which agrees with our finding that the KRAS variant is associated with even lower estrogen signaling in these histologically ER negative tumors. MAPK activation has been implicated in estrogen-independent tumor growth and insensitivity to anti-estrogen treatment (Oh A S, et al. Mol Endocrinol 2001; 15: 1344-59), and might be a mechanism by which the KRAS-variant drives the development of TNBC more than other breast cancer subtypes.

The KRAS-variant is a biomarker of poor outcome in several cancers, including head and neck cancer (Christensen B C, et al. Carcinogenesis 2009; 30: 1003-07). The KRAS-variant is also a biomarker of poor response to targeted therapies in combination with chemotherapy in colon cancer (Graziano F, et al. Pharmacogenomics J 2010; 10: 458-64). The discovery that KRAS-variant positive TNBC patients have a luminal progenitor signature and differential expression of angiogenic and metastatic markers within the signature demonstrates that tumors harboring the KRAS variant are an aggressive sub-group of TNBC.

The study provided herein demonstrates that the KRAS-variant is associated with tumors that maintain unique gene expression patterns. Although work is ongoing, data from these studies provide valuable insight into critical steps and pathways required for transformation and tumor development in these women. These are meaningful steps towards understanding the mechanisms of gain of function miRNA disrupting polymorphisms in cancer biology, which are unique in function from previously discovered genetic markers of cancer risk.

### KRAS Variant

The disclosure is based, in part, upon the unexpected discovery that the presence of a SNP in the 3′ untranslated region (UTR) of KRAS, referred to herein as the “LCS6 SNP” or the “KRAS variant,” which is predictive of an individual's risk of developing cancer and an individual's response to treatment for cancer. The KRAS variant is located in LCS6, the wild type and variant sequence of which is provided below.

The KRAS variant may be represented by one or more of the following sequences. For example, the KRAS variant may be defined by the GenBank accession number rs61764370 and the sequence GTCTCGAACTCCTGACCTCAAGTGATGCACCCACCTTGGCCTCATAAACCTG (SEQ ID NO: 22, in which the SNP is bolded and underlined).

There are three human RAS genes comprising HRAS, KRAS, and NRAS. Each gene comprises multiple miRNA complementary sites in the 3′UTR of their mRNA transcripts. Specifically, each human RAS gene comprises multiple let-7 complementary sites (LCSs). The let-7 family-of-microRNAs (miRNAs) includes global genetic regulators important in controlling lung cancer oncogene expression by binding to the 3′UTRs (untranslated regions) of their target messenger RNAs (mRNAs).

Specifically, the term “let-7 complementary site” is meant to describe any region of a gene or gene transcript that binds a member of the let-7 family of miRNAs. Moreover, this term encompasses those sequences within a gene or gene transcript that are complementary to the sequence of a let-7 family miRNA. The term “complementary” describes a threshold of binding between two sequences wherein a majority of nucleotides in each sequence are capable of binding to a majority of nucleotides within the other sequence in trans.

The Human KRAS 3′ UTR comprises 8 LCSs named LCS1-LCS8, respectively. For the following sequences, thymine (T) may be substituted for uracil (U). LCS 1 comprises the sequence GACAGUGGAAGUUUUUUUUUCCUCG (SEQ ID NO: 1). LCS2 comprises the sequence AUUAGUGUCAUCUUGCCUC (SEQ ID NO: 2). LCS3 comprises the sequence AAUGCCCUACAUCUUAUUUUCCUCA (SEQ ID NO: 3). LCS4 comprises the sequence GGUUCAAGCGAUUCUCGUGCCUCG (SEQ ID NO: 4). LCS5 comprises the sequence GGCUGGUCCGAACUCCUGACCUCA (SEQ ID NO: 5). LCS6 comprises the sequence GAUUCACCCACCUUGGCCUCA (SEQ ID NO: 6). LCS7 comprises the sequence GGGUGUUAAGACUUGACACAGUACCUCG (SEQ ID NO: 7). LCS8 comprises the sequence AGUGCUUAUGAGGGGAUAUUUAGGCCUC (SEQ ID NO: 8).

Human KRAS has two wild type forms, encoded by transcripts a and b, which are provided below as SEQ ID NOs: 9 and 10, respectively. The sequences of each human KRAS transcript, containing the LCS6 SNP, are provided below as SEQ ID NOs: 11 and 12.

Human KRAS, transcript variant a, is encoded by the following mRNA sequence (NCBI Accession No. NM_{—}033360 and SEQ ID NO: 9) (untranslated regions are bolded, LCS6 is underlined):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gagagtggag gatgcttttt atacattggt |
| 661 | gagggagatc cgacaataca gattgaaaaa aatcagcaaa gaagaaaaga ctcctggctg |
| 721 | tgtgaaaatt aaaaaatgca ttataatgta atctgggtgt tgatgatgcc ttctatacat |
| 781 | tagttcgaga aattcgaaaa cataaagaaa agatgagcaa agatggtaaa aagaagaaaa |
| 841 | agaagtcaaa gacaaagtgt gtaattatgt aaatacaatt tgtacttttt tcttaaggca |
| 901 | tactagtaca agtggtaatt tttgtacatt acactaaatt attagcattt gttttagcat |
| 961 | tacctaattt ttttcctgct ccatgcagac tgttagcttt taccttaaat gcttatttta |
| 1021 | aaatgacagt ggaagttttt ttttcctcta agtgccagta ttcccagagt tttggttttt |
| 1081 | gaactagcaa tgcctgtgaa aaagaaactg aatacctaag atttctgtct tggggttttt |
| 1141 | ggtgcatgca gttgattact tcttattttt cttaccaatt gtgaatgttg gtgtgaaaca |
| 1201 | aattaatgaa gcttttgaat catccctatt ctgtgtttta tctagtcaca taaatggatt |
| 1261 | aattactaat ttcagttgag accttctaat tggtttttac tgaaacattg agggaacaca |
| 1321 | aatttatggg cttcctgatg atgattcttc taggcatcat gtcctatagt ttgtcatccc |
| 1381 | tgatgaatgt aaagttacac tgttcacaaa ggttttgtct cctttccact gctattagtc |
| 1441 | atggtcactc tccccaaaat attatatttt ttctataaaa agaaaaaaat ggaaaaaaat |
| 1501 | tacaaggcaa tggaaactat tataaggcca tttccttttc acattagata aattactata |
| 1561 | aagactccta atagcttttc ctgttaaggc agacccagta tgaaatgggg attattatag |
| 1621 | caaccatttt ggggctatat ttacatgcta ctaaattttt ataataattg aaaagatttt |
| 1681 | aacaagtata aaaaattctc ataggaatta aatgtagtct ccctgtgtca gactgctctt |
| 1741 | tcatagtata actttaaatc ttttcttcaa cttgagtctt tgaagatagt tttaattctg |
| 1801 | cttgtgacat taaaagatta tttgggccag ttatagctta ttaggtgttg aagagaccaa |
| 1861 | ggttgcaagg ccaggccctg tgtgaacctt tgagctttca tagagagttt cacagcatgg |
| 1921 | actgtgtccc cacggtcatc cagtgttgtc atgcattggt tagtcaaaat ggggagggac |
| 1981 | tagggcagtt tggatagctc aacaagatac aatctcactc tgtggtggtc ctgctgacaa |
| 2041 | atcaagagca ttgcttttgt ttcttaagaa aacaaactct tttttaaaaa ttacttttaa |
| 2101 | atattaactc aaaagttgag attttggggt ggtggtgtgc caagacatta attttttttt |
| 2161 | taaacaatga agtgaaaaag ttttacaatc tctaggtttg gctagttctc ttaacactgg |
| 2221 | ttaaattaac attgcataaa cacttttcaa gtctgatcca tatttaataa tgctttaaaa |
| 2281 | taaaaataaa aacaatcctt ttgataaatt taaaatgtta cttattttaa aataaatgaa |
| 2341 | gtgagatggc atggtgaggt gaaagtatca ctggactagg aagaaggtga cttaggttct |
| 2401 | agataggtgt cttttaggac tctgattttg aggacatcac ttactatcca tttcttcatg |
| 2461 | ttaaaagaag tcatctcaaa ctcttagttt ttttttttta caactatgta atttatattc |
| 2521 | catttacata aggatacact tatttgtcaa gctcagcaca atctgtaaat ttttaaccta |
| 2581 | tgttacacca tcttcagtgc cagtcttggg caaaattgtg caagaggtga agtttatatt |
| 2641 | tgaatatcca ttctcgtttt aggactcttc ttccatatta gtgtcatctt gcctccctac |
| 2701 | cttccacatg ccccatgact tgatgcagtt ttaatacttg taattcccct aaccataaga |
| 2761 | tttactgctg ctgtggatat ctccatgaag ttttcccact gagtcacatc agaaatgccc |
| 2821 | tacatcttat ttcctcaggg ctcaagagaa tctgacagat accataaagg gatttgacct |
| 2881 | aatcactaat tttcaggtgg tggctgatgc tttgaacatc tctttgctgc ccaatccatt |
| 2941 | agcgacagta ggatttttca aacctggtat gaatagacag aaccctatcc agtggaagga |
| 3001 | gaatttaata aagatagtgc tgaaagaatt ccttaggtaa tctataacta ggactactcc |
| 3061 | tggtaacagt aatacattcc attgttttag taaccagaaa tcttcatgca atgaaaaata |
| 3121 | ctttaattca tgaagcttac tttttttttt tggtgtcaga gtctcgctct tgtcacccag |
| 3181 | gctggaatgc agtggcgcca tctcagctca ctgcaacctc catctcccag gttcaagcga |
| 3241 | ttctcgtgcc tcggcctcct gagtagctgg gattacaggc gtgtgccact acactcaact |
| 3301 | aatttttgta tttttaggag agacggggtt tcaccctgtt ggccaggctg gtctcgaact |
| 3361 | cctgacctca agtgattcac ccaccttggc ctcataaacc tgttttgcag aactcattta |
| 3421 | ttcagcaaat atttattgag tgcctaccag atgccagtca ccgcacaagg cactgggtat |
| 3481 | atggtatccc caaacaagag acataatccc ggtccttagg tagtgctagt gtggtctgta |
| 3541 | atatcttact aaggcctttg gtatacgacc cagagataac acgatgcgta ttttagtttt |
| 3601 | gcaaagaagg ggtttggtct ctgtgccagc tctataattg ttttgctacg attccactga |
| 3661 | aactcttcga tcaagctact ttatgtaaat cacttcattg ttttaaagga ataaacttga |
| 3721 | ttatattgtt tttttatttg gcataactgt gattctttta ggacaattac tgtacacatt |
| 3781 | aaggtgtatg tcagatattc atattgaccc aaatgtgtaa tattccagtt ttctctgcat |
| 3841 | aagtaattaa aatatactta aaaattaata gttttatctg ggtacaaata aacaggtgcc |
| 3901 | tgaactagtt cacagacaag gaaacttcta tgtaaaaatc actatgattt ctgaattgct |
| 3961 | atgtgaaact acagatcttt ggaacactgt ttaggtaggg tgttaagact tacacagtac |
| 4021 | ctcgtttcta cacagagaaa gaaatggcca tacttcagga actgcagtgc ttatgagggg |
| 4081 | atatttaggc ctcttgaatt tttgatgtag atgggcattt ttttaaggta gtggttaatt |
| 4141 | acctttatgt gaactttgaa tggtttaaca aaagatttgt ttttgtagag attttaaagg |
| 4201 | gggagaattc tagaaataaa tgttacctaa ttattacagc cttaaagaca aaaatccttg |
| 4261 | ttgaagtttt tttaaaaaaa gctaaattac atagacttag gcattaacat gtttgtggaa |
| 4321 | gaatatagca gacgtatatt gtatcatttg agtgaatgtt cccaagtagg cattctaggc |
| 4381 | tctatttaac tgagtcacac tgcataggaa tttagaacct aacttttata ggttatcaaa |
| 4441 | actgttgtca ccattgcaca attttgtcct aatatataca tagaaacttt gtggggcatg |
| 4501 | ttaagttaca gtttgcacaa gttcatctca tttgtattcc attgattttt tttttcttct |
| 4561 | aaacattttt tcttcaaaca gtatataact ttttttaggg gatttttttt tagacagcaa |
| 4621 | aaactatctg aagatttcca tttgtcaaaa agtaatgatt tcttgataat tgtgtagtaa |
| 4681 | tgttttttag aacccagcag ttaccttaaa gctgaattta tatttagtaa cttctgtgtt |
| 4741 | aatactggat agcatgaatt ctgcattgag aaactgaata gctgtcataa aatgaaactt |
| 4801 | tctttctaaa gaaagatact cacatgagtt cttgaagaat agtcataact agattaagat |
| 4861 | ctgtgtttta gtttaatagt ttgaagtgcc tgtttgggat aatgataggt aatttagatg |
| 4921 | aatttagggg aaaaaaaagt tatctgcaga tatgttgagg gcccatctct ccccccacac |
| 4981 | ccccacagag ctaactgggt tacagtgttt tatccgaaag tttccaattc cactgtcttg |
| 5041 | tgttttcatg ttgaaaatac ttttgcattt ttcctttgag tgccaatttc ttactagtac |
| 5101 | tatttcttaa tgtaacatgt ttacctggaa tgtattttaa ctatttttgt atagtgtaaa |
| 5161 | ctgaaacatg cacattttgt acattgtgct ttcttttgtg ggacatatgc agtgtgatcc |
| 5221 | agttgttttc catcatttgg ttgcgctgac ctaggaatgt tggtcatatc aaacattaaa |
| 5281 | aatgaccact cttttaattg aaattaactt ttaaatgttt ataggagtat gtgctgtgaa |
| 5341 | gtgatctaaa atttgtaata tttttgtcat gaactgtact actcctaatt attgtaatgt |
| 5401 | aataaaaata gttacagtga caaaaaaaaa aaaaaa |

Human KRAS, transcript variant b, is encoded by the following mRNA sequence (NCBI Accession No. NM_{—}004985 and SEQ ID NO: 10) (untranslated regions are bolded, LCS6 is underlined):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gggtgttgat gatgccttct atacattagt |
| 661 | tcgagaaatt cgaaaacata aagaaaagat gagcaaagat ggtaaaaaga agaaaaagaa |
| 721 | gtcaaagaca aagtgtgtaa ttatgtaaat acaatttgta cttttttctt aaggcatact |
| 781 | agtacaagtg gtaatttttg tacattacac taaattatta gcatttgttt tagcattacc |
| 841 | taattttttt cctgctccat gcagactgtt agcttttacc ttaaatgctt attttaaaat |
| 901 | gacagtggaa gttttttttt cctctaagtg ccagtattcc cagagttttg gtttttgaac |
| 961 | tagcaatgcc tgtgaaaaag aaactgaata cctaagattt ctgtcttggg gtttttggtg |
| 1021 | catgcagttg attacttctt atttttctta ccaattgtga atgttggtgt gaaacaaatt |
| 1081 | aatgaagctt ttgaatcatc cctattctgt gttttatcta gtcacataaa tggattaatt |
| 1141 | actaatttca gttgagacct tctaattggt ttttactgaa acattgaggg aacacaaatt |
| 1201 | tatgggcttc ctgatgatga ttcttctagg catcatgtcc tatagtttgt catccctgat |
| 1261 | gaatgtaaag ttacactgtt cacaaaggtt ttgtctcctt tccactgcta ttagtcatgg |
| 1321 | tcactctccc caaaatatta tattttttct ataaaaagaa aaaaatggaa aaaaattaca |
| 1381 | aggcaatgga aactattata aggccatttc cttttcacat tagataaatt actataaaga |
| 1441 | ctcctaatag cttttcctgt taaggcagac ccagtatgaa atggggatta ttatagcaac |
| 1501 | cattttgggg ctatatttac atgctactaa atttttataa taattgaaaa gattttaaca |
| 1561 | agtataaaaa attctcatag gaattaaatg tagtctccct gtgtcagact gctctttcat |
| 1621 | agtataactt taaatctttt cttcaacttg agtctttgaa gatagtttta attctgcttg |
| 1681 | tgacattaaa agattatttg ggccagttat agcttattag gtgttgaaga gaccaaggtt |
| 1741 | gcaaggccag gccctgtgtg aacctttgag ctttcataga gagtttcaca gcatggactg |
| 1801 | tgtccccacg gtcatccagt gttgtcatgc attggttagt caaaatgggg agggactagg |
| 1861 | gcagtttgga tagctcaaca agatacaatc tcactctgtg gtggtcctgc tgacaaatca |
| 1921 | agagcattgc ttttgtttct taagaaaaca aactcttttt taaaaattac ttttaaatat |
| 1981 | taactcaaaa gttgagattt tggggtggtg gtgtgccaag acattaattt tttttttaaa |
| 2041 | caatgaagtg aaaaagtttt acaatctcta ggtttggcta gttctcttaa cactggttaa |
| 2101 | attaacattg cataaacact tttcaagtct gatccatatt taataatgct ttaaaataaa |
| 2161 | aataaaaaca atccttttga taaatttaaa atgttactta ttttaaaata aatgaagtga |
| 2221 | gatggcatgg tgaggtgaaa gtatcactgg actaggaaga aggtgactta ggttctagat |
| 2281 | aggtgtcttt taggactctg attttgagga catcacttac tatccatttc ttcatgttaa |
| 2341 | aagaagtcat ctcaaactct tagttttttt tttttacaac tatgtaattt atattccatt |
| 2401 | tacataagga tacacttatt tgtcaagctc agcacaatct gtaaattttt aacctatgtt |
| 2461 | acaccatctt cagtgccagt cttgggcaaa attgtgcaag aggtgaagtt tatatttgaa |
| 2521 | tatccattct cgttttagga ctcttcttcc atattagtgt catcttgcct ccctaccttc |
| 2581 | cacatgcccc atgacttgat gcagttttaa tacttgtaat tcccctaacc ataagattta |
| 2641 | ctgctgctgt ggatatctcc atgaagtttt cccactgagt cacatcagaa atgccctaca |
| 2701 | tcttatttcc tcagggctca agagaatctg acagatacca taaagggatt tgacctaatc |
| 2761 | actaattttc aggtggtggc tgatgctttg aacatctctt tgctgcccaa tccattagcg |
| 2821 | acagtaggat ttttcaaacc tggtatgaat agacagaacc ctatccagtg gaaggagaat |
| 2881 | ttaataaaga tagtgctgaa agaattcctt aggtaatcta taactaggac tactcctggt |
| 2941 | aacagtaata cattccattg ttttagtaac cagaaatctt catgcaatga aaaatacttt |
| 3001 | aattcatgaa gcttactttt tttttttggt gtcagagtct cgctcttgtc acccaggctg |
| 3061 | gaatgcagtg gcgccatctc agctcactgc aacctccatc tcccaggttc aagcgattct |
| 3121 | cgtgcctcgg cctcctgagt agctgggatt acaggcgtgt gccactacac tcaactaatt |
| 3181 | tttgtatttt taggagagac ggggtttcac cctgttggcc aggctggtct cgaactcctg |
| 3241 | acctcaagtg attcacccac cttggcctca taaacctgtt ttgcagaact catttattca |
| 3301 | gcaaatattt attgagtgcc taccagatgc cagtcaccgc acaaggcact gggtatatgg |
| 3361 | tatccccaaa caagagacat aatcccggtc cttaggtagt gctagtgtgg tctgtaatat |
| 3421 | cttactaagg cctttggtat acgacccaga gataacacga tgcgtatttt agttttgcaa |
| 3481 | agaaggggtt tggtctctgt gccagctcta taattgtttt gctacgattc cactgaaact |
| 3541 | cttcgatcaa gctactttat gtaaatcact tcattgtttt aaaggaataa acttgattat |
| 3601 | attgtttttt tatttggcat aactgtgatt cttttaggac aattactgta cacattaagg |
| 3661 | tgtatgtcag atattcatat tgacccaaat gtgtaatatt ccagttttct ctgcataagt |
| 3721 | aattaaaata tacttaaaaa ttaatagttt tatctgggta caaataaaca ggtgcctgaa |
| 3781 | ctagttcaca gacaaggaaa cttctatgta aaaatcacta tgatttctga attgctatgt |
| 3841 | gaaactacag atctttggaa cactgtttag gtagggtgtt aagacttaca cagtacctcg |
| 3901 | tttctacaca gagaaagaaa tggccatact tcaggaactg cagtgcttat gaggggatat |
| 3961 | ttaggcctct tgaatttttg atgtagatgg gcattttttt aaggtagtgg ttaattacct |
| 4021 | ttatgtgaac tttgaatggt ttaacaaaag atttgttttt gtagagattt taaaggggga |
| 4081 | gaattctaga aataaatgtt acctaattat tacagcctta aagacaaaaa tccttgttga |
| 4141 | agttttttta aaaaaagcta aattacatag acttaggcat taacatgttt gtggaagaat |
| 4201 | atagcagacg tatattgtat catttgagtg aatgttccca agtaggcatt ctaggctcta |
| 4261 | tttaactgag tcacactgca taggaattta gaacctaact tttataggtt atcaaaactg |
| 4321 | ttgtcaccat tgcacaattt tgtcctaata tatacataga aactttgtgg ggcatgttaa |
| 4381 | gttacagttt gcacaagttc atctcatttg tattccattg attttttttt tcttctaaac |
| 4441 | attttttctt caaacagtat ataacttttt ttaggggatt tttttttaga cagcaaaaac |
| 4501 | tatctgaaga tttccatttg tcaaaaagta atgatttctt gataattgtg tagtaatgtt |
| 4561 | ttttagaacc cagcagttac cttaaagctg aatttatatt tagtaacttc tgtgttaata |
| 4621 | ctggatagca tgaattctgc attgagaaac tgaatagctg tcataaaatg aaactttctt |
| 4681 | tctaaagaaa gatactcaca tgagttcttg aagaatagtc ataactagat taagatctgt |
| 4741 | gttttagttt aatagtttga agtgcctgtt tgggataatg ataggtaatt tagatgaatt |
| 4801 | taggggaaaa aaaagttatc tgcagatatg ttgagggccc atctctcccc ccacaccccc |
| 4861 | acagagctaa ctgggttaca gtgttttatc cgaaagtttc caattccact gtcttgtgtt |
| 4921 | ttcatgttga aaatactttt gcatttttcc tttgagtgcc aatttcttac tagtactatt |
| 4981 | tcttaatgta acatgtttac ctggaatgta ttttaactat ttttgtatag tgtaaactga |
| 5041 | aacatgcaca ttttgtacat tgtgctttct tttgtgggac atatgcagtg tgatccagtt |
| 5101 | gttttccatc atttggttgc gctgacctag gaatgttggt catatcaaac attaaaaatg |
| 5161 | accactcttt taattgaaat taacttttaa atgtttatag gagtatgtgc tgtgaagtga |
| 5221 | tctaaaattt gtaatatttt tgtcatgaac tgtactactc ctaattattg taatgtaata |
| 5281 | aaaatagtta cagtgacaaa aaaaaaaaaa aa |

Human KRAS, transcript variant a, comprising the LCS6 SNP, is encoded by the following mRNA sequence (SEQ ID NO: 11) (untranslated regions are bolded, LCS6 is underlined, SNP is capitalized):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gagagtggag gatgcttttt atacattggt |
| 661 | gagggagatc cgacaataca gattgaaaaa aatcagcaaa gaagaaaaga ctcctggctg |
| 721 | tgtgaaaatt aaaaaatgca ttataatgta atctgggtgt tgatgatgcc ttctatacat |
| 781 | tagttcgaga aattcgaaaa cataaagaaa agatgagcaa agatggtaaa aagaagaaaa |
| 841 | agaagtcaaa gacaaagtgt gtaattatgt aaatacaatt tgtacttttt tcttaaggca |
| 901 | tactagtaca agtggtaatt tttgtacatt acactaaatt attagcattt gttttagcat |
| 961 | tacctaattt ttttcctgct ccatgcagac tgttagcttt taccttaaat gcttatttta |
| 1021 | aaatgacagt ggaagttttt ttttcctcta agtgccagta ttcccagagt tttggttttt |
| 1081 | gaactagcaa tgcctgtgaa aaagaaactg aatacctaag atttctgtct tggggttttt |
| 1141 | ggtgcatgca gttgattact tcttattttt cttaccaatt gtgaatgttg gtgtgaaaca |
| 1201 | aattaatgaa gcttttgaat catccctatt ctgtgtttta tctagtcaca taaatggatt |
| 1261 | aattactaat ttcagttgag accttctaat tggtttttac tgaaacattg agggaacaca |
| 1321 | aatttatggg cttcctgatg atgattcttc taggcatcat gtcctatagt ttgtcatccc |
| 1381 | tgatgaatgt aaagttacac tgttcacaaa ggttttgtct cctttccact gctattagtc |
| 1441 | atggtcactc tccccaaaat attatatttt ttctataaaa agaaaaaaat ggaaaaaaat |
| 1501 | tacaaggcaa tggaaactat tataaggcca tttccttttc acattagata aattactata |
| 1561 | aagactccta atagcttttc ctgttaaggc agacccagta tgaaatgggg attattatag |
| 1621 | caaccatttt ggggctatat ttacatgcta ctaaattttt ataataattg aaaagatttt |
| 1681 | aacaagtata aaaaattctc ataggaatta aatgtagtct ccctgtgtca gactgctctt |
| 1741 | tcatagtata actttaaatc ttttcttcaa cttgagtctt tgaagatagt tttaattctg |
| 1801 | cttgtgacat taaaagatta tttgggccag ttatagctta ttaggtgttg aagagaccaa |
| 1861 | ggttgcaagg ccaggccctg tgtgaacctt tgagctttca tagagagttt cacagcatgg |
| 1921 | actgtgtccc cacggtcatc cagtgttgtc atgcattggt tagtcaaaat ggggagggac |
| 1981 | tagggcagtt tggatagctc aacaagatac aatctcactc tgtggtggtc ctgctgacaa |
| 2041 | atcaagagca ttgcttttgt ttcttaagaa aacaaactct tttttaaaaa ttacttttaa |
| 2101 | atattaactc aaaagttgag attttggggt ggtggtgtgc caagacatta attttttttt |
| 2161 | taaacaatga agtgaaaaag ttttacaatc tctaggtttg gctagttctc ttaacactgg |
| 2221 | ttaaattaac attgcataaa cacttttcaa gtctgatcca tatttaataa tgctttaaaa |
| 2281 | taaaaataaa aacaatcctt ttgataaatt taaaatgtta cttattttaa aataaatgaa |
| 2341 | gtgagatggc atggtgaggt gaaagtatca ctggactagg aagaaggtga cttaggttct |
| 2401 | agataggtgt cttttaggac tctgattttg aggacatcac ttactatcca tttcttcatg |
| 2461 | ttaaaagaag tcatctcaaa ctcttagttt ttttttttta caactatgta atttatattc |
| 2521 | catttacata aggatacact tatttgtcaa gctcagcaca atctgtaaat ttttaaccta |
| 2581 | tgttacacca tcttcagtgc cagtcttggg caaaattgtg caagaggtga agtttatatt |
| 2641 | tgaatatcca ttctcgtttt aggactcttc ttccatatta gtgtcatctt gcctccctac |
| 2701 | cttccacatg ccccatgact tgatgcagtt ttaatacttg taattcccct aaccataaga |
| 2761 | tttactgctg ctgtggatat ctccatgaag ttttcccact gagtcacatc agaaatgccc |
| 2821 | tacatcttat ttcctcaggg ctcaagagaa tctgacagat accataaagg gatttgacct |
| 2881 | aatcactaat tttcaggtgg tggctgatgc tttgaacatc tctttgctgc ccaatccatt |
| 2941 | agcgacagta ggatttttca aacctggtat gaatagacag aaccctatcc agtggaagga |
| 3001 | gaatttaata aagatagtgc tgaaagaatt ccttaggtaa tctataacta ggactactcc |
| 3061 | tggtaacagt aatacattcc attgttttag taaccagaaa tcttcatgca atgaaaaata |
| 3121 | ctttaattca tgaagcttac tttttttttt tggtgtcaga gtctcgctct tgtcacccag |
| 3181 | gctggaatgc agtggcgcca tctcagctca ctgcaacctc catctcccag gttcaagcga |
| 3241 | ttctcgtgcc tcggcctcct gagtagctgg gattacaggc gtgtgccact acactcaact |
| 3301 | aatttttgta tttttaggag agacggggtt tcaccctgtt ggccaggctg gtctcgaact |
| 3361 | cctgacctca agtgatGcac ccaccttggc ctcataaacc tgttttgcag aactcattta |
| 3421 | ttcagcaaat atttattgag tgcctaccag atgccagtca ccgcacaagg cactgggtat |
| 3481 | atggtatccc caaacaagag acataatccc ggtccttagg tagtgctagt gtggtctgta |
| 3541 | atatcttact aaggcctttg gtatacgacc cagagataac acgatgcgta ttttagtttt |
| 3601 | gcaaagaagg ggtttggtct ctgtgccagc tctataattg ttttgctacg attccactga |
| 3661 | aactcttcga tcaagctact ttatgtaaat cacttcattg ttttaaagga ataaacttga |
| 3721 | ttatattgtt tttttatttg gcataactgt gattctttta ggacaattac tgtacacatt |
| 3781 | aaggtgtatg tcagatattc atattgaccc aaatgtgtaa tattccagtt ttctctgcat |
| 3841 | aagtaattaa aatatactta aaaattaata gttttatctg ggtacaaata aacaggtgcc |
| 3901 | tgaactagtt cacagacaag gaaacttcta tgtaaaaatc actatgattt ctgaattgct |
| 3961 | atgtgaaact acagatcttt ggaacactgt ttaggtaggg tgttaagact tacacagtac |
| 4021 | ctcgtttcta cacagagaaa gaaatggcca tacttcagga actgcagtgc ttatgagggg |
| 4081 | atatttaggc ctcttgaatt tttgatgtag atgggcattt ttttaaggta gtggttaatt |
| 4141 | acctttatgt gaactttgaa tggtttaaca aaagatttgt ttttgtagag attttaaagg |
| 4201 | gggagaattc tagaaataaa tgttacctaa ttattacagc cttaaagaca aaaatccttg |
| 4261 | ttgaagtttt tttaaaaaaa gctaaattac atagacttag gcattaacat gtttgtggaa |
| 4321 | gaatatagca gacgtatatt gtatcatttg agtgaatgtt cccaagtagg cattctaggc |
| 4381 | tctatttaac tgagtcacac tgcataggaa tttagaacct aacttttata ggttatcaaa |
| 4441 | actgttgtca ccattgcaca attttgtcct aatatataca tagaaacttt gtggggcatg |
| 4501 | ttaagttaca gtttgcacaa gttcatctca tttgtattcc attgattttt tttttcttct |
| 4561 | aaacattttt tcttcaaaca gtatataact ttttttaggg gatttttttt tagacagcaa |
| 4621 | aaactatctg aagatttcca tttgtcaaaa agtaatgatt tcttgataat tgtgtagtaa |
| 4681 | tgttttttag aacccagcag ttaccttaaa gctgaattta tatttagtaa cttctgtgtt |
| 4741 | aatactggat agcatgaatt ctgcattgag aaactgaata gctgtcataa aatgaaactt |
| 4801 | tctttctaaa gaaagatact cacatgagtt cttgaagaat agtcataact agattaagat |
| 4861 | ctgtgtttta gtttaatagt ttgaagtgcc tgtttgggat aatgataggt aatttagatg |
| 4921 | aatttagggg aaaaaaaagt tatctgcaga tatgttgagg gcccatctct ccccccacac |
| 4981 | ccccacagag ctaactgggt tacagtgttt tatccgaaag tttccaattc cactgtcttg |
| 5041 | tgttttcatg ttgaaaatac ttttgcattt ttcctttgag tgccaatttc ttactagtac |
| 5101 | tatttcttaa tgtaacatgt ttacctggaa tgtattttaa ctatttttgt atagtgtaaa |
| 5161 | ctgaaacatg cacattttgt acattgtgct ttcttttgtg ggacatatgc agtgtgatcc |
| 5221 | agttgttttc catcatttgg ttgcgctgac ctaggaatgt tggtcatatc aaacattaaa |
| 5281 | aatgaccact cttttaattg aaattaactt ttaaatgttt ataggagtat gtgctgtgaa |
| 5341 | gtgatctaaa atttgtaata tttttgtcat gaactgtact actcctaatt attgtaatgt |
| 5401 | aataaaaata gttacagtga caaaaaaaaa aaaaaa |

Human KRAS, transcript variant b, comprising the LCS6 SNP, is encoded by the following mRNA sequence (SEQ ID NO: 12) (untranslated regions are bolded, LCS6 is underlined, SNP is capitalized):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gggtgttgat gatgccttct atacattagt |
| 661 | tcgagaaatt cgaaaacata aagaaaagat gagcaaagat ggtaaaaaga agaaaaagaa |
| 721 | gtcaaagaca aagtgtgtaa ttatgtaaat acaatttgta cttttttctt aaggcatact |
| 781 | agtacaagtg gtaatttttg tacattacac taaattatta gcatttgttt tagcattacc |
| 841 | taattttttt cctgctccat gcagactgtt agcttttacc ttaaatgctt attttaaaat |
| 901 | gacagtggaa gttttttttt cctctaagtg ccagtattcc cagagttttg gtttttgaac |
| 961 | tagcaatgcc tgtgaaaaag aaactgaata cctaagattt ctgtcttggg gtttttggtg |
| 1021 | catgcagttg attacttctt atttttctta ccaattgtga atgttggtgt gaaacaaatt |
| 1081 | aatgaagctt ttgaatcatc cctattctgt gttttatcta gtcacataaa tggattaatt |
| 1141 | actaatttca gttgagacct tctaattggt ttttactgaa acattgaggg aacacaaatt |
| 1201 | tatgggcttc ctgatgatga ttcttctagg catcatgtcc tatagtttgt catccctgat |
| 1261 | gaatgtaaag ttacactgtt cacaaaggtt ttgtctcctt tccactgcta ttagtcatgg |
| 1321 | tcactctccc caaaatatta tattttttct ataaaaagaa aaaaatggaa aaaaattaca |
| 1381 | aggcaatgga aactattata aggccatttc cttttcacat tagataaatt actataaaga |
| 1441 | ctcctaatag cttttcctgt taaggcagac ccagtatgaa atggggatta ttatagcaac |
| 1501 | cattttgggg ctatatttac atgctactaa atttttataa taattgaaaa gattttaaca |
| 1561 | agtataaaaa attctcatag gaattaaatg tagtctccct gtgtcagact gctctttcat |
| 1621 | agtataactt taaatctttt cttcaacttg agtctttgaa gatagtttta attctgcttg |
| 1681 | tgacattaaa agattatttg ggccagttat agcttattag gtgttgaaga gaccaaggtt |
| 1741 | gcaaggccag gccctgtgtg aacctttgag ctttcataga gagtttcaca gcatggactg |
| 1801 | tgtccccacg gtcatccagt gttgtcatgc attggttagt caaaatgggg agggactagg |
| 1861 | gcagtttgga tagctcaaca agatacaatc tcactctgtg gtggtcctgc tgacaaatca |
| 1921 | agagcattgc ttttgtttct taagaaaaca aactcttttt taaaaattac ttttaaatat |
| 1981 | taactcaaaa gttgagattt tggggtggtg gtgtgccaag acattaattt tttttttaaa |
| 2041 | caatgaagtg aaaaagtttt acaatctcta ggtttggcta gttctcttaa cactggttaa |
| 2101 | attaacattg cataaacact tttcaagtct gatccatatt taataatgct ttaaaataaa |
| 2161 | aataaaaaca atccttttga taaatttaaa atgttactta ttttaaaata aatgaagtga |
| 2221 | gatggcatgg tgaggtgaaa gtatcactgg actaggaaga aggtgactta ggttctagat |
| 2281 | aggtgtcttt taggactctg attttgagga catcacttac tatccatttc ttcatgttaa |
| 2341 | aagaagtcat ctcaaactct tagttttttt tttttacaac tatgtaattt atattccatt |
| 2401 | tacataagga tacacttatt tgtcaagctc agcacaatct gtaaattttt aacctatgtt |
| 2461 | acaccatctt cagtgccagt cttgggcaaa attgtgcaag aggtgaagtt tatatttgaa |
| 2521 | tatccattct cgttttagga ctcttcttcc atattagtgt catcttgcct ccctaccttc |
| 2581 | cacatgcccc atgacttgat gcagttttaa tacttgtaat tcccctaacc ataagattta |
| 2641 | ctgctgctgt ggatatctcc atgaagtttt cccactgagt cacatcagaa atgccctaca |
| 2701 | tcttatttcc tcagggctca agagaatctg acagatacca taaagggatt tgacctaatc |
| 2761 | actaattttc aggtggtggc tgatgctttg aacatctctt tgctgcccaa tccattagcg |
| 2821 | acagtaggat ttttcaaacc tggtatgaat agacagaacc ctatccagtg gaaggagaat |
| 2881 | ttaataaaga tagtgctgaa agaattcctt aggtaatcta taactaggac tactcctggt |
| 2941 | aacagtaata cattccattg ttttagtaac cagaaatctt catgcaatga aaaatacttt |
| 3001 | aattcatgaa gcttactttt tttttttggt gtcagagtct cgctcttgtc acccaggctg |
| 3061 | gaatgcagtg gcgccatctc agctcactgc aacctccatc tcccaggttc aagcgattct |
| 3121 | cgtgcctcgg cctcctgagt agctgggatt acaggcgtgt gccactacac tcaactaatt |
| 3181 | tttgtatttt taggagagac ggggtttcac cctgttggcc aggctggtct cgaactcctg |
| 3241 | acctcaagtg atGcacccac cttggcctca taaacctgtt ttgcagaact catttattca |
| 3301 | gcaaatattt attgagtgcc taccagatgc cagtcaccgc acaaggcact gggtatatgg |
| 3361 | tatccccaaa caagagacat aatcccggtc cttaggtagt gctagtgtgg tctgtaatat |
| 3421 | cttactaagg cctttggtat acgacccaga gataacacga tgcgtatttt agttttgcaa |
| 3481 | agaaggggtt tggtctctgt gccagctcta taattgtttt gctacgattc cactgaaact |
| 3541 | cttcgatcaa gctactttat gtaaatcact tcattgtttt aaaggaataa acttgattat |
| 3601 | attgtttttt tatttggcat aactgtgatt cttttaggac aattactgta cacattaagg |
| 3661 | tgtatgtcag atattcatat tgacccaaat gtgtaatatt ccagttttct ctgcataagt |
| 3721 | aattaaaata tacttaaaaa ttaatagttt tatctgggta caaataaaca ggtgcctgaa |
| 3781 | ctagttcaca gacaaggaaa cttctatgta aaaatcacta tgatttctga attgctatgt |
| 3841 | gaaactacag atctttggaa cactgtttag gtagggtgtt aagacttaca cagtacctcg |
| 3901 | tttctacaca gagaaagaaa tggccatact tcaggaactg cagtgcttat gaggggatat |
| 3961 | ttaggcctct tgaatttttg atgtagatgg gcattttttt aaggtagtgg ttaattacct |
| 4021 | ttatgtgaac tttgaatggt ttaacaaaag atttgttttt gtagagattt taaaggggga |
| 4081 | gaattctaga aataaatgtt acctaattat tacagcctta aagacaaaaa tccttgttga |
| 4141 | agttttttta aaaaaagcta aattacatag acttaggcat taacatgttt gtggaagaat |
| 4201 | atagcagacg tatattgtat catttgagtg aatgttccca agtaggcatt ctaggctcta |
| 4261 | tttaactgag tcacactgca taggaattta gaacctaact tttataggtt atcaaaactg |
| 4321 | ttgtcaccat tgcacaattt tgtcctaata tatacataga aactttgtgg ggcatgttaa |
| 4381 | gttacagttt gcacaagttc atctcatttg tattccattg attttttttt tcttctaaac |
| 4441 | attttttctt caaacagtat ataacttttt ttaggggatt tttttttaga cagcaaaaac |
| 4501 | tatctgaaga tttccatttg tcaaaaagta atgatttctt gataattgtg tagtaatgtt |
| 4561 | ttttagaacc cagcagttac cttaaagctg aatttatatt tagtaacttc tgtgttaata |
| 4621 | ctggatagca tgaattctgc attgagaaac tgaatagctg tcataaaatg aaactttctt |
| 4681 | tctaaagaaa gatactcaca tgagttcttg aagaatagtc ataactagat taagatctgt |
| 4741 | gttttagttt aatagtttga agtgcctgtt tgggataatg ataggtaatt tagatgaatt |
| 4801 | taggggaaaa aaaagttatc tgcagatatg ttgagggccc atctctcccc ccacaccccc |
| 4861 | acagagctaa ctgggttaca gtgttttatc cgaaagtttc caattccact gtcttgtgtt |
| 4921 | ttcatgttga aaatactttt gcatttttcc tttgagtgcc aatttcttac tagtactatt |
| 4981 | tcttaatgta acatgtttac ctggaatgta ttttaactat ttttgtatag tgtaaactga |
| 5041 | aacatgcaca ttttgtacat tgtgctttct tttgtgggac atatgcagtg tgatccagtt |
| 5101 | gttttccatc atttggttgc gctgacctag gaatgttggt catatcaaac attaaaaatg |
| 5161 | accactcttt taattgaaat taacttttaa atgtttatag gagtatgtgc tgtgaagtga |
| 5221 | tctaaaattt gtaatatttt tgtcatgaac tgtactactc ctaattattg taatgtaata |
| 5281 | aaaatagtta cagtgacaaa aaaaaaaaaa aa |

The KRAS variant is the result of a substitution of a G for a U at position 4 of SEQ ID NO: 6 of LCS6. This KRAS variant comprises the sequence GAUGCACCCACCUUGGCCUCA (SNP bolded for emphasis) (SEQ ID NO: 13).

The KRAS variant leads to altered KRAS expression by disrupting the miRNA regulation of a KRAS. The identification and characterization of the KRAS variant is further described in International Application No. PCT/US08/65302 (WO 2008/151004), the contents of which are incorporated by reference in their entirety.

Let-7 Family miRNAs

Expression of let-7 family miRNAs is increased in cells that carry the KRAS variant. Interestingly, the let-7 family of miRNAs bind to the let-7 complementary site in which the KRAS variant in located. The presence of the KRAS variant interferes with let-7 binding to KRAS. By interfering, the KRAS variant either induces let-7 to bind more or less tightly to LCS6 of KRAS. It was discovered that cells containing the KRAS variant have lower levels of KRAS mRNA compared to wild type cells, and increased levels of the KRAS protein. Thus, while not wishing to be bound by theory, the presence of the KRAS variant within cells may interfere with the ability of let-7 to bind to KRAS and inhibit protein translation, allowing higher KRAS protein levels.

The presence of the KRAS-Variant in triple negative breast cancer is also associated with significantly lower levels of let-7 miRNAs. For instance, let-7 miRNA expression is decreased by 2-fold (2×), 3-fold (3×), 4-fold (4×), 5-fold (5×), 6-fold (6×), 7-fold (7×), 8-fold (8×), 9-fold (9×), 10-fold (10×), 20-fold (20×), 50-fold (50×), 100-fold (100×), 200-fold (200×), 500-fold (500×), 1000-fold (1000×), or any multiplier in between. Alternatively, or in addition, the statistically significant difference between the reduction of let-7 miRNA expression in a cell obtained from a subject who has triple negative breast cancer compared to the level of let-7 miRNA expression in a cell obtained from a subject who does not have triple negative breast cancer (i.e. a normal or control cell) is exemplified by a p-value of less than 0.05, preferably, a p-value of less than 0.01, or most preferably, a p-value of less than 0.001. The level of let-7 miRNA expression present in a cell obtained from a subject who has triple negative breast cancer may also be compared to a known standard level in the art. Moreover, the level of let-7 expression may be compared between an affected cell and an unaffected cell within a subject who has breast cancer or, specifically triple negative breast cancer, wherein the unaffected cell serves as an internal control.

Exemplary let-7 miRNAs include, but are not limited to, let-7a (let-7a-1, let-7a-2, let-7a-3), let-7b, let-7c, let-7d, let-7e, let-7f (let-7f-1 and let-7f-2), let-7g, and let-7i. For the following sequences, thymine (T) may be substituted for uracil (U). let-7a comprises the sequence UUGAUAUGUUGGAUGAUGGAGU (SEQ ID NO: 14). let-7b comprises the sequence UUGGUGUGUUGGAUGAUGGAGU (SEQ ID NO: 15). let-7c comprises the sequence UUGGUAUGUUGGAUGAUGGAGU (SEQ ID NO: 16). let-7d comprises the sequence UGAUACGUUGGAUGAUGGAGA (SEQ ID NO: 17). let-7e comprises the sequence UAUAUGUUGGAGGAUGGAGU (SEQ ID NO: 18). let-7f comprises the sequence UUGAUAUGUUAGAUGAUGGAGU (SEQ ID NO: 19). let-7g comprises the sequence GACAUGUUUGAUGAUGGAGU (SEQ ID NO: 20). let-7i comprises the sequence UGUCGUGUUUGUUGAUGGAGU (SEQ ID NO: 21).

Sequences of additional let-7 family members are publicly available from miRBase at (www.mirbase.org).

### Therapeutic Methods

Identification of the KRAS variant mutation indicates an increased risk of developing triple negative breast cancer. “Risk” in the context of the present disclosure, relates to the probability that an event will occur over a specific time period, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1-p) where p is the probability of event and (1-p) is the probability of no event) to no-conversion.

“Risk evaluation,” or “evaluation of risk” in the context of the present disclosure encompasses making a prediction of the probability, odds, or likelihood that an event or disease state may occur, the rate of occurrence of the event or conversion from one disease state to another, i.e., from a primary tumor to a metastatic tumor or to one at risk of developing a metastatic, or from at risk of a primary metastatic event to a secondary metastatic event or from at risk of a developing a primary tumor of one type to developing a one or more primary tumors of a different type. Risk evaluation can also comprise prediction of future clinical parameters, traditional laboratory risk factor values, or other indices of cancer, either in absolute or relative terms in reference to a previously measured population.

An “increased risk” is meant to describe an increased probably that an individual who carries the KRAS variant will develop or has developed cancer, when compared to an individual who does not carry the KRAS variant. In certain embodiments, a KRAS variant carrier is 1.5×, 2×, 2.5×, 3×, 3.5×, 4×, 4.5×, 5×, 5.5×, 6×, 6.5×, 7×, 7.5×, 8×, 8.5×, 9×, 9.5×, 10×, 20×, 30×, 40×, 50×, 60×, 70×, 80×, 90×, or 100× more likely to develop or have cancer than an individual who does not carry the KRAS variant.

By poor prognosis is meant that the probability of the individual surviving the development of a particularly aggressive, high-risk, severe, or inherited form of cancer (e.g., triple negative breast cancer), or that the probability of surviving the development or progression of an aggressive, high-risk, severe, or inherited form is less than the probability of surviving the development or progression of a more benign form.

Poor prognosis is also meant to describe a less satisfactory recovery, longer recovery period, more invasive or high-risk therapeutic regime, or an increased probability of reoccurrence of cancer or a metastasis thereof. For example, triple negative breast cancer or a metastasis thereof is correlated with the worst prognosis of breast cancer subtypes, resulting in a poor prognosis for the subject.

The terms subject, patient, and individual are used interchangeably throughout the description. A subject is preferably a mammal. The mammal can be a human, non-human primate, mouse, rat, dog, cat, horse, or cow, but are not limited to these examples. A subject is male or female. A subject may not have been previously diagnosed as having cancer, a particular type of cancer (e.g., breast cancer), or a subtype of cancer (e.g., triple negative breast cancer as a subtype of breast cancer). The subject may exhibit one or more risk factors for cancer, a particular type of cancer (e.g., breast cancer), or a subtype of cancer (e.g., triple negative breast cancer as a subtype of breast cancer). Alternatively, the subject does not exhibit a risk factor for cancer, a particular type of cancer (e.g., breast cancer), or a subtype of cancer (e.g., triple negative breast cancer as a subtype of breast cancer).

Breast cancer, including triple negative breast cancer, risk factors include, but are not limited to, the presence of the KRAS variant; being female, aging, obesity, lack of childbearing or breastfeeding, higher hormone levels, smoking, exposure to radiation, personal history of breast cancer, family history of breast cancer, and particular breast changes (e.g. those changes associated with fibrocystic conditions, including, but not limited to, Atypical hyperplasia and lobular carcinoma in situ). Exemplary protective measures against the development of triple negative breast cancer, include, but not limited to, regular exercise, avoiding environmental triggers (e.g. smoking, drinking, high fat diet leading to obesity, radiation exposure through occupation), choosing to breastfeed children, and, for those at the most severe risk, prophylactic bilateral mastectomy. Subjects of the disclosure may present one or more risk factors that may further be mitigated or modified by a protective measure.

The methods described herein provide for obtaining a sample from a subject. The sample can be any tissue or fluid that contains nucleic acids. Various embodiments include, but are not limited to, paraffin imbedded tissue, frozen tissue, surgical fine needle aspirations, and cells of the breast (including cells harvested from a duct, a lobule, or connective tissue), a lymph node (including a sentinel or axillary node), a thoracic or abdominal muscle or connective tissue, an organ (including any potential deposit site for a potential metastatic cell, such as the brain, liver, kidney, stomach, intestines, bone marrow, pancreas, colon, or lung). Other embodiments include fluid samples such as blood, plasma, serum, lymph fluid, ascites, serous fluid, and urine.

### SNP Genotyping Methods

The KRAS variant is a single nucleotide polymorphism that occurs within the 3′ UTR of the human KRAS gene. Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different SNP sites, which is generally due to the physical proximity of the two loci along a chromosome. LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD.

For screening individuals for genetic disorders (e.g. prognostic or risk) purposes, if a particular SNP site is found to be useful for screening a disorder, then the skilled artisan would recognize that other SNP sites which are in LD with this SNP site would also be useful for screening the condition. Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome.

For screening applications, polymorphisms (e.g., SNPs and/or haplotypes) that are not the actual disease-causing (causative) polymorphisms, but are in LD with such causative polymorphisms, are also useful. In such instances, the genotype of the polymorphism(s) that is/are in LD with the causative polymorphism is predictive of the genotype of the causative polymorphism and, consequently, predictive of the phenotype (e.g., disease) that is influenced by the causative SNP(s). Thus, polymorphic markers that are in LD with causative polymorphisms are useful as markers, and are particularly useful when the actual causative polymorphism(s) is/are unknown.

Linkage disequilibrium in the human genome is reviewed in: Wall et al., “Haplotype blocks and linkage disequilibrium in the human genome”, Nat Rev Genet. 2003 August; 4(8):587-97; Gamer et al., “On selecting markers for association studies: patterns of linkage disequilibrium between two and three diallelic loci”, Genet Epidemiol. 2003 January; 24(1):57-67; Ardlie et al., “Patterns of linkage disequilibrium in the human genome”, Nat Rev Genet. 2002 April; 3(4):299-309 (erratum in Nat Rev Genet. 2002 July; 3(7):566); and Remm et al., “High-density genotyping and linkage disequilibrium in the human genome using chromosome 22 as a model”; Curr Opin Chem. Biol. 2002 February; 6(1):24-30.

The screening techniques of the present disclosure may employ a variety of methodologies to determine whether a test subject has a SNP or a SNP pattern associated with an increased or decreased risk of developing a detectable trait or whether the individual suffers from a detectable trait as a result of a particular polymorphism/mutation, including, for example, methods which enable the analysis of individual chromosomes for haplotyping, family studies, single sperm DNA analysis, or somatic hybrids. The trait analyzed using the diagnostics of the disclosure may be any detectable trait that is commonly observed in pathologies and disorders.

The process of determining which specific nucleotide (i.e., allele) is present at each of one or more SNP positions, such as a SNP position in a nucleic acid molecule disclosed in SEQ ID NO: 11, 12, 13 or 22, is referred to as SNP genotyping. The present disclosure provides methods of SNP genotyping, such as for use in screening for a variety of disorders, or determining predisposition thereto, or determining responsiveness to a form of treatment, or prognosis, or in genome mapping or SNP association analysis, etc.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput”, Pharmacogenomics J. 2003; 3(2):77-96; Kwok et al., “Detection of single nucleotide polymorphisms”, Curr Issues Mol. Biol. 2003 April; 5(2):43-60; Shi, “Technologies for individual genotyping: detection of genetic polymorphisms in drug targets and disease genes”, Am J Pharmacogenomics. 2002; 2(3):197-205; and Kwok, “Methods for genotyping single nucleotide polymorphisms”, Annu Rev Genomics Hum Genet. 2001; 2: 235-58. Exemplary techniques for high-throughput SNP genotyping are described in Marnellos, “High-throughput SNP analysis for genetic association studies”, Curr Opin Drug Discov Devel. 2003 May; 6(3):317-21. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Various methods for detecting polymorphisms include, but are not limited to, methods in which protection from cleavage agents is used to detect mismatched bases in RNA/RNA or RNA/DNA duplexes (Myers et al., Science 230:1242 (1985); Cotton et al., PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285:125-144 (1993); and Hayashi et al., Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations can also be assessed by nuclease protection assays such as RNase and SI protection or chemical cleavage methods.

In a preferred embodiment, SNP genotyping is performed using the TaqMan assay, which is also known as the 5′ nuclease assay (U.S. Pat. Nos. 5,210,015 and 5,538,848). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present disclosure are useful in prognostic assays for a variety of disorders including cancer, and can be readily incorporated into a kit format. The present disclosure also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

The identity of polymorphisms may also be determined using a mismatch detection technique, including but not limited to the RNase protection method using riboprobes (Winter et al., Proc. Natl. Acad. Sci. USA 82:7575, 1985; Meyers et al., Science 230:1242, 1985) and proteins which recognize nucleotide mismatches, such as the E. coli mutS protein (Modrich, P. Ann. Rev. Genet. 25:229-253, 1991). Alternatively, variant alleles can be identified by single strand conformation polymorphism (SSCP) analysis (Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (Wartell et al., Nuci. Acids Res. 18:2699-2706, 1990; Sheffield et al., Proc. Natl. Acad. Sci. USA 86:232-236, 1989).

A polymerase-mediated primer extension method may also be used to identify the polymorphism(s). Several such methods have been described in the patent and scientific literature and include the “Genetic Bit Analysis” method (WO92/15712) and the ligase/polymerase mediated genetic bit analysis (U.S. Pat. No. 5,679,524). Related methods are disclosed in WO91/02087, WO90/09455, WO95/17676, U.S. Pat. Nos. 5,302,509, and 5,945,283. Extended primers containing a polymorphism may be detected by mass spectrometry as described in U.S. Pat. No. 5,605,798. Another primer extension method is allele-specific PCR (Ruano et al., Nucl. Acids Res. 17:8392, 1989; Ruano et al., Nucl. Acids Res. 19, 6877-6882, 1991; WO 93/22456; Turki et al., J Clin. Invest. 95:1635-1641, 1995). In addition, multiple polymorphic sites may be investigated by simultaneously amplifying multiple regions of the nucleic acid using sets of allele-specific primers as described in Wallace et al. (WO89/10414).

Another preferred method for genotyping the KRAS variant is the use of two oligonucleotide probes in an OLA (see, e.g., U.S. Pat. No. 4,988,617). In this method, one probe hybridizes to a segment of a target nucleic acid with its 3′ most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

The following patents, patent applications, and published international patent applications, which are all hereby incorporated by reference, provide additional information pertaining to techniques for carrying out various types of OLA: U.S. Pat. Nos. 6,027,889, 6,268,148, 5494810, 5830711, and 6054564 describe OLA strategies for performing SNP detection; WO 97/31256 and WO 00/56927 describe OLA strategies for performing SNP detection using universal arrays, wherein a zipcode sequence can be introduced into one of the hybridization probes, and the resulting product, or amplified product, hybridized to a universal zip code array; U.S. application US01/17329 (and Ser. No. 09/584,905) describes OLA (or LDR) followed by PCR, wherein zipcodes are incorporated into OLA probes, and amplified PCR products are determined by electrophoretic or universal zipcode array readout; U.S. application 60/427,818, 60/445,636, and 60/445,494 describe SNPlex methods and software for multiplexed SNP detection using OLA followed by PCR, wherein zipcodes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization—Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

Typically, the primer extension assay involves designing and annealing a primer to a template PCR amplicon upstream (5′) from a target SNP position. A mix of dideoxynucleotide triphosphates (ddNTPs) and/or deoxynucleotide triphosphates (dNTPs) are added to a reaction mixture containing template (e.g., a SNP-containing nucleic acid molecule which has typically been amplified, such as by PCR), primer, and DNA polymerase. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target SNP site) or two or more nucleotides removed from the SNP position. If the primer is several nucleotides removed from the target SNP position, the only limitation is that the template sequence between the 3′ end of the primer and the SNP position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target SNP position. In this instance, primers are designed to bind one nucleotide upstream from the SNP position (i.e., the nucleotide at the 3′ end of the primer hybridizes to the nucleotide that is immediately adjacent to the target SNP site on the 5′ side of the target SNP site). Extension by only one nucleotide is preferable, as it minimizes the overall mass of the extended primer, thereby increasing the resolution of mass differences between alternative SNP nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer.

The extended primers can then be purified and analyzed by MALDI-TOF mass spectrometry to determine the identity of the nucleotide present at the target SNP position. In one method of analysis, the products from the primer extension reaction are combined with light absorbing crystals that form a matrix. The matrix is then hit with an energy source such as a laser to ionize and desorb the nucleic acid molecules into the gas-phase. The ionized molecules are then ejected into a flight tube and accelerated down the tube towards a detector. The time between the ionization event, such as a laser pulse, and collision of the molecule with the detector is the time of flight of that molecule. The time of flight is precisely correlated with the mass-to-charge ratio (m/z) of the ionized molecule. Ions with smaller m/z travel down the tube faster than ions with larger m/z and therefore the lighter ions reach the detector before the heavier ions. The time-of-flight is then converted into a corresponding, and highly precise, m/z. In this manner, SNPs can be identified based on the slight differences in mass, and the corresponding time of flight differences, inherent in nucleic acid molecules having different nucleotides at a single base position. For further information regarding the use of primer extension assays in conjunction with MALDI-TOF mass spectrometry for SNP genotyping, see, e.g., Wise et al., “A standard protocol for single nucleotide primer extension in the human genome using matrix-assisted laser desorption/ionization time-of-flight mass spectrometry”, Rapid Commun Mass Spectrom. 2003; 17(10:1195-202.

The following references provide further information describing mass spectrometry-based methods for SNP genotyping: Bocker, “SNP and mutation discovery using base-specific cleavage and MALDI-TOF mass spectrometry”, Bioinformatics. 2003 July; 19 Suppl 1:144-153; Storm et al., “MALDI-TOF mass spectrometry-based SNP genotyping”, Methods Mol. Biol. 2003; 212:241-62; Jurinke et al., “The use of MassARRAY technology for high throughput genotyping”, Adv Biochem Eng Biotechnol. 2002; 77:57-74; and Jurinke et al., “Automated genotyping using the DNA MassArray technology”, Methods Mol. Biol. 2002; 187:179-92.

SNPs can also be scored by direct DNA sequencing. A variety of automated sequencing procedures can be utilized ((1995) Biotechniques 19:448), including sequencing by mass spectrometry (see, e.g., PCT International Publication No. WO94/16101; Cohen et al., Adv. Chromatogr. 36:127-162 (1996); and Griffin et al., Appl. Biochem. Biotechnol. 38:147-159 (1993)). The nucleic acid sequences of the present disclosure enable one of ordinary skill in the art to readily design sequencing primers for such automated sequencing procedures. Commercial instrumentation, such as the Applied Biosystems 377, 3100, 3700, 3730, and 3730.times.1 DNA Analyzers (Foster City, Calif.), is commonly used in the art for automated sequencing.

Other methods that can be used to genotype the KRAS variant include single-strand conformational polymorphism (SSCP), and denaturing gradient gel electrophoresis (DGGE) (Myers et al., Nature 313:495 (1985)). SSCP identifies base differences by alteration in electrophoretic migration of single stranded PCR products, as described in Orita et al., Proc. Nat. Acad. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel (Erlich, ed., PCR Technology, Principles and Applications for DNA Amplification, W. H. Freeman and Co, New York, 1992, Chapter 7).

Sequence-specific ribozymes (U.S. Pat. No. 5,498,531) can also be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis

SNP genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

### EXAMPLES

### Example 1

### The KRAS Variant in Triple-Negative Breast Cancer (TNBC)

### Study Populations

In this case-control study and genetic analysis, data were assessed from four cohorts (Table 1). To assess frequency distributions of the KRAS-variant genotype, individuals from the Yale Breast Cancer Study (study group 1) were assessed. Individuals from the Yale Breast Cancer Study (study group 1) were enrolled in a breast cancer case-control study in Connecticut, USA; which was approved by the Yale institutional review board (Hoffman A, et al. Cancer Res 2009; 69: 5970-77). Briefly, patients were aged 30-80 years and had incident, histologically confirmed breast cancer and no history of cancer (other than non-melanoma skin cancer). ER and PR statuses were established for all cases but HER2 statuses were not known and not obtainable. Controls were recruited either from Yale-New Haven Hospital (New Haven, Conn., USA) or Tolland County, Conn., USA. Controls from the Yale-New Haven Hospital underwent breast-related surgery for histologically confirmed benign breast diseases. Controls from Tolland County were identified either through random-digit dialing (for individuals aged <65 years) or through the Health Care Finance Administration files (=65 years). Informed consent and data for family histories of cancer, reproductive history, demographic factors, and blood sample were obtained from all participants. 415 cases and 457 controls had DNA samples available for this study, which were obtained between 1990 and 1999.

TABLE 1
Study Groups

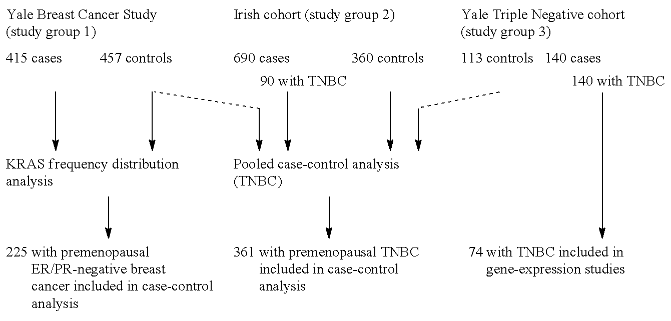

TNBC = triple negative breast cancer, ER = estrogen receptor, and PR = progesterone receptor.

To define the association of the KRAS variant with receptor status and breast cancer subtype, a cohort of 690 Irish women diagnosed with breast cancer with complete receptor status and subtype classification was assessed. Patients from this cohort (study group 2) had histologically confirmed breast cancer and were recruited from the west of Ireland after appropriate ethical approval from the Galway University Hospital (Galway, Ireland) ethics committee. Informed consent and a detailed family history of breast cancer or ovarian cancer, and a blood sample were obtained from all cases. 710 cases of breast cancer of all stages and histological types, apart from preinvasive carcinomas. ER, PR, and HER2 statuses were established for all samples by use of standard histopathological analysis and immunohistochemistry, and confirmed by fluorescence in-situ hybridization for HER2 positivity. These samples were classified as luminal A, luminal B, HER2, or triple-negative breast cancer by receptor status (Table 2). 690 of the 710 patients had complete information and were assessed in this study. The 360 controls in this cohort were healthy women from the same geographical area, and were mainly older than 60 years, with no self-reported personal history of any cancer and no family history of breast cancer or ovarian cancer. Cases and controls were mainly recruited from July, 2006, to July, 2010.

TABLE 2
Receptor Status of Subtypes.

| Breast Cancer Subtypes | ER | PR | Her-2 |
|---|---|---|---|
| Luminal A | +/− | +/− | − |
| Luminal B | +/− | +/− | + |
| Her-2+ | − | − | + |
| Triple Negative | − | − | − |

To establish whether the KRAS variant predicted an increased risk of development of triple-negative breast cancer, a pooled analysis was performed of a cohort of patients with triple-negative breast cancer and controls from Yale (study group 3), patients with triple-negative breast cancer and controls from study group 2, and controls from study group 1. Patients in study group 3 were receiving treatment either at Yale-New Haven Hospital or at the Bridgeport Hospital (Bridgeport, Conn., USA). After approval by the Yale Human Investigation Committee, tissue or saliva specimens were obtained from 156 patients. Complete data were available for 140 patients who were diagnosed in 1990-2007 and were included in this study. 130 cases of triple-negative breast cancer had samples of tumor available before any treatment for gene and miRNA-expression analysis, 78 of whom were also genotyped for the KRAS variant. 113 controls in this cohort were healthy women who presented to the Yale-New Haven Hospital and who had no personal history of cancer apart from nonmelanoma skin cancer and were recruited between 2000 and 2007. Clinical information, age, ethnic origin, and family history were obtained for all cases and controls. Table 3 summarizes basic information for these three cohorts.

TABLE 3
Description of the three separate breast cancer
case-control cohorts utilized in the study.

| | | Available | |
|---|---|---|---|
| Cohort | | receptor | Age |
| Name | Ascertainment criteria | status | (years) |
| Yale Case- | | | |
| Control | | | |
| Cases | Histologically confirmed BC | ER and | 30-80 |
| | cases, no prior history of | PR | |
| | cancer (except non-melanoma | | |
| | skin cancer) from CT, USA | | |
| Controls | Cancer free healthy subjects | | 35-85 |
| | or subjects who underwent | | |
| | surgery for histologically con- | | |
| | firmed benign breast disease. | | |
| Irish | | | |
| Cohort | | | |
| Cases | Histologically confirmed BC | ER, PR, | 30-80 |
| | cases from west of Ireland | and HER2 | |
| Controls | Healthy females, no self-reported | | >60 |
| | personal history of any cancer, | | |
| | no family history of breast or | | |
| | ovarian cancer | | |
| Yale TN | | | |
| cohort | | | |
| TNBC | Patients being treated eithe at | ER, PR, | 30-85 |
| cases | YNHH in New Haven or at | and HER2 | |
| | Bridgeport Hospita in | | |
| | Bridgeport, CT. | | |
| Controls | Subjects with no prior history of | | 30-80 |
| | cancer (except non-melanoma skin | | |
| | cancer) | | |

To assess association of the KRAS variant with BRCA mutations in ER-negative tumors, BRCA1-mutation carriers with breast cancer and known KRAS-variant status from our previous study of the Rotterdam population were analyzed. The Rotterdam population has been described (Hollestelle A, et al. Breast Cancer Res Treat 2010; published online July 30. DOI:10.1007/s10549-010-1080-z) but, briefly, this population included Dutch patients with breast cancer and documented BRCA1 mutations who were identified by investigators at the Erasmus University through the Rotterdam Family Clinic (Rotterdam, Netherlands).

### Procedures

KRAS-variant genotyping assays: DNA from all samples was genotyped for the KRAS variant using a custom Taqman SNP genotyping assay. Samples heterozygous or homozygous for the variant G allele were considered positive for the KRAS-variant (Chin L, et al. Cancer Res 2008; 68: 8535-40).

Gene expression analysis: Genome-wide mRNA expression was measured in 78 patients from the Yale triple-negative cohort who were also tested for the KRAS variant. Total RNA was isolated from tissue specimen with the RecoverAll total nucleic acid isolation kit (Applied Biosystems) and hybridized to the whole genome DASL assay (HumanRef-8 version 3.0, Illumina, San Diego, Calif., USA). Data preprocessing and statistical analysis were done with the lumi package in Bioconductor/R software. Gene-expression data from three whole-genome DASL runs were combined and processed together. Samples with less than 30% detectable probes and probes that were detectable in less than 10% of the samples were discarded before quantile-normalization. 74 samples and 18345 probes remained after filtering.

MicroRNA analysis: MicroRNA arrays were performed using the Multiplex RT and TaqMan low density array human miRNA panel-real-time PCR system (Applied Biosystems) as per the manufacturer's protocol (miRNA profiling, publicly available at www.appliedbiosystems.com/absite/us/en/home/applications-technologies/real-time-per/mirna-profiling.html (accessed Jan. 1, 2008). Expression levels of miRNAs of interest were examined.

### Statistical Analysis

Genotype distributions of all cases and controls were tested for Hardy-Weinberg equilibrium and were found to be in equilibrium. Unconditional logistic regression was performed to estimate the relative risk associated with every genotype. Controls were adjusted for age (continuous) and ethnic origin (white, black, Hispanic, or other). The population was stratified by menopausal status (estimated by age ≦51 years or >51 years), and separate risk estimates were obtained by ER and PR statuses with multinomial logistic regression with a three-level outcome variable coded as 0 for controls, 1 for cases with ER-positive and/or PR-positive tumors, and 2 for ER/PR-negative tumors. Wald χ² tests for interaction were performed, comparing the parameter estimates obtained for every genotype in cases of ER-positive and/or PR-positive disease compared with ER/PR-negative disease.

Patients in study group 2 were stratified according to the subtype of breast cancer and a χ² test was performed using the GraphPad Prism4 software to calculate the p values, odds ratios (Ors), and 95% confidence interval (CI). The dominant model was used for all genetic association analysis due to the low frequency of KRAS variant.

Categorical variables (e.g., ethnic origin, stage, and study site) were compared between study groups with a χ² test or two-sided Fisher's exact test, and continuous variables (e.g., age) with at test. ORs and a 95% CI were calculated for the KRAS variant in controls and cases of triple-negative breast cancer with an unconditional logistic regression model with a binary outcome variable. Multivariate logistic regression analyses with a binary outcome variable coded as controls and cases included variables such as KRAS-variant status, age, ethnic origin, and study site. The population was also stratified by age group, and separate logistic regression analyses were done for patients aged 51 years or younger (premenopausal group) or older than 51 years (postmenopausal group). Statistical analyses were done with SAS version 9.1.3.

Pathway activation was measured as correspondence with previously published expression signatures and axes derived from principal component analysis of the expression set. Principal component analysis was used to separate biological from technical sources of information in the gene-expression dataset. Every component was characterized by correspondence to RNA quality, the structure of a batch effect, and biological annotations of the contributing probes (i.e., probes with expression profiles that have high absolute projection values for the specified component). Signatures of gene expression are provided as lists of genes and their changes in expression in a specific condition. Such signatures are especially valuable for noisy data because they require coordinated differential expression of multiple probes, typically in the order of 100. Because mRNA was extracted from formalin-fixed, paraffin-embedded (FFPE) blocks that were up to 20 years old, analysis of the data set with a signature approach was justified (Kibriya M, et al. BMC Genomics 2010; 11: 622). S signature scores were calculated as Pearson correlation between the respective signature vector of gene contributions and a sample's expression profile for these genes. Association of the KRAS variant with the outcomes described by the respective signature was analyzed by a paired Kolmogorov-Smirnov test between signatures scores of KRAS variant and wild type samples. Differential gene expression was assessed with a linear model, taking into account technical batch artifacts as an offset. Model fitting and empirical Bayesian error moderation of the fold changes were performed with the LIMMA package for R (Smyth G K. Limma: linear models for microarray data. In: Gentleman R, et al, eds. Bioinformatics and computational biology solutions using R and bioconductor. New York, USA: Springer, 2005: 397-420).

MiRNA expression was analyzed in 8 batches of 46 miRNAs and 2 endogenous controls each. MicroRNA expression was normalized using the geometric mean over all expressed samples: A miRNA was judged have been expressed if threshold fluorescence was detected after less than 35 cycles (ct<35) and the geometric mean cycle number of all expressed miRNAs was subtracted. miRNAs that were not expressed in more than two thirds of all samples were removed, followed by scale-normalization over all remaining threshold cycle (Cₜ) values.

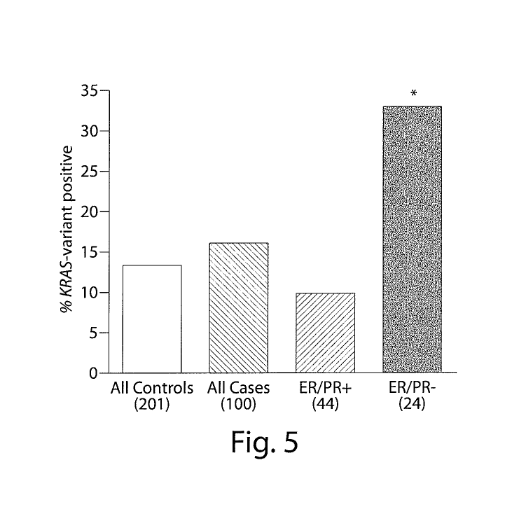

Frequency distributions of the KRAS-variant genotype did not differ between cases and controls who were genotyped from study group 1 (Table 1 and Table 4). However, the KRAS variant was significantly associated with breast cancer in premenopausal patients with ER/PR negative tumors (Table 4). This association was not observed for postmenopausal women. Eight (33%) of 24 premenopausal women with ER/PR-negative cancer had the KRAS variant, compared with 27 (13%) of 201 controls and four (9%) of 44 premenopausal women with cancer that was positive for ER and/or PR (FIG. 5). Thus, the KRAS variant might be a genetic marker of increased risk of development of receptor-negative breast cancer for premenopausal women.

TABLE 4
Association of the KRAS-variant with ER/PR positive versus ER/PR negative breast cancer.

| | | All | | ER and/or PR positive | | ER/PR negative | | |
|---|---|---|---|---|---|---|---|---|
| | Controls | Cases | Odds ratio (95% CI)* | Cases | Odds ratio (95% CI)* | Cases | Odds ratio (95% CI)* | P [symbol] |
| All ages | | | | | | | | |
| Non-variant (T/T) | 391 | 347 | Reference | 145 | Reference | 62 | Reference | — |
| Variant (T/G or G/G) | 79 | 68 | 0.95 (0.67-1.36) | 28 | 0.93 (0.58-1.49) | 18 | 1.59 (0.88-2.86) | 0.118 |
| Premenopausal | | | | | | | | |
| Non-variant (T/T) | 174 | 84 | Reference | 40 | Reference | 16 | Reference | — |
| Variant (T/G or G/G) | 27 | 16 | 1.64 (0.79-3.43) | 4 | 0.87 (0.28-2.75) | 8 | 4.78 (1.71-13.38) | 0.015 |
| Postmenopausal | | | | | | | | |
| Non-variant (T/T) | 217 | 263 | Reference | 105 | Reference | 46 | Reference | — |
| Variant (T/G or G/G) | 52 | 52 | 0.77 (0.51-1.16) | 24 | 0.90 (0.53-1.53) | 10 | 0.90 (0.43-1.90) | 0.991 |

Data are number or odds ratio (95% CI), unless otherwise stated.
ER = oestrogen receptor.
PR = progesterone receptor.
*Age ethnic origin, and menopausal status were adjusted in monomial unconditional logistic regression, G/G phenotype occurs in less than 5% of cases and controls and was combined with the G/T phenotype. Minor allele frequency (controls) 0.087, p for Hardy-Weinberg equilibrium 0.783. [symbol]
indicates data missing or illegible when filed

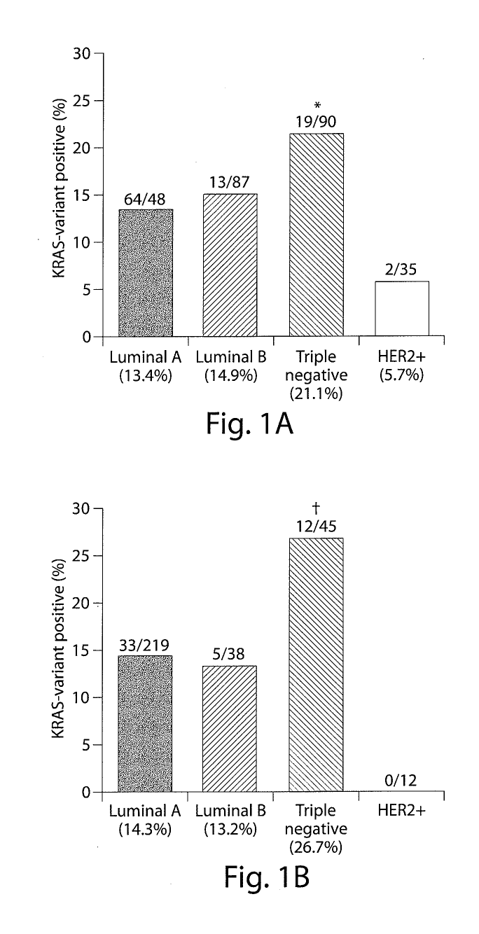

In study group 2, 478 women had luminal A breast cancer, 87 had luminal B breast cancer, 90 had triple-negative breast cancer, and 35 had HER2-positive breast cancer. 98 (14%) of 690 breast-cancer cases from this cohort had the KRAS variant, but prevalence varied between the breast cancer subtypes: The KRAS variant was statistically significantly enriched in women with triple-negative breast cancer (19 [21%] of 90 cases) compared with 64 (13%) of 478 for luminal A, 13 (15%) of 87 for luminal B, and two (6%) of 35 for HER2-positive subgroups (p=0.044; FIG. 1). This association with triple-negative breast cancer was also noted in women younger than 51 years (p=0.033, FIG. 1).

By comparison of cases of triple-negative breast cancer from groups 2 and 3 and controls across all three cohorts (n=1160), a statistically significant difference was found between cases or between controls for the prevalence of the KRAS variant (Table 5). There were more non-white women in the controls from study groups 1 and 3 than there were in the study group 2, which allowed assessment of the association of the KRAS variant in non-white women with triple-negative breast cancer in the multivariate analysis. After controlling for age, ethnic origin, and study site, the KRAS variant did not predict an increased risk of development of triple-negative breast cancer for all women in multivariate analysis (Table 6 and Table 7). However, the KRAS variant was associated with a statistically significant increased risk of development of triple-negative breast cancer in the 361 premenopausal women in this pooled group in multivariate analysis (Table 6, Table 8, and Table 9).

TABLE 5
Demographic variables for TNBC cases (A) and controls
(B) from the Irish cohort versus Yale cohort using Chi-
square test for categorical variable such as ethnicity
and t-test for the continuous variable (i.e. age.)
A. TNBC cases

| | Variable | | |
|---|---|---|---|
| | Ireland | Yale | P |
| | (n = 90) | (n = 140) | value |
| Age | 52.09 (10.66) | 53.2 (13.03) | 0.4995 |
| Ethnicity | | | <0.0001 |
| Caucasian (n = 166) | 90 (100.00) | 76 (54.29) | |
| African | 0 (0.00) | 50 (35.71) | |
| American (n = 50) | | | |
| Hispanic (n = 11) | 0 (0.00) | 11 (7.86) | |
| Asian American (n = 3) | 0 (0.00) | 3 (2.14) | |
| KRAS status | | | 0.3863 |
| Wild type (n = 188) | 71 (78.89) | 117 (83.57) | |
| Variant (n = 42) | 19 (21.11) | 23 (16.43) | |

B. Controls

| | Variable | | |
|---|---|---|---|
| | Ireland | Yale | P |
| | (n = 360) | (n = 570) | value |
| Age | 70.78 (6.78) | 55.14 (11.02) | <0.0001 |
| Ethnicity | | | <0.0001 |
| Caucasian (n = 881) | 360 (100.00) | 521 (91.40) | |
| African | 0 (0.00) | 44 (7.72) | |
| American (n = 44) | | | |
| Hispanic (n = 5) | 0 (0.00) | 5 (0.88) | |
| KRAS status | | | 0.9271 |
| Wild type (n = 780) | 303 (84.17) | 477 (83.68) | |
| Variant (n = 150) | 57 (15.83) | 93 (16.32) | |

TABLE 6
Association of the KRAS-variant in 230 patients with
triple negative breast cancer compared with 930 controls
from pooled analysis of study groups 1-3.

| | Odds ratio (95% CI) | p value |
|---|---|---|
| All ages | | |
| Univariate analysis | | |
| KRAS variant | 1.162 (0.797-1.694) | 0.4363 |
| Multivariate analysis | | |
| KRAS variant | 1.352 (0.901-2.028) | 0.1455 |
| Age | 0.913 (0.942-0.967) | <0.0001 |
| Ethnic origin | 2.536 (2.784-5.999) | <0.0001 |
| Premenopausal women | | |
| Univariate analysis | | |
| KRAS variant | 1.879 (1.067-3.310) | 0.029 |
| Multivariate analysis | | |
| KRAS variant | 2.307 (1.261-4.219) | 0.0067 |
| Age | 0.913 (0.871-0.956) | 0.0001 |
| Ethnic origin | 2.536 (1.582-4.067) | 0.0003 |

Age, ethnic origin, menopausal status, and study site were adjusted in a logistic regression model. G/G phenotype occurs in less than 5% of cases and controls and was combined with the G/T phenotype.

TABLE 7
Demographic variables for TNBC cases and controls of all ages
using Chi-square test for a categorical variable such as ethnicity
and t-test for a continuous variable (e.g., age).

| | Variable | | |
|---|---|---|---|
| | Controls | Cases | |
| Demographics | (n = 930) | (n = 230) | P value |
| Age | 61.20 (12.26) | 52.77 (12.14) | <0.0001 |
| KRAS | | | 0.4293 |
| Wild type (n = 968) | 780 (83.87) | 188 (81.74) | |
| Variant (n = 192) | 150 (16.13) | 42 (18.26) | |
| Ethnicity | | | <0.0001 |
| Caucasian (n = 1047) | 881 (94.73) | 166 (72.17) | |
| African | 44 (4.73) | 50 (21.74) | |
| American (n = 94) | | | |
| Hispanic (n = 16) | 5 (0.54) | 11 (4.78) | |
| Asian (n = 3) | 0 (0.00) | 3 (1.30) | |

TABLE 8
Demographic variables for premenopausal TNBC cases and controls
of using Chi-square test for a categorical variable such as
ethnicity and t-test for a continuous variable (e.g., age).

| | Variable | | |
|---|---|---|---|
| | Controls | Cases | |
| Demographics | (n = 250) | (n = 111) | P value |
| Age | 45.37 (4.65) | 42.70 (5.80) | <0.0001 |
| KRAS | | | 0.0331 |
| Wild type (n = 300) | 215 (86.00) | 85 (76.58) | |
| Variant (n = 61) | 35 (14.00) | 26 (23.42) | |
| Ethnicity | | | <0.0001 |
| Caucasian (n = 297) | 219 (87.60) | 78 (70.27) | |
| African | 28 (11.20) | 24 (21.62) | |
| American (n = 52) | | | |
| Hispanic (n = 9) | 3 (1.20) | 6 (5.41) | |
| Asian (n = 3) | 0 (0.00) | 3 (2.70) | |

TABLE 9
Association of the KRAS variant with triple negative
breast cancer cases under 51 years of age versus
controls in the Irish and Yale cohorts.
Irish cohort*

| Variable | OR | 95% CI | P value |
|---|---|---|---|
| KRAS -variant | 1.933 | 0 942-3.966 | 0.0723 |

*Univariate analysis against all controls
Yale cohort

| Variable | OR | 95% CI | P value |
|---|---|---|---|
| KRAS -variant | 2.457 | 1.121-5.384 | 0.0248 |

Multivariate Analysis, controlled for race and age

Because BRCA1 coding sequence mutations are associated with risk of triple-negative breast cancer, and because the KRAS variant is enriched in BRCA1 mutation-carriers with breast cancer (Hollestelle A, et al. Breast Cancer Res Treat 2010; published online July 30. DOI:10.1007/s10549-010-1080-z), it was determined whether the association of the KRAS variant with premenopausal triple-negative breast cancer was due only to its association with carriers of BRCA1 mutation. Of 36 women with triple negative breast cancer from cohort 2 and 3 who were BRCA tested, 25 (69%) were BRCA negative and 11 (31%) were BRCA positive. Of these patients, eight (32%) BRCA-negative women had the KRAS variant compared with three (27%) women who were BRCA positive. These findings suggest that the KRAS variant is associated with an independent group of patients with triple-negative breast cancer without BRCA mutations.

An association was discovered between KRAS-variant status and ER or PR negative statuses in the Rotterdam population cohort (Hollestelle A, et al. Breast Cancer Res Treat 2010; published online July 30. DOI:10.1007/s10549-010-1080-z; Kibriya M, et al. BMC Genomics 2010; 11: 622), however, menopausal status was not considered in these studies. With respect to the results of the study described herein, an enrichment of the KRAS variant was not observed in 126 premenopausal BRCA1-mutation carriers who had ER/PR-negative breast cancer compared with 268 BRCA1-mutation-carriers from the Rotterdam cohort (21.8% vs 23.5%, p=0.95). Thus, association of the KRAS variant with premenopausal triple-negative breast cancer is independent of its association with BRCA1 mutations.

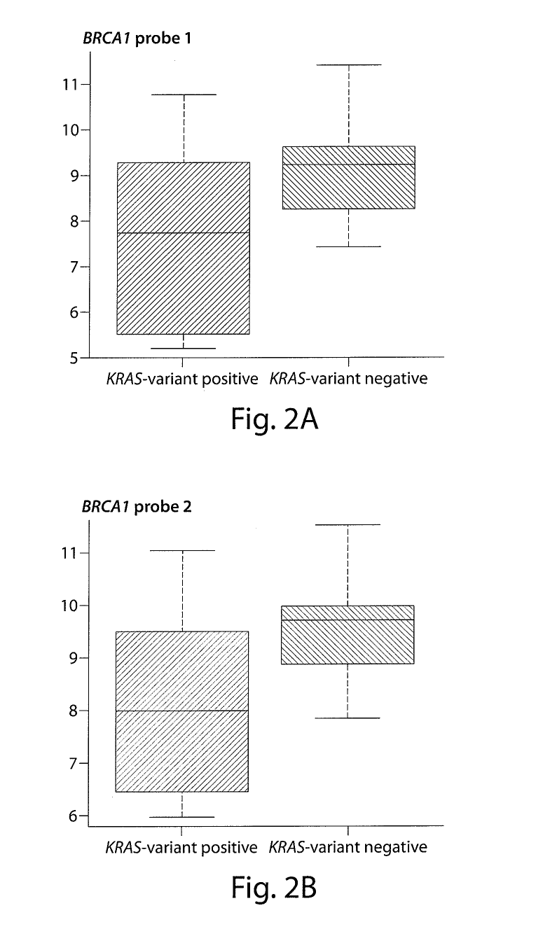

To further assess potential biological interaction between the KRAS variant and altered BRCA1 expression in triple-negative breast cancer, BRCA1 expression levels were determined in 74 triple-negative tumors from study group 3 (Table 1). Those patients with the KRAS variant demonstrated a statistically significant reduction of BRCA1 expression compared with KRAS variant-negative triple-negative tumors (p=0.06 for probe 1 [ILMN_{—}2311089] and p=0.01 for probe 2 [ILMN_{—}1738027], FIG. 2). Furthermore, the KRAS variant demonstrated a statistically significant association with a gene expression signature of decreased BRCA1 activity (p=0.04) (van't Veer L J, et al. Nature 2002; 415: 530-36). The data provided herein indicate that, although the KRAS variant is not restricted to patients with triple negative breast cancer with known BRCA1 mutations, a biological interaction between the KRAS variant, altered BRCA1 expression or functionality, and development of triple-negative breast cancer may exist.

Signaling pathways in triple-negative breast-cancer tumors that were KRAS-variant positive were compared with those that were KRAS-variant negative from patients in study group 3. Although analysis of KRAS mRNA did not vary by KRAS-variant status, the data are consistent with the other publications with respect to the effect of miRNA binding to the 3′-UTR of KRAS (Chin L, et al. Cancer Res 2008; 68: 8535-40; Johnson S M, et al. Cell 2005; 120: 635-47). An increase was found in both an NRAS mutation (Croonquist P A, et al. Blood 2003; 102: 2581-92) and a MAP-kinase activation signature (Creighton C J, et al. Cancer Res 2006; 66: 3903-11) (Table 10) in tumors with the KRAS variant. The data indicate that the KRAS variant alters gene expression of canonical RAS pathways. Moreover, the data provide the first in-vivo evidence that the KRAS variant leads to continued altered downstream gene expression in tumors with which it is associated.

TABLE 10
Association of the KRAS-variant with pathway signatures
in tumors of patients with triple negative breast
cancer and positive KRAS variant status.

| | Signature | Kolmogorov-Smirnov |
|---|---|---|
| | expression | p value |
| NRAS | Upregulated | 0.02 |
| BRCA mutant-like | Upregulated | 0.04 |
| Luminal progenitor | Upregulated | 0.04 |
| MAPK (Creighton) | Upregulated | 0.06 |
| PCA oestrogen | Downregulated | 0.04 |

Signature scores were computed as Pearson correlation between the signature vector of gene contributions and each sample's expression profile for these genes. The Kolmogorov-Smirnov test was used to analyse the association of the KRAS-variant with signature activation.

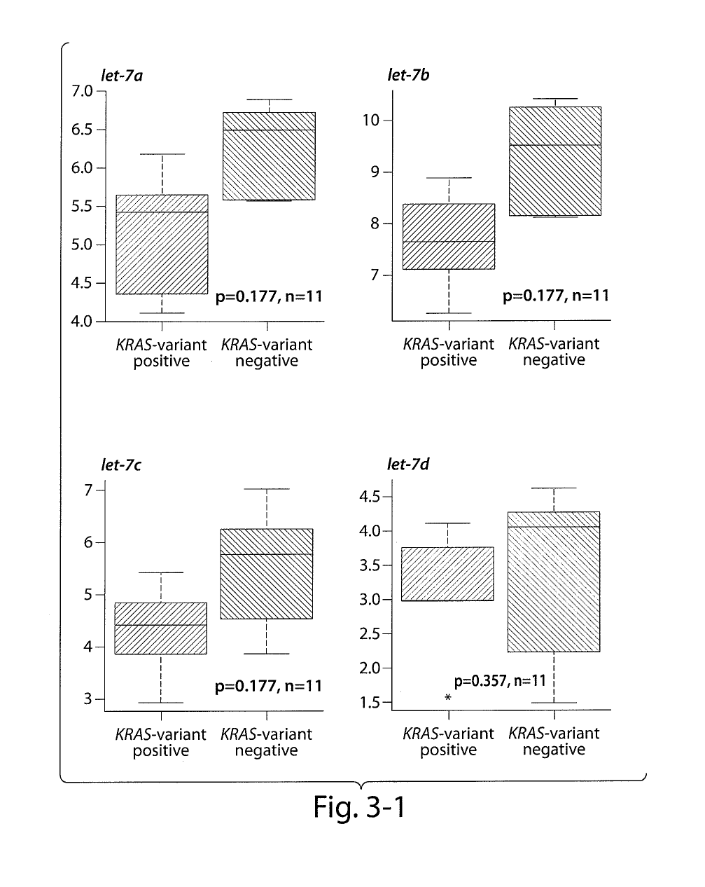

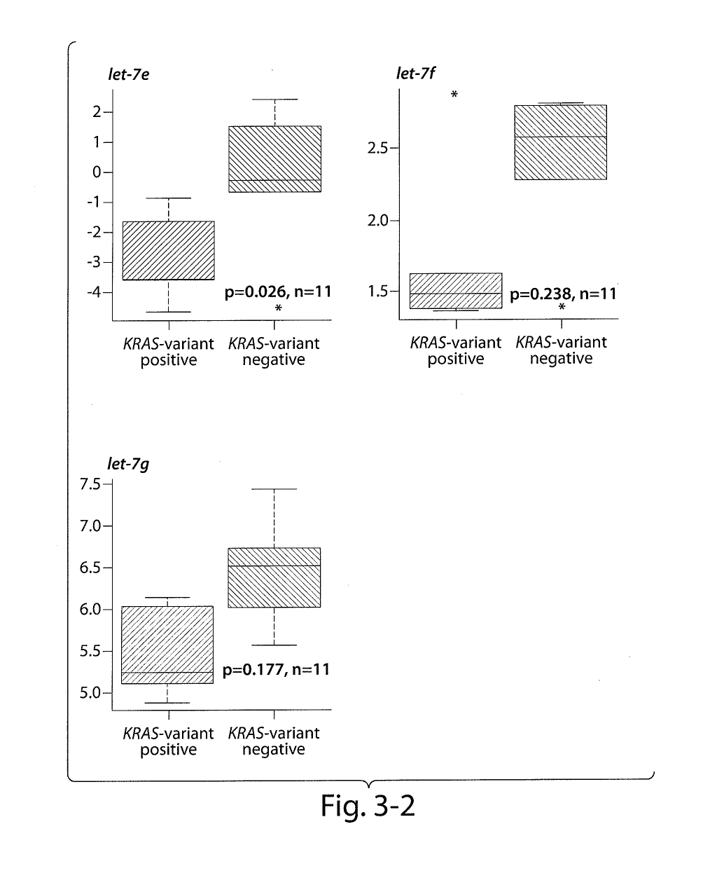

Because concentrations of let-7 miRNA are altered in lung tumors with the KRAS variant, let-7 concentrations were examined in triple-negative breast cancer tumors with the KRAS variant. The data demonstrated lower concentrations of all let-7 miRNA family members in KRAS-variant-associated tumors (FIG. 3).

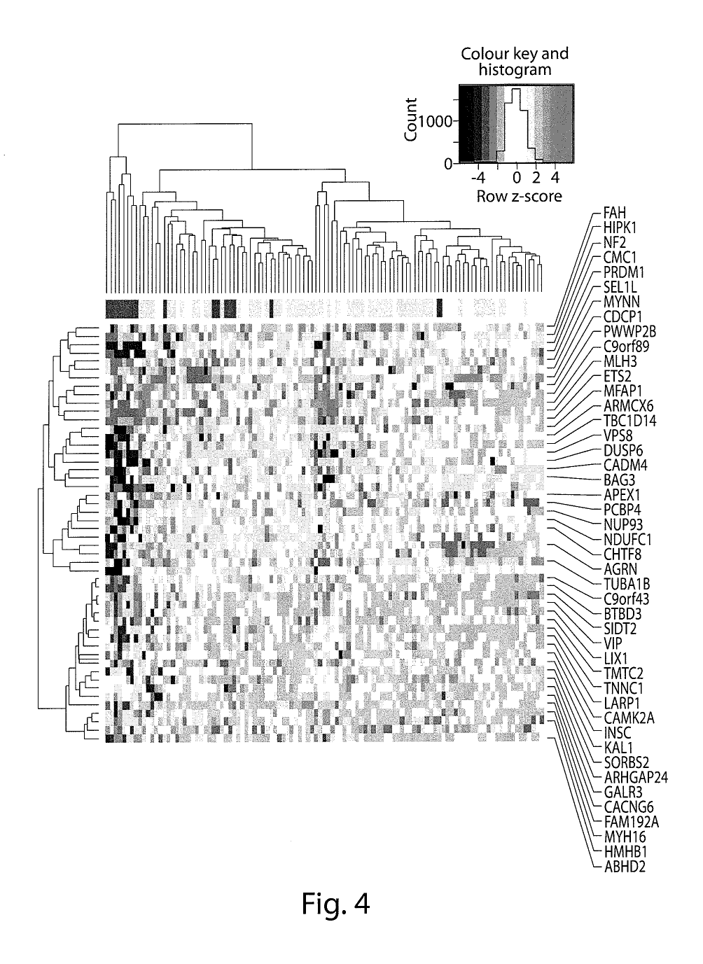

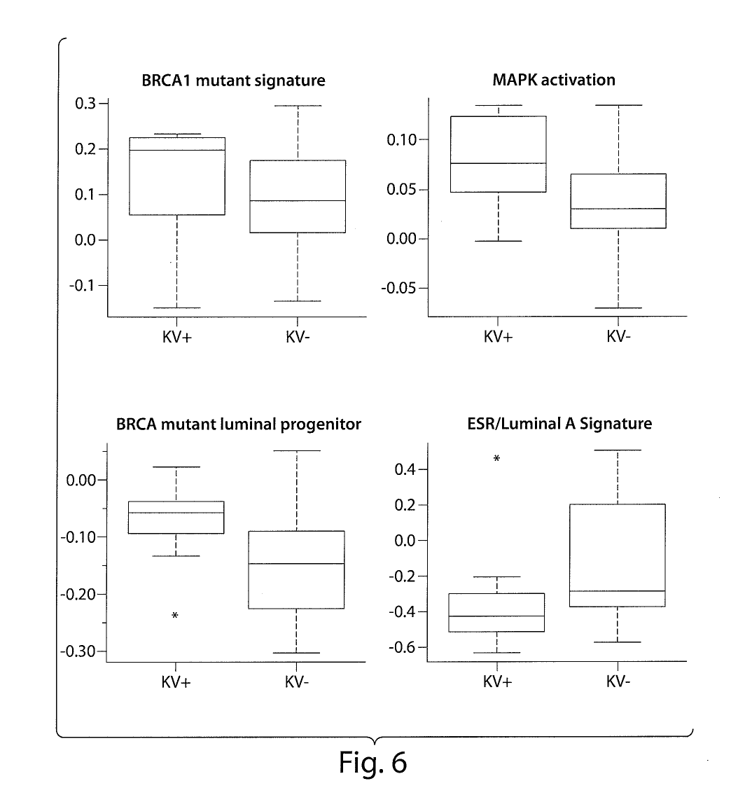

To establish how the KRAS variant integrates with known gene-expression signatures of triple-negative breast cancer, known signatures that are differentially expressed in such tumors were assessed. KRAS-variant tumors have several features of triple negative and basal-like tumor biology, including decreased estrogen signaling in a main component derived from the expression set (p=0.04). Furthermore, KRAS-variant tumors have a luminal progenitor signature (p=0.04), which is a candidate progenitor for basal-like breast cancer (Lim E, et al. Nat Med 2009; 15: 907-13) (Table 10 and FIG. 6). Within the luminal progenitor and the BRCA mutation-like signatures, markers of cell adhesion, tissue invasion, proliferation, and angiogenesis (such as α5 integrin, DUSP6, and aurora kinase B) were differentially regulated (Table 11). This discovery agrees with the slight enrichment by functional annotations that were observed in three of 41 genes for wound healing (p=0.02), three of 151 genes for glycan expression (p=0.05), and four of 148 genes for MEK activation (p=0.009) on the basis of the differentially expressed genes in a linear model comparing KRAS variant versus non-variant for the dataset (FIG. 4, Table 12 and Table 13).

TABLE 11
KRAS variant differently expressed genes within a luminal progenitor and
BRCA mutant signature by LIMMA analysis in triple negative breast cancer patients.

| Within luminal progenitor signature | | | | | Within the BRCA mutant like signature | | | | |
|---|---|---|---|---|---|---|---|---|---|
| nuID | gene | logFC | p | p adj | nuID | gene | logFC | p | p adj |
| WSdWOuc [symbol] PtRXftC [symbol] o | SCRB [symbol] 2 | 0.77024109 | 0.001 | 0.165 | reF_FIFUMuQSHsC [symbol] tr | DUSP6 | −1.5635473 | 0.000 | 0.0 [symbol] |
| [symbol] quxKuz6dLdfeA34 | PPP1RIB | 0.75990879 | 0.003 | 0.212 | Zr [symbol] [symbol] CDm [symbol] TP [symbol] . [symbol] 8l94 | T [symbol] | 0.80726644 | 0.001 | 0.11 |
| Ovtrl5a1erCxhhF3 [symbol] 8 | EPAS1 | −1.5277527 | 0.003 | 0.212 | 33oj158dC1fVkZDpoq | GPRC5C | −1.3698662 | 0.002 | 0.18 |
| Wo47wn13994KRR9_8_0 | WTDC3 | 0.76664801 | 0.003 | 0.212 | [symbol] Q56 [symbol] R4 | OGG1 | −0.0517652 | 0.004 | 0.24 |
| 314p_p [symbol] 45 [symbol] a12q.l0 | RAB24 | 0.6895776 | 0.009 | 0.338 | HIpqnaC6xyjCQuu [symbol] Z9O | GLRX2 | 0.47679698 | 0.005 | 0.24 |
| NRDIz22NlC [symbol] C9vk | AURKB | −1.5353.23 | 0.011 | 0.338 | NVt [symbol] QjfOa [symbol] Ekl | ZNF644 | 1.15014517 | 0.005 | 0.24 |
| [symbol] h6ezRdxBOW17BmE | AKR1C3 | −0.8935685 | 0.012 | 0.338 | fCkeqM.Snv1exVLU | UNC110 | 0.73173575 | 0.006 | 0.24 |
| raaukoERSnkqp41EBE | MATK | 0.49830612 | 0.012 | 0.338 | fkVEOXu10 [symbol] W5JeCE | VPRED3 | 0.92362289 | 0.025 | 0.47 |
| O5UqSufN4xwS5TTTro | TLR5 | −1.0574083 | 0.014 | 0.338 | rCOmicZud [symbol] CxxNhw | ETF8 | −2.05 [symbol] 8852 | 0.025 | 0.47 |
| 6Bvx9177TR4tJHrek | RAB24 | −0.8803527 | 0.014 | 0.338 | lAbvmX [symbol] WR [symbol] U4 | SORD | −0.8414537 | 0.026 | 0.47 |
| T [symbol] BLpWwNL [symbol] 713 [symbol] k | NCAID | −1.0265287 | 0.015 | 0.338 | u161oQDCFHTL [symbol] pQk | AFF1 | −0.7123633 | 0.028 | 0.47 |
| ropelrUnIS5fWDHf_o | UBE2C | −0.6930782 | 0.029 | 0.498 | c57 [symbol] Ul4hr15e36q_ug | RBM38 | 0.57005612 | 0.029 | 0.47 |
| IODd056ovTZT97vSeU | MCAID | −1.0912362 | 0.031 | 0.498 | WQp [symbol] g37 [symbol] WWDIKc | WT1 | 0.35637204 | 0.029 | 0.47 |
| cRUpD896.fUTE_ [symbol] o | PHL2 | −0.9577935 | 0.034 | 0.498 | xnIXICIfOUjef [symbol] kD | PPP1CB | 0.44366224 | 0.031 | 0.47 |
| [symbol] KthyQpF162oHYUCUEOI | CAMK2N1 | −0.090999 | 0.034 | 0.498 | [symbol] d [symbol] c9 [symbol] Ro3Q | ZFHK3 | 1.05484205 | 0.032 | 0.47 |
| fZrBCcM999_H1Lcv64 | PNMA2 | 0.60236064 | 0.035 | 0.498 | utBK [symbol] OuCk17gAb [symbol] o | P2RY10 | 0.58824966 | 0.033 | 0.47 |
| J [symbol] _kunvr9u [symbol] 3Q45QY | CAorf7 | 2.14470918 | 0.035 | 0.498 | HkD2UFRV [symbol] OK [symbol] 9T. [symbol] 4 | ANNAK | −0.5840241 | 0.034 | 0.47 |
| x2137 [symbol] zLOSOnju [symbol] IU | COL4A5 | −1.3394053 | 0.038 | 0.498 | KzhYQpF167cHVUCEC4 | CAMK2N1 | −0.098999 | 0.034 | 0.47 |
| No174RVAVBCgloguU | SLPl | 1.32821488 | 0.038 | 0.498 | 96N [symbol] Q [symbol] 6lO [symbol] ne. [symbol] | C14ORF116 | 0.36249749 | 0.035 | 0.47 |
| [symbol] HUgn O1N1GIFXE | CSN3 | 1.69487059 | 0.046 | 0.561 | z6 [symbol] _n546oo [symbol] As | RCN [symbol] 4 | 0.41693431 | 0.036 | 0.47 |
| [symbol] w71O3gvSVL [symbol] E561 | MAOA | −1.2134788 | 0.053 | 0.597 | Ko [symbol] 51gojafuvOv4oHo | NME1 | −0.469841 | 0.038 | 0.48 |
| Nkr_pVQ418gbOQOQ.56Q | MAF | 0.42034031 | 0.057 | 0.597 | 0.7 [symbol] UVUdoVU13CVU | C1ORF38 | 0.37871966 | 0.039 | 0.48 |
| O [symbol] uaqfpUroIEn_qF8 | F1K3F1 | 0.37430965 | 0.057 | 0.597 | WPw [symbol] 905 [symbol] _YOTsTu4 | NPTN | −0.2142517 | 0.045 | 0.54 |
| [symbol] QVgX [symbol] fFIRUofPw [symbol] | ITGA5 | −0.31653 | 0.058 | 0.597 | WLf_H_9Ek7k [symbol] RD4 | CA2 | 0.4792348 | 0.049 | 0.54 |
| [symbol] VwtXd_Agfe [symbol] d4Jy | C1QTNF1 | −1.0556673 | 0.061 | 0.598 | EqRaeX9VKg13 [symbol] fBQg | BP1 | 0.41133271 | 0.051 | 0.54 |
| [symbol] SdtSQUJA5M14O | HSPB6 | −1.1720388 | 0.064 | 0.607 | oc [symbol] 7ASQuq [symbol] Kd | DUSP1J | 0.37229160 | 0.056 | 0.54 |
| upHrr.S3ySrUQyeGno | NQOI | −0.8463655 | 0.067 | 0.607 | 6p1QpTgg [symbol] OpuxQsVY | STARDID | −0.640911 | 0.060 | 0.54 |
| IgCvB1 [symbol] 121_ [symbol] 5CC | CPE | −0.7809367 | 0.075 | 0.651 | lUfeKDox [symbol] ER_DO [symbol] jk | KLML2 | 0.40422699 | 0.062 | 0.54 |
| [symbol] RdfU74SUq [symbol] d_ [symbol] | C1QTNF1 | −0.9966795 | 0.077 | 0.651 | Npd2qV25 [symbol] e2WpxhZFO | NME2 | −1.282517 | 0.065 | 0.54 |
| 9 [symbol] GSG5K2 [symbol] kp [symbol] Ce9Fv8 | BMP4 | 0.49379944 | 0.081 | 0.651 | ZWm_wXSh [symbol] V_ [symbol] 5X92A | PUS1 | −0.6502441 | 0.065 | 0.54 |
| N SIAby_UdvHdNK [symbol] | KRT15 | 0.97895007 | 0.085 | 0.651 | uPHrr.53 [symbol] QreG [symbol] o | NQO1 | −0.463655 | 0.066 | 0.54 |
| [symbol] fprgRN4JRrv16dwp28 | NKD2 | −0.4423883 | 0.086 | 0.651 | [symbol] wg13OT4cTqooEnWpU | TNN11 | 0.37817669 | 0.068 | 0.54 |
| [symbol] 2nG [symbol] C1R [symbol] p1CQ | C19orf33 | −0.9280316 | 0.087 | 0.651 | IH79A5O [symbol] _n [symbol] O | MTC | −0.5960534 | 0.068 | 0.54 |
| rd [symbol] CJV5V3v [symbol] EvBgwo | MGR | −0.7374176 | 0.091 | 0.662 | rMDuo40OgDOoSu3604 | ZN [symbol] 44 | 0.30516255 | 0.070 | 0.54 |
| [symbol] MfdEnQfw [symbol] | SRPK2 | 0.36689011 | 0.097 | 0.680 | TgCvB [symbol] _4 [symbol] 5G [symbol] | CPE | −0.7809367 | 0.074 | 0.54 |
| [symbol] _IBI6ckOHc [symbol] sc.k_U | TMEM45A | 0.72481799 | 0.100 | 0.685 | opT1IU52E [symbol] hQpTj [symbol] Ao | DC1 | −0.8140602 | 0.076 | 0.54 |
| | | | | | HH576Rd [symbol] 7kDSQ [symbol] VFSA | INFP51 | −0.6589636 | 0.077 | 0.54 |
| | | | | | rXSTkN644 [symbol] SmqJSIA | DDX59 | 0.45766024 | 0.077 | 0.54 |
| | | | | | LxO1 [symbol] Pul [symbol] E | GPM6D | −0.9870874 | 0.080 | 0.55 |
| | | | | | lk8gpErppp [symbol] OJ [symbol] 9of | CRTM | 0.33291961 | 0.082 | 0.55 |
| | | | | | ZkcgDHZH10gv39_BG [symbol] | BMPR2 | 0.30205644 | 0.084 | 0.55 |
| | | | | | 90X [symbol] CE [symbol] OHv_ [symbol] j4SU | TCF7 | 0.83647349 | 0.091 | 0.55 |
| | | | | | ofv37Vg [symbol] Zdy57_Kro | FAM1298 | −0.2404329 | 0.096 | 0.55 |
| | | | | | [symbol] 5lggl [symbol] lop6 [symbol] | CRMDL3 | 0.90322229 | 0.096 | 0.55 |
| | | | | | Q [symbol] KpkXSrp [symbol] B2Fkfy [symbol] | NME2 | −0.5310338 | 0.097 | 0.55 |
| | | | | | [symbol] SOU [symbol] kg [symbol] | SSF1 | 0.36145747 | 0.098 | 0.55 |
| | | | | | rp [symbol] _rEr [symbol] nv [symbol] OX [symbol] | ITGB5 | −0.5851932 | 0.099 | 0.55 |

[symbol]

indicates data missing or illegible when filed

TABLE 12
Enrichment of selected literature-derived signatures with genes
identified to be KRAS variant differentially expressed by LIMMA
analysis in triple negative breast cancer patients.

| Signature | p. adj | p. raw | maxG | diff. Exp |
|---|---|---|---|---|
| ctnnb_bild2006 | 0.61600883 | 0.045721354 | 61 | 2 |
| glyc_potapenko09 | 0.61600883 | 0.052426283 | 151 | 3 |
| intrinsic_hu06 | 1 | 0.688155238 | 823 | 4 |
| mek_dry2010 | 0.43918195 | 0.009344297 | 148 | 4 |
| safb12_chiptargets | 0.7154859 | 0.091338625 | 429 | 5 |
| safb12_mrnatargets | 0.64952229 | 0.069098116 | 519 | 6 |
| wound.chang.down | 0.51678369 | 0.021990795 | 41 | 2 |
| wound.chang.up | 1 | 0.387976745 | 87 | 1 |

Abbreviations: p. adj: FDR-adjusted p-value, maxG: The number of genes in the signature represented on the Illumina microarray, diff. Exp: the number of genes in that signature found to be differentially expressed.

TABLE 13
List of 50 differentially expressed genes in triple
negative cancer patients who are KRAS variant positive,
as identified by LIMMA analysis.

| nuID | gene | logFC | p-value | p. adj |
|---|---|---|---|---|
| xu0rGiKPdOHt8XceDM | KAL1 | 1.161 | 3.27E−006 | 0.060 |
| 3l179x09bWKt66WJXg | ABHD2 | 1.197 | 3.08E−005 | 0.211 |
| 6p2cSR6PJ6AXXqIXE8 | CHTF8 | −1.762 | 5.41E−005 | 0.211 |
| 63x3kkE6KIv_7Li7qY | INSC | 0.952 | 5.80E−005 | 0.211 |
| 1E [symbol] 9 [symbol] LO6FVQLUiPlco | LIX1 | 0.862 | 7.67E−005 | 0.211 |
| lno3WL1Wlq14ompEqQ | BTBD3 | 0.892 | 9.30E−005 | 0.211 |
| uqeojrurej [symbol] 90ibdTk | SIDT2 | 0.704 | 9.45E−005 | 0.211 |
| uSgLvder_5UU65g14U | CAMK2A | 1.081 | 1.01E−004 | 0.211 |
| f95JXtx_pFVFF7ye68 | BAG3 | −2.012 | 1.21E−004 | 0.211 |
| rVUch7idfipSh83e9k | NA | −2.011 | 1.25E−004 | 0.211 |
| Z6n_xvX3TtHxtHH2U0 | SEL1L | 1.606 | 1.49E−004 | 0.211 |
| Bpeyv2kQv0mIgogB5c | ARMCX6 | −1.394 | 1.62E−004 | 0.211 |
| 3UCajp6zXipJBv0TCo | C9orf43 | 0.689 | 1.66E−004 | 0.211 |
| TYoWYd0HWC0JWR3jxc | NUP93 | −1.653 | 1.71E−004 | 0.211 |
| HkskAssd7rq8Nr_KSc | PRDM1 | 1.024 | 1.83E−004 | 0.211 |
| 0F8F [symbol] 38 [symbol] _7XU0efV4 | TUBA1B | −0.39 | 1.84E−004 | 0.211 |
| cKeVUq [symbol] eR61Xf9X0t8 | MLH3 | −1.911 | 1.99E−004 | 0.214 |
| Kn7e [symbol] HJ5ul_hq6eE3s | ETS2 | −1.661 | 2.27E−004 | 0.214 |
| reF_FiFUMuQSH [symbol] Os [symbol] Y | DUSP6 | −1.564 | 2.28E−004 | 0.214 |
| reltIN1nl3Ulf_SV78 | NA | −1.379 | 2.40E−004 | 0.214 |
| 3gq7osjnhfRUieXOVI | TNNC1 | 0.94 | 2.65E−004 | 0.214 |
| rmqB9Bd3eQp7hQjlQ1 | CDCP1 | −1.343 | 2.73E−004 | 0.214 |
| oWlPAI4KSFukWWXkAA | NA | −1.87 | 2.84E−004 | 0.214 |
| ir_h054gFRKAke1Kyg | MFAP1 | −1.08 | 3.04E−004 | 0.214 |
| xU75QpS3gNep0LjXXk | NA | 0.991 | 3.10E−004 | 0.214 |
| Qepf [symbol] oeledex5zWUg | C9orf89 | −1.841 | 3.12E−004 | 0.214 |
| KyB4s96IAUOtJH [symbol] _I | MYNN | 1.152 | 3.26E−004 | 0.214 |
| o017n3rGln2X0laUXE | GALP3 | 0.764 | 3.34E−004 | 0.214 |
| TfSd3QigkFQl [symbol] RbijY | NF2 | −1.571 | 3.39E−004 | 0.214 |
| ZHdUeJCbe61zrNBSzc | ARHGAP24 | 0.524 | 4.02E−004 | 0.232 |
| NLTejm0VUSrNidBmo | HIPK1 | −1.277 | 4.16E−004 | 0.232 |
| 6 [symbol] 8l5O [symbol] crfeULu [symbol] dL4 | TMTC2 | 0.728 | 4.19E−004 | 0.232 |
| ijpnYVZIUk3SE7YAI4 | CMC1 | −1.158 | 4.28E−004 | 0.232 |
| W5dWOuc9PtRXFIOHmo | SORBS2 | 0.77 | 4.45E−004 | 0.232 |
| 3ddSK3kR1H0Tlvh3RI | TBCID14 | −1.495 | 4.51E−004 | 0.232 |
| fadTvcHrug [symbol] BglTIXo | FAH | −1.265 | 4.55E−004 | 0.232 |
| 10 [symbol] qIh8QdN5J1q5o | CACNG6 | 0.695 | 5.00E−004 | 0.244 |
| 0Ch9ek_mecjpZuSdK8 | AGRN | −1.358 | 5.05E−004 | 0.244 |
| r5VigoIRK4SoglPlPU | LARP1 | 0.864 | 5.44E−004 | 0.247 |
| lh1833UT57TptL0Uh0 | VPS8 | −1.787 | 5.60E−004 | 0.247 |
| 6 [symbol] Tpd [symbol] EqRfed0r6uE | VIP | 0.655 | 5.60E−004 | 0.247 |
| QovQuig56f4ldxHur4 | NA | −1.636 | 5.77E−004 | 0.247 |
| QdJ_t1ZKfSABUPkbuo | PWWP2B | −1.276 | 5.79E−004 | 0.247 |
| Qp9WalX7nu6IXQ7pE4 | APEX1 | −0.558 | 6.01E−004 | 0.251 |
| 14AzSPkVV3VT16f_VU | CADM4 | −1.223 | 6.30E−004 | 0.252 |
| cpX0uY0C9xuYiWWDIA | NDUFC1 | −1.197 | 6.70E−004 | 0.252 |
| QuRcd6 [symbol] Qd4rSZIS4I | FAM192A | 1.657 | 6.74E−004 | 0.252 |
| rh2CVlv_u6OJKhA_eI | HMHB1 | 0.738 | 6.98E−004 | 0.252 |
| xyeqUp93VRReSP3558 | PCBP4 | −0.742 | 6.99E−004 | 0.252 |
| 6KL5K6C6EnZIKLlk7E | MYH16 | 0.77 | 7.04E−004 | 0.252 |

[symbol]

indicates data missing or illegible when filed

### Example 2

### Prevalence of the KRAS Variant in Various Cancer Cell Lines

### Materials and Methods

Genotyping. DNA from the NCI-60 cell line panel was obtained from the NCI's Developmental Therapeutics Program. Taqman genotyping was performed to determine the presence of the KRAS variant allele as described previously (Bussey K J, et al. Mol Cancer Ther 2006; 5:853-67). Cells were cultured under standard conditions (see, dtp.cancer.gov/branches/btb/ivclsp.html; Monks A, et al. J Natl Cancer Inst 1991; 83:757-66), for a maximum of 20 passages from frozen stock. DNA was isolated using the Qiagen QIAamp DNA blood maxi kit procedure (cat. 51192).

Statistical analyses. The KRAS variant allele data were coded numerically, with 1 representing the presence of KRAS variant allele and 0 representing the absence of the KRAS variant allele. This pattern was used as a “seed” in COMPARE analyses (Paull K D, et al. J Natl Cancer Inst 1989; 81:1088-9248) to probe the existing NCI-60 data sets in the NCI-DTP databases. Correlations included, for example, miRNA measurements and DNA methylation measurements. A positive correlation indicates, for example, that cell lines with the variant allele tend to have higher expression of the miRNA/mRNA or greater percentage DNA methylation. Conversely, negative correlations indicate that cell lines with the variant allele tend to have lower expression of a given miRNA/mRNA or lower percentage DNA methylation at the indicated gene. These data sets can be queried or downloaded at dtp.cancer.gov.

The presence of the KRAS variant is a genetic marker for prediction of risk and tumor biology as well as response to treatment in multiple cancers. The presence of the KRAS variant results in altered regulation by the KRAS 3′ UTR. This study elucidates the biological significance of the KRAS variant in cancer cells. The data provided herein elucidate exemplary molecular pathways that are affected by the presence of the KRAS variant. To simultaneously analyze a broad range of cancer types, the comprehensive NCI-60 panel of cancer cell lines (Blower P E, et al. Mol Cancer Ther 2007; 6:1483-91; Liu H, et al. Mol Cancer Ther 2010; 9:1080-91) was used. Various molecular parameters were studied to determine which molecular events correlate with the presence of the KRAS variant in these cancer cell lines (Kundu, S. T. et al. 2012 Jan. 15. Cell Cycle 11:2, 361-366).

Seven of 60 cell lines in the NCI-60 panel harbor the KRAS variant allele (Table 14). When the NCI-60 panel of cell lines were categorized based on the presence of either an acquired dominant mutation in the KRAS coding region (KRAS mutation) or the presence of the KRAS variant, it was determined that all seven cell lines that contained the KRAS variant were negative for the presence of KRAS-activating mutations. Similarly, the cell lines that carried a KRAS coding sequence mutation lacked the KRAS variant allele. Thus, the presence or occurrence of either a KRAS coding mutation or the KRAS variant allele is mutually exclusive in these cell lines. Furthermore, because this mutual exclusivity occurs in cell lines derived from a variety of cancer types, this mutual exclusivity is not specific to a particular tissue type. Rather, this mutual exclusivity is a common feature of these cancer cell lines regardless of origin. These results indicate that the occurrence of either of these two events alone (i.e., the occurrence of the KRAS variant or the occurrence of a KRAS coding mutation), is sufficient to affect tumorigenesis in these cancer types. These results also indicate that the level of KRAS activation caused by a canonical coding sequence mutation is functionally comparable to the elevated KRAS expression induced by the presence of the KRAS variant in the 3′ UTR. This mutual exclusivity of acquired KRAS coding mutations and the KRAS variant was also found in non-small cell lung cancer patients (Chin L J, et al. Cancer Res 2008; 68:8535-40) and in ovarian cancer patients (Ratner E, Cancer Res 2010; 70:6509-15), but not in colon cancer patients (Zhang W, et al. Ann Oncol 2011; 22:484-5; Zhang W, et al. Ann Oncol 2011; 22:104-9).

TABLE 14
Cell lines in the NCI-60 panel that harbor the KRAS variant allele
or a functional mutation in the coding sequence of KRAS.

| | | KRAS | KRAS |
|---|---|---|---|
| NCI-60 | Tissue | LC S6 | Mutation in |
| Cell Lines | Type | Variant | coding sequence |
| HCT-116 | Colon | 0 | 1 |
| NCI-H460 | NSCLC | 0 | 1 |
| A549/ATCC | NSCLC | 0 | 1 |
| OVCAR-5 | Ovarian | 0 | 1 |
| CCRF-CEM | Leukemia | 0 | 1 |
| HCT-15 | Colon | 0 | 1 |
| SN12C | Renal | 0 | 1 |
| NCI-H23 | NSCLC | 0 | 1 |
| SW-620 | Colon | 0 | 1 |
| MDA-MB-231/ATCC | Breast | 0 | 1 |
| RPMI-8226 | Leukemia | 0 | 1 |
| HOP-62 | NSCLC | 0 | 1 |
| MCF7 | Breast | 1 | 0 |
| 786-0 | Renal | 1 | 0 |
| IGROV1 | Ovarian | 1 | 0 |
| HCC-2998 | Colon | 1 | 0 |
| DU-145 | Prostate | 1 | 0 |
| EKVX | NSCLC | 1 | 0 |
| U251/SNB-19 | CNS | 1 | 0 |

To determine whether the cell lines having the KRAS variant allele show a conserved alteration in the expression of miRNAs, a statistical analysis was performed on the miRNA expression profiles that were generated from seven cell lines that contain the KRAS variant allele compared with the miRNA expression profiles of the remaining cell lines of the NCI-60 panel (Blower P E, et al. Mol Cancer Ther 2007; 6:1483-91; Gaur A, et al. Cancer Res 2007; 67:2456-68). The presence of the KRAS variant allele shows a statistically significant positive correlation with increased expression of miR-23, miR-27 and miR-210 (Table 15). MiR-23 and miR-27 are expressed from the same cluster and advance progression of angiogenesis and metastasis (Zhou Q, et al. Proc Natl Acad Sci USA 2011; 108:8287-92). For example, miR-23 and miR-27 are enriched in endothelial cells and highly vascularized tissue. Moreover, miR-23 and miR-27 elevate signaling pathways that are essential for angiogenesis by reducing the expression of Sprouty2 and Sema6A, which have anti-angiogenic functions. Blocking the function of either miR-23 or miR-27 leads to a decrease in capillary tube formation and migration in response to VEGF in vitro and reduced vascularization of postnatal retinas in vivo (Zhou Q, et al. Proc Natl Acad Sci USA 2011; 108:8287-92). The statistically significant positive correlation of the KRAS variant with increased expression of miR-23, miR-27 suggests that tumor cells having the KRAS variant allele are prone to growth and metastatic progression as a result of elevated levels of miR-23 and miR-27.

TABLE 15
MicroRNAs with statistically significant increased expression
in cell lines having the KRAS-variant allele.

| | By | Correla- | | Signif- | MicroRNAs |
|---|---|---|---|---|---|
| Kras SNP | Variable | tion | Count | icance | upregulated |
| Kras SNP | MT3049 | 0.51 | 59 | 4.27E−05 | microRNA |
| | | | | | hsa-miR-210N |
| Kras SNP | MT3048 | 0.49 | 59 | 8.31E−05 | microRNA |
| | | | | | hsa-miR-210 |
| Kras SNP | MT3076 | 0.47 | 59 | 0.000184 | microRNA |
| | | | | | hsa-miR-27b |
| Kras SNP | MT3066 | 0.45 | 59 | 0.000373 | microRNA |
| | | | | | hsa-miR-23b |
| Kras SNP | MT3077 | 0.44 | 59 | 0.030546 | microRNA |
| | | | | | hsa-miR-27bN |

The expression of miR-210 is statistically significantly correlated with the presence of the KRAS variant allele in cells. MiR-210 is a marker of chronic hypoxia. Moreover, miR-210 is associated with proliferation and metastasis of breast and melanoma tumors as well as poor prognosis. MiR-210 is a direct transcriptional target of HIF proteins. Elevated levels of miR-210 are required for tumor cell survival under conditions of hypoxia. MiR-210 directly regulates the expression of MNT, a MYC antagonist that is required for cell cycle arrest under hypoxia. Consequently, increased levels of miR-210 contribute to an override of cell cycle arrest under conditions of hypoxic stress in tumor cells. Because increased miR-210 expression is associated with the presence of the KRAS variant, tumor cells containing the KRAS variant survive and proliferate under hypoxic conditions.

The data provided herein demonstrate that the KRAS variant contributes to or initiates aberrant signaling pathways that control the expression of several miRNAs (including, for example, miR-23, miR-27 and miR-210). Perturbation of signaling pathways that regulate expression of miRNAs, such as miR-23, miR-27 and miR-210, results in the initiation, development, maintenance or augmentation of tumor proliferation and metastatic transformation.

Promoter methylation is one mechanism through which gene expression is silenced in many cancers because changes in the methylation status of gene promoters lead to reduction in gene expression. Specifically, DNA methylation is an epigenetic effect caused when CpG dinucleotides are methylated, often in the promoter region of genes. Because methylation blocks access to the promoter by molecules that mediate gene transcription, methylation of the promoter results in gene silencing. Different cancers show distinct methylation patterns, the result of which is alterations in gene expression signatures. Therefore, to determine whether there is an alteration in DNA methylation patterns in the tumor cell lines having the KRAS variant, the methylation status of these cell lines was compared with the non-KRAS variant lines in the NCI-60 panel (Ehrich M, et al. Proc Natl Acad Sci USA 2008; 105:4844-9). The presence of the KRAS variant allele shows a statistically significant positive correlation with increased methylation of the promoter of many genes, including, for example, Notch1, cyclin D3 and CNBP (also known as ZNF9) (Table 16).

TABLE 16
Genes with statistically significant promoter hyper-methylation in KRAS variant positive cell lines.

| Kras SNP | By Variable | Correlation | Count | Significance | Promoter Locus | Gene names |
|---|---|---|---|---|---|---|
| Kras SNP | MT9686 | 0.56 | 58 | 5.89E−06 | 156_ZNF9_001_CpG¹ | ZNF9 or CNBP |
| Kras SNP | MT9698 | 0.52 | 58 | 2.47E−05 | 156_ZNF9_001_CpG^{5.7} | ZNF9 or CNBP |
| Kras SNP | MT9695 | 0.52 | 58 | 0.00002697 | 156_ZNF9_001_CpG [symbol] | ZNF9 or CNBP |
| Kras SNP | MT9697 | 0.51 | 58 | 4.82E−05 | 156_ZNF9_001_CpG^{40.00} | ZNF9 or CNBP |
| Kras SNP | MT9694 | 0.49 | 58 | 0.00011305 | 156_ZNF9_001_CpG^{33.36} | ZNF9 or CNBP |
| Kras SNP | MT9689 | 0.43 | 58 | 0.00082522 | 156_ZNF9_001_CpG [symbol] | ZNF9 or CNBP |
| Kras SNP | MT5347 | 0.73 | 51 | 8.59E−10 | 014_CCND3_001_CpG [symbol] | Cyclin D3 |
| Kras SNP | MT5363 | 0.59 | 52 | 3.63E−06 | 014_CCND3_001_CpG^{6.8} | Cyclin D3 |
| Kras SNP | MT5346 | 0.55 | 35 | 0.00055557 | 014_CCND3_001_CpG [symbol] | Cyclin D3 |
| Kras SNP | MT5345 | 0.46 | 52 | 0.00058183 | 014_CCND3_001_CpG^{11.12} | Cyclin D3 |
| Kras SNP | MT7559 | 0.57 | 59 | 2.63E−06 | 091_NOTCH1_001_CpG⁰ | Notch 1 Ligand |
| Kras SNP | MT7549 | 0.53 | 59 | 0.00001846 | 091_NOTCH1_001_CpG [symbol] | Notch 1 Ligand |
| Kras SNP | MT7557 | 0.5 | 59 | 6.15E−05 | 091_NOTCH1_001_CpG^{22.2} [symbol] | Notch 1 Ligand |
| Kras SNP | MT7555 | 0.42 | 59 | 0.00079928 | 091_NOTCH1_001_CpG¹ [symbol] .20 | Notch 1 Ligand |

[symbol]

indicates data missing or illegible when filed

The role of Notch1 expression in cancers is diverse. In many tumors, Notch1 overexpression or activation drives cancer progression and metastasis. For example, Notch1 activation results in an increase in invasive and migratory characteristics of breast cancer cells. Alternatively, Notch1 overexpression in a MYC background induces adenomas in the mouse lung, leading to the formation of lung adenocarcinoma. Thus, the evidence indicates that Notch1 may function as an oncogene. In contrast, Notch1 may also function as a tumor suppressor. For example, inhibitory mutations in Notch1 have been identified in squamous cell carcinomas of the head and neck. Depletion of Notch1 in mouse skin keratinocytes results in enhanced tumorigenesis by chemical carcinogens or by oncogenic Ras. In cervical cancers positive for the human papillomavirus (HPV), Notch1 expression is decreased when compared with normal adjacent tissue. Overexpression of activated Notch1 in HPV-positive cervical cancers and neuroblastoma cells (Zage P E, et al. Pediatr Blood Cancer 2011) leads to growth inhibition. Considered together, the evidence show that Notch1 is dysregulated in many cancers and, in some instances, may function as a putative tumor suppressor. Because methylation of the Notch1 promoter is increased in KRAS variant-positive cancer cells, Notch1 expression may be reduced in cells carrying the KRAS variant allele, and, therefore, KRAS-variant cell lines may induce or maintain their tumorigenic potential by inhibiting the tumor suppressing effects of Notch1.

Cyclin D3 is the member of the cyclin family of cell cycle proteins that is required for the G₁/S transition of the cell cycle. In KRAS variant cell lines, promoter methylation of cyclin D3 is increased, which indicates repression of cyclin D3 transcription. Consequently, the evidence suggests two exemplary mechanisms in which either cyclin D3 is not required for the transformed phenotype of these cell lines or methylation of the cyclin D3 promotor blocks a transcriptional repressor of cyclin D3.

In contrast to Notch1 and cyclin D3, CNBP (cellular nucleic acid binding protein), also called ZNF9, is not associated with the development or progression of cancer. However, CNBP/ZNF9 is part of a complex that binds to the MYC promoter. When expression of MYC is dysregulated, MYC contributes to the development and progression of cancer. The mechanism by which the association of the KRAS variant with the methylation status of ZNF9 contributes to cancer progression in KRAS-variant cells is unclear.

Gene expression in the seven cell lines harboring the KRAS variant allele was compared with the profiles of the remaining cell lines in the NCI-60 panel to determine specific alterations in gene expression in these cell lines. As shown in Table 17, a gene whose elevated expression is statistically significantly correlated with the presence of the KRAS variant in the cell lines is glutathione S-transferase theta1 (GSTT1). The GSTT1 gene encodes a member of the glutathione S transferase family of human phase II detoxifying enzymes, which detoxifies complex metabolic byproducts, xenobiotics and drugs by conjugating a glutathione group to these compounds, thus making them more soluble and easily excreted out of the cell. The theta1 isoform has been implicated in several cancers. For example, increased expression of GSTT1 is statistically significantly correlated with aggressive bladder cancers. In other different tumors types, GSTT1 is nonfunctional or absent due to genetic polymorphism, thus leading to increased risk of carcinogenesis and poor prognosis as a result of an accumulation or increased accumulation of toxic metabolites.

TABLE 17
Genes with statistically significantly higher mRNA expression in KRAS positive cell lines.

| Kras SNP | By Variable | Correlation | Count | Significance | Genes upregulated |
|---|---|---|---|---|---|
| Kras SNP | GSTT1 | 0.62 | 59 | 1.72E−07 | Glutathione S-transferase theta1 |
| Kras SNP | SYT12 | 0.54 | 59 | 1.06E−05 | Synaptotagmin XII |
| Kras SNP | ITIH1 | 0.54 | 59 | 1.11E−05 | Inter-alpha (globulin) inhibitor H1 |
| Kras SNP | MAPK3 | 0.53 | 59 | 1.77E−05 | Mitogen-activated protein kinase 3 |
| Kras SNP | POLD1 | 0.52 | 59 | 2.26E−05 | Polymerase (DNA directed), delta1, catalytic |
| | | | | | subunit 125 kDa |
| Kras SNP | TNFAIP2 | 0.52 | 59 | 2.76E−05 | Tumor necrosis factor, alpha-induced protein 2 |
| Kras SNP | SELE | 0.51 | 59 | 3.15E−05 | Selectin E (endothelial adhesion molecule 1) |
| Kras SNP | GPLD1 | 0.51 | 59 | 4.02E−05 | Glycosylphosphatidylinositol specific phospholipase D1 |
| Kras SNP | HINT2 | 0.5 | 59 | 5.39E−05 | Histidine triad nucleotide binding protein 2 |
| Kras SNP | EFNA4 | 0.5 | 59 | 6.48E−65 | Ephrin-A4 |
| Kras SNP | MFAP1 | 0.49 | 59 | 7.77E−05 | Microfibrillar-associated protein 1 |
| Kras SNP | P4HB | 0.49 | 59 | 7.93E−05 | Procollagen-proline, 2-oxoglutarate 4-dioxygenase |
| | | | | | (proline 4-hydroxylase), beta polypeptide (protein |
| | | | | | disulfide isomerase-associated 1) |
| Kras SNP | SULT1E1 | 0.49 | 59 | 8.28E−05 | Sulfotransferase family 1E, estrogen-preferring, |
| | | | | | member 1 |
| Kras SNP | BARX1 | 0.49 | 59 | 9.16E−05 | BarH-like homeobox 1 |
| Kras SNP | RCE1 | 0.48 | 59 | 0.000129 | RCE1 homolog, prenyl protein peptidase (S. cerevisiae) |
| Kras SNP | KNG1 | 0.47 | 59 | 0.000147 | Kininogen 1 |
| Kras SNP | MAP2K4 | 0.47 | 59 | 0.000158 | Mitogen-activated protein kinase kinase 4 |
| Kras SNP | BCR | 0.47 | 59 | 0.000179 | Breakpoint cluster region |
| Kras SNP | HSC20 | 0.47 | 59 | 0.000198 | J-type co-chaperone HSC20 |
| Kras SNP | NR2E1 | 0.46 | 59 | 0.000288 | Nuclear receptor subfamily 2, group E, member 1 |
| Kras SNP | SRP14 | 0.45 | 59 | 0.000328 | Signal recognition particle 14 kDa (homologous Alu |
| | | | | | RNA binding protein) |
| Kras SNP | DDR1 | 0.45 | 59 | 0.000357 | Discoidin domain receptor family, member 1 |
| Kras SNP | DSG2 | 0.45 | 59 | 0.000384 | Desmoglein 2 |
| Kras SNP | CD151 | 0.45 | 59 | 0.000399 | CD151 antigen |
| Kras SNP | ACP2 | 0.44 | 59 | 0.00047 | Acid phosphatase 2, lysosomal |
| Kras SNP | GNAI1 | 0.44 | 59 | 0.000479 | Guanine nucleotide binding protein (G protein), |
| | | | | | alpha inhibiting activity polypeptide 1 |

Mitogen-activated protein kinase 3 (MAPK3) is a member of the MAP kinase family. Moreover, increased expression of mitogen-activated protein kinase 3 (MAPK3) is associated with the KRAS variant in cancer cells. MAPK3 transduces signals from extracellular cues to regulate intracellular processes, such as cell proliferation and differentiation. For example, increased expression of phosphorylated MAPK3 has been associated with aggressive colorectal tumors and metastatic meduloblastoma. Increased levels of KRAS in KRAS variant positive cancer cells are associated with an increase in MAPK3 mRNA. At least in part, increased MAPK3 expression induces an increase cellular proliferation and neoplastic progression in these cells. Similarly, the expression of another MAPK (MAP2K4) was increased in the KRAS-variant positive expression profile. Furthermore, KRAS and MAPK (MAPK3 and/or MAP2K4) may contribute to a synergistic interaction between KRAS and MAPK signaling in KRAS-variant cancer cells that induces or enhances cell proliferation and/or neoplastic progression.

Increased expression of Synaptotagmin-12 and increased expression of inter-a globulin inhibitor-H1 are positively correlated with the presence of the KRAS variant in cancer cell lines. Under normal conditions, synaptotagmins regulate calcium-dependent membrane trafficking during synaptic transmission. Although there is no evidence of an involvement of synaptotagmin-12 with cancer, overexpression of synaptotagmin-13, a family member of synaptotagmin-12, suppresses a transformed phenotype of cells derived from a rat liver tumor cell line. Overexpression of synaptotagmin-12 in KRAS variant-positive cancer cell lines indicates a deregulation of novel pathways involving syntaptotagmins in cancer cells. The inter-a (globulin) inhibitor H1 is the heavy chain of the plasma serine protease inhibitor. Functionally, the inter-a (globulin) inhibitor H1 is required for extracellular matrix stability. Though the role of the inter-a (globulin) inhibitor H1 in cancer remains unexplored, recent evidence indicates that the expression of inter-a (globulin) inhibitor H1 is either lost or repressed in various solid tumors, including, for example, tumors of the lung, colon and breast.

### Example 3

### The KRAS Variant and Patient Response to Treatment (Ovarian Cancer)

### Materials and Methods

Overall survival analysis cohorts. Complete clinical data and DNA from women diagnosed with EOC without known BRCA mutations were included from the following three institutions under individual International Review Board approvals. All protocols accrued patients prospectively at the time of their diagnosis to avoid selection bias. References indicate previous detailed descriptions of these patients: (1) Turin, Italy #1 (n=197) (Lu L, et al. (2007). Cancer Res 67:10117-10122), (2) Brescia, Italy #2 (n=59) (Ratner E, et al. (2010). Cancer Res 15: 6509-6515), and (3) the Yale New Haven Hospital (YNHH) (n=198). Yale patients were collected prospectively on two clinical trials at the Yale Medical School of newly diagnosed EOC patients diagnosed between 2000 and 2009 (Table 18).

TABLE 18
Clinicopathologic parameters for overall survival analysis.

| | Variable name | | |
|---|---|---|---|
| | Non-variant | KRAS-variant | p |
| | (n = 351) | (n = 103) | value |
| Age (standard | 60.44 (11.89) | 58.77 (11.59) | 0.2115 |
| deviation) | | | |
| Stage | | | 0.8627 |
| I | 52 (14.81) | 15 (14.56) | |
| II | 22 (6.27) | 6 (5.83) | |
| III | 193 (54.99) | 52 (50.49) | |
| IV | 80 (22.79) | 29 (28.16) | |
| Unknown | 4 (1.14) | 1 (0.97) | |
| Grade | | | 0.0507 |
| Well | 31 (8.83) | 14 (13.59) | |
| differentiated | | | |
| Moderately | 60 (17.09) | 8 (7.77) | |
| differentiated | | | |
| Poorly | 228 (64.96) | 74 (71.84) | |
| differentiated | | | |
| Unknown | 32 (9.12) | 7 (6.80) | |
| Histology | | | 0.1887 |
| Serous | 203 (57.83) | 52 (50.49) | |
| Endometrioid | 37 (10.54) | 16 (15.53) | |
| Undifferentiated | 7 (1.99) | 0 (0.00) | |
| Clear Cell | 21 (5.98) | 10 (9.71) | |
| Mucinous | 19 (5.41) | 2 (1.94) | |
| Carcinosarcoma | 13 (3.70) | 7 (6.80) | |
| Mixed | 19 (5.41) | 6 (5.83) | |
| Unknown | 32 (9.12) | 10 (9.71) | |
| Center | | | 0.2670 |
| Yale New | 160 (45.48) | 38 (36.89) | |
| Haven Hospital | | | |
| Italy #1 | 146 (41.60) | 51 (49.51) | |
| Italy #2 | 45 (12.82) | 14 (13.59) | |
| Follow up Time | 40.40 (33.57) | 36.02 (29.40) | 0.2324 |
| (std deviation) | | | |

Documented BRCA mutant EOC cases with known outcome were collected from the following two institutions: (1) the YNHH (n=17) and (2) the City of Hope Comprehensive Cancer Center (n=62) (Table 19).

TABLE 19
Clinicopathologic parameters for BRCA mutant EOC patients.

| | Variable name | | |
|---|---|---|---|
| | Non-variant | KRAS-variant | P |
| | (n = 69) | (n = 10) | value |
| Age | 52.77 (10.20) | 52.60 (12.47) | 0.9623 |
| Stage | | | 0.1771 |
| I | 5 (7.25) | 2 (20.00) | |
| II | 8 (11.59) | 2 (20.00) | |
| III | 51 (73.91) | 5 (50.00) | |
| IV | 5 (7.25) | 1 (10.00) | |
| Grade | | | 0.5275 |
| Well | 2 (2.90) | 1 (10.00) | |
| differentiated | | | |
| Moderately | 13 (18.84) | 1 (10.00) | |
| differentiated | | | |
| Poorly | 49 (71.01) | 8 (80.00) | |
| differentiated | | | |
| Unknown | 5 (7.25) | 0 (0.00) | |
| Histology * | | | 0.9913 |
| Serous | 8 (11.59) | 1 (10.00) | |
| Endometrioid | 2 (2.90) | 0 (0.00) | |
| Undifferentiated | 1 (1.45) | 0 (0.00) | |
| Clear Cell | 1 (1.45) | 0 (0.00) | |
| Mucinous | 1 (1.45) | 0 (0.00) | |
| Carcinosarcoma | 1 (1.45) | 0 (0.00) | |
| Mixed | 1 (1.45) | 0 (0.00) | |
| Unknown | 54 (78.26) | 9 (90.00) | |
| BRCA status | | | 0.7206 |
| BRCA 1 | 51 (73.91) | 7 (70.00) | |
| BRCA 2 | 18 (26.09) | 3 (30.00) | |
| Center | | | 0.6808 |
| Yale New | 16 (23.19) | 1 (10.00) | |
| Haven Hospital | | | |
| City of Hope | 53 (76.81) | 9 (90.00) | |

* Histology information was not available for City of Hope patients

As not all stage 1 ovarian cancer patients receive adjuvant chemotherapy, when substage information was not available for patients with stage 1 tumors, these patients were excluded from the analysis. Otherwise, stage 1B and 1C tumors were included with stages 2-4. To minimize inadvertent inclusion of borderline tumors, tumors with an unknown grade were excluded from this analysis. For women treated with neoadjuvant chemotherapy, the date of pathological diagnosis was considered the start date of treatment. For women treated with adjuvant chemotherapy, the date of surgery was considered the start date of treatment. A total of 386 patients with wild-type BRCA or not tested for BRCA mutations and 79 patients with documented BRCA mutations fit the above-described parameters and were included in the two survival analyses.

Neoadjuvant chemotherapy cohort. Women with EOC who received neoadjuvant platinum-based chemotherapy followed by cytoreductive surgery at the YNHH between 1996 and 2010 were identified on an International Review Board-approved protocol (n=125) (Table 20). This cohort of patients received chemotherapy as a primary treatment due to tumor burden that was too extensive for optimal surgical debulking at presentation. After chemotherapy, patients underwent cytoreductive surgery and additional adjuvant treatment. Only patients treated with four or more cycles of neoadjuvant platinum-containing combinations were included in this analysis (n=116). Optimal cytoreduction was defined as residual disease measuring <1 cm remaining after surgery, whereas suboptimal cytoreduction was defined as residual disease measuring ≧1 cm at the completion of surgery. Only women operated on at Yale by the same group of surgeons were included to avoid bias in surgical skill as a factor impacting residual disease.

TABLE 20
Clinicopathologic parameters of patients
receiving neoadjuvant chemotherapy.

| | Variable name | | |
|---|---|---|---|
| | Non-variant | KRAS-variant | p |
| | (n = 97) | (n = 28) | value |
| Age (standard | 64.30 (12.12) | 62.57 (13.33) | 0.5170 |
| deviation) | | | |
| Ethnicity | | | 0.5889 |
| Caucasian | 90 (92 78) | 27 (96.43) | |
| Other or unknown | 7 (7.21) | 1 (3.57) | |
| Stage | | | 0.0175 |
| II | 1 (1.03) | 0 (0.00) | |
| III | 41 (42.27) | 4 (14.29) | |
| IV | 51 (52.58) | 23 (82.14) | |
| Unknown | 4 (4.12) | 1 (3.57) | |
| Grade | | | 0.1308 |
| Well | 2 (2.06) | 0 (0.00) | |
| differentiated | | | |
| Moderately | 13 (13.40) | 0 (0.00) | |
| differentiated | | | |
| Poorly | 68 (70.10) | 25 (89.29) | |
| differentiated | | | |
| Unknown | 14 (14.43) | 3 (10.71) | |
| Histology | | | 0.8176 |
| Serous | 73 (75.26) | 19 (67.86) | |
| Endometrioid | 2 (2.06) | 0 (0.00) | |
| Undifferentiated | 2 (2.06) | 0 (0.00) | |
| Clear Cell | 4 (4.12) | 2 (7.14) | |
| Mucinous | 1 (1.03) | 0 (0.00) | |
| Carcinosarcoma | 1 (1.03) | 1 (3.57) | |
| Mixed | 6 (6.19) | 3 (10.71) | |
| Unknown | 8 (8.25) | 3 (10.71) | |
| Neoadjuvant | | | 0.2765 |
| Chemotherapy | | | |
| Carboplatin/ | 85 (87.63) | 21 (75.00) | |
| Paclitaxel | | | |
| Carboplatin/ | 1 (1.03) | 1 (3.57) | |
| Taxotere | | | |
| Carboplatin/ | 7 (7.22) | 5 (17.86) | |
| Cyclophosphamide | | | |
| Other | 4 (4.12) | 1 (3.57) | |
| Neoadjuvant | | | 0.3502 |
| cycles completed: | | | |
| 2 | 2 (2.06) | 0 (0.00) | |
| 3 | 4 (4.12) | 2 (7.14) | |
| 4 | 18 (18.56) | 2 (7.14) | |
| 5 | 4 (4.12) | 4 (14.29) | |
| 6 | 64 (65.98) | 20 (71.43) | |
| 7 | 3 (3.09) | 0 (0.00) | |
| 9 | 1 (1.03) | 0 (0.00) | |
| Unknown | 1 (1.03) | 0 (0.00) | |
| Follow up time | 30.53 (25.45) | 36.54 (36.06) | 0.3229 |

Patients for analysis of platinum resistance. Platinum resistance was defined as progression-free survival of <6 months from the completion of platinum-containing adjuvant chemotherapy to the date of recurrence. The progression-free survival interval was available from women from Italy #1, Italy #2 and the YNHH patients (n=291). Table 21 describes the clinicopathological parameters of these patients.

TABLE 21
Clinicopathologic parameters for platinum resistance analysis.

| | Variable name | | |
|---|---|---|---|
| | Non-variant | KRAS-variant | p |
| | (n = 225) | (n = 66) | value |
| Age | 58.66 (11.70) | 56.11 (10.16) | 0.1129 |
| Stage: | | | 0.9652 |
| I | 41 (18.22) | 10 (15.15) | |
| II | 19 (8.44) | 6 (9.09) | |
| III | 142 (63.11) | 43 (65.15) | |
| IV | 22 (9.78) | 7 (10.61) | |
| Unknown | 1 (0.44) | 0 (0.00) | |
| Grade: | | | 0.0728 |
| Well | 18 (8.00) | 12 (18.18) | |
| differentiated | | | |
| Moderately | 42 (18.67) | 7 (10.61) | |
| differentiated | | | |
| Poorly | 150 (66.67) | 44 (66.67) | |
| differentiated | | | |
| Unknown | 15 (6.67) | 3 (4.55) | |
| Histology: | | | 0.6319 |
| Serous | 114 (50.67) | 31 (46.97) | |
| Endometrioid | 33 (14.67) | 10 (15.15) | |
| Undifferentiated | 27 (12.00) | 7 (10.61) | |
| Clear Cell | 14 (6.22) | 8 (12.12) | |
| Other | 37 (16.44) | 10 (15.15) | |
| Platinum response: | | | 0.0340 |
| Sensitive | 208 (92.44) | 55 (83.33) | |
| Resistant | 17 (7.56) | 11 (16.67) | |
| Cytoreductive | | | 0.4808 |
| surgery: | | | |
| Optimal | 129 (57.33) | 38 (57.58) | |
| cytoreduction | | | |
| (<1 cm residual | | | |
| disease) | | | |
| Suboptimal | 89 (39.56) | 28 (42.42) | |
| cytoreduction | | | |
| (>1 cm residual | | | |
| disease) | | | |
| Unknown | 7 (3.11) | 0 (0.00) | |
| Center | | | 0.2808 |
| Yale New | 55 (24.44) | 10 (15.15) | |
| Haven Hospital | | | |
| Italy #1 | 137 (60.89) | 46 (69.70) | |
| Italy #2 | 33 (14.67) | 10 (15.15) | |
| Follow up Time | 39.08 (24.97) | 36.47 (26.81) | 0.4635 |

Detection of the KRAS variant. DNA was isolated using standard methods from tumor, blood or saliva. The KRAS variant does not appear to be somatically acquired nor does it require a loss of heterozygosity (Chin L J, et al. (2008). Cancer Res 68: 8535-8540); hence, blood and saliva, for example, are appropriate to test and the results are identical regardless of the tissue tested. The KRAS variant allele was detected using a primer specific to the KRAS variant and a TaqMan (Applied Biosystems, Foster City, Calif., USA) PCR assay on all samples. Genotyping was performed at the YNHH, except for on samples from COH, for which the genotyping was performed in their facility. Less than 3% of populations carry 2 copies of the KRAS variant (Chin L J, et al. (2008). Cancer Res 68: 8535-8540). As such, patients who carried at least one copy of the KRAS variant allele were classified as KRAS-variant carriers.

Gene expression analysis of EOC with and without the KRAS variant. Gene expression in fresh-frozen tumor samples obtained from 16 patients (9 non-variant and 7 KRAS variant) was profiled on the Affymetrix GeneChip Human Genome U133 Plus 2.0 platform (Affymetrix, Santa Clara, Calif., USA). All samples were from high-grade serous epithelial ovarian tumors that were stage IIIC or IV. Images were processed with the MASS algorithm and probes that were judged absent in at least 75% of the samples were removed. Intensity values were log transformed and quantile normalized. Differential gene expression was assessed in samples obtained from patients over 52 years of age (n=6 non-variant and 4 KRAS variant) using a linear model and empirical Bayesian error moderation as implemented in the LIMMA package for R statistical software (R Foundation for Statistical Computing, publicly available at www.r-project.org) (Smyth G. (2005). Limma. in Gentleman R, et al. (eds) Bioinformatics and Computational Biology Solutions using R and Bioconductor. Springer: New York, pp. 397-420).

Association of published results with the KRAS variant in this data set was assessed using a signature approach to reduce cross-platform effects (Paranjape T, et al. (2011). Lancet Oncol 12: 377-386). In brief, signature scores were computed as Pearson's correlation between the respective signature vector of gene contributions and each sample's expression profile for these genes. Differences between signature scores in KRAS-variant and non-variant EOC samples were assessed using the paired Kolmogorov-Smirnov test. Unless otherwise indicated, gene lists from the respective publications were used as signature vectors. Data from the study by Peters et al. (Mol Cancer Ther 4: 1605-1616) were obtained from the Gene Expression Omnibus (GSE1926) and re-analyzed to generate a signature from the 50 most significantly differentially expressed genes between platinum sensitive and resistant samples.

Chemosensitivity and cell viability assays. The activity of drugs alone or in combination was determined by a high-through-put CellTiter-Blue cell viability assay. For these assays, 1.2×10³ cells were plated in each well of 384-well plates using a Precision XS liquid handling station (Bio-Tek Instruments Inc., Winooski, Vt., USA) and allowed to attach overnight with incubation at 37° C., 5% CO₂. Using the liquid handling station, all drugs were serially diluted 2:3 or 1:2 in media, and 5 μl of these dilutions were added to appropriate wells at indicated times. Four replicate wells were used for each drug concentration and an additional four control wells received a diluent control without drug. At the end of the incubation period with drugs, 5 μl CellTiter-Blue reagent (Promega Corp., Madison, Wis., USA) was added to each well. Cell viability was assessed by the ability of the remaining viable cells to bioreduce resazurin to resorufin. The fluorescence of resorufin (579 nm Ex/584 nm Em) was measured using a Synergy 4 microplate reader (Bio-Tek Instruments Inc.). The fluorescence data were transferred to Microsoft Excel (Microsoft) to calculate the percentage viability relative to the four replicate cell wells that did not receive the drug. IC50s were determined using a sigmoidal equilibrium model regression using XLfit version 5.2 (ID Business Solutions Ltd). The IC50 was defined as the concentration of drug required for a 50% reduction in growth/viability. All experiments were carried out a minimum of three times.

Targeting the KRAS variant. Small-interfering RNA sequences were designed to target the KRAS-variant sequence by placing the single-nucleotide polymorphism at varying positions of the 6 nucleotides at the 5′ end of the siRNA guide strand corresponding to the so-called ‘seed sequence’. Blast searches were performed to minimize cross-reactivity. In some of the siRNA sequences, DNA nucleotides were introduced to optimize thermoenergetic features for preferred incorporation of the guide strand into the argonaute effector complex or to increase specificity for the variant.

Small-interfering RNA guide strand sequences used in the experiments are as follows (lower case=RNA, upper case=DNA; GS=guide strand, PS=passenger strand):

| | | |
|---|---|---|
| 2-1 GS | ugcaucacuugaggucaggag | (SEQ ID NO: 23) |
| 2-1 PS | ccugaccucaagugaugcacc | (SEQ ID NO: 24) |
| 2-3 GS | TGCATCACuugaggucaggag | (SEQ ID NO: 25) |

(passenger strand same as 2-1)

| | | |
|---|---|---|
| 3-2 GS | ucaucacuugaggucaggagu | (SEQ ID NO: 26) |
| 3-2 PS | uccugaccucaagugaugcac | (SEQ ID NO: 27) |

The negative control used was purchased from Qiagen (Valencia, Calif., USA) (AllStars Negative-Control siRNA). Knockdown efficiency and specificity to the KRAS variant of these sequences were confirmed using a dual luciferase assay (see WO/2009/155100, the contents of which are incorporated herein by reference). Oligonucleotide combinations were annealed using standard conditions and then transfected into cells using standard protocols. Cell survival was assayed using MTT assays and experiments were conducted in quadruplicate, and repeated in four independent experiments for all lines. Cell lysates were collected 72 hours after transfection and KRAS protein levels measured by western analysis using a probe specific to KRAS as described previously (Chin L J, et al. (2008). Cancer Res 68: 8535-8540).

Statistics. To assess the significance of demographic variables, a χ² test or a two-sided Fisher's exact test was used for categorical variables. A t-test was used for continuous variables, such as age. The overall survival time of KRAS-variant and wild-type patients was compared using the Kaplan-Meier method (Kaplan E and Meier P. (1958). J Am Stat Assoc 53: 457-481), and the statistical significance of the survival curves was determined by the log-rank test (Mantel N. (1966). Cancer Chemother Rep 50: 163-170). A Cox proportional hazards regression model (Cox D. (1972). J R Stat Soc 34: 187-220) was used to assess the impact of the KRAS variant and demographic and prognostic variables (such as age, stage, grade and histology) on overall survival. Multivariate logistic regression analyses (Cox D. (1970). The Analysis of Binary Data. Methuen, London) were used to determine the impact of the KRAS variant and other demographic and prognostic factors on the probability of suboptimal cytoreduction. Multivariate logistic regression analyses (Cox D. (1970). The Analysis of Binary Data. Methuen, London) were used to assess the association of the KRAS variant and other prognostic factors on the probability of platinum resistance. All statistical analyses were performed using SAS 9.1.3 (SAS Institute Inc., Cary, N.C., USA) and in R 2.12.1 (R Foundation for Statistical Computing)

### Data and Results

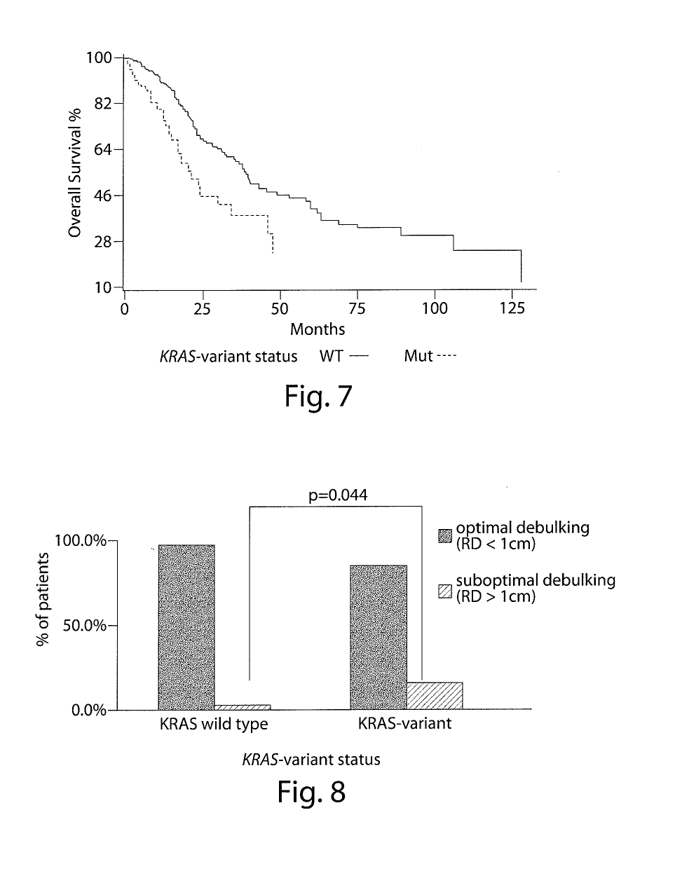

The association of the KRAS variant with overall survival in 454 EOC patients either tested and negative or untested for deleterious BRCA mutations was evaluated. When the entire cohort was considered, the KRAS variant did not predict worse survival by Kaplan-Meier analysis. Because the KRAS variant is most strongly associated with postmenopausal ovarian cancer (Chin L J, et al. (2008). Cancer Res 68: 8535-8540), survival in women over 52 years of age (n=279) were evaluated. Over and including 52 years of age is considered to be an appropriate surrogate for menopausal status. By Kaplan-Meier analysis, survival was significantly reduced in postmenopausal KRAS-variant EOC patients (n=59) compared with non-variant EOC patients (n=220, FIG. 7, logrank P=0.0399, non-KRAS-variant survival median 60 months, KRAS-variant survival median 34 months). When other variables including age, stage, grade, histology and treatment center were included with KRAS-variant status in a multivariate Cox proportional hazards regression model, the KRAS variant was a statistically significant predictor of reduced overall survival for postmenopausal women with EOC (Table 22); the hazard ratio for the KRAS variant was 1.67 (95% confidence interval: 1.09-2.57, P=0.019).

TABLE 22
The KRAS variant is associated with reduced survival in postmenopausal
(>52 years of age) ovarian cancer patients (n = 279).

| Variable | HR | 95% CI | P-value |
|---|---|---|---|
| KRAS status | 1.671 | 1.087-2.568 | 0.0192 |
| Age | 1.025 | 1.002-1.049 | 0.0307 |
| Stage | 1.380 | 1.185-1.607 | <0.0001 |
| Grade | 1.341 | 0.912-1.972 | 0.1360 |
| Histology | 0.970 | 0.900-1.045 | 0.4168 |
| Center (Non-Yale | 1.868 | 1.438-2.427 | <0.0001 |
| vs Yale) | | | |

Abbreviations: CI, confidence interval; HR, hazard ratio obtained from Cox proportional hazards multivariate analysis.
Studies included the Yale New Haven Hospital, Italy #1, Italy #2.

The association of the KRAS variant with survival in a separate cohort of EOC patients carrying deleterious BRCA1 or BRCA2 mutations (n=79) was evaluated. EOC patients carrying BRCA mutations were statistically significantly younger than EOC patients without BRCA mutations (52.7 vs 60.8 years of age, P<0.0001). In addition, EOC patients with BRCA mutations had a significantly longer median survival by multivariate analysis controlling for age, stage, grade and histology than did EOC patients without BRCA mutations (120 vs 52 months, P=0.0036). There was no significant difference in survival between EOC patients with BRCA mutations with or without the KRAS variant in a multivariate analysis using a multivariate Cox proportional hazards regression model (Table 23, KRAS-variant hazard ratio=0.75, 95% confidence interval: 0.21-2.72, P=0.66). In this study, there were too few patients to evaluate the impact of the KRAS variant on survival in postmenopausal EOC patients with deleterious BRCA mutations.

TABLE 23
The KRAS-variant and overall survival in EOC patients
with deleterious BRCA mutations (n = 79).

| Variable | HR | 95% CI | p value |
|---|---|---|---|
| KRAS status | 0.75 | 0.21-2.72 | 0.66 |
| Age | 1.01 | 0.98-1.05 | 0.45 |
| Stage | | | 0.0005 |
| Stage III vs. | 14.79 | 1.87-117.29 | 0.01 |
| Stage I and II | | | |
| Stage IV vs. | 69.98 | 7.00-699.87 | 0.0003 |
| Stage I and II | | | |
| Grade | | | |
| Grade 2 and 3 vs. | 4.32 | 1.29-14.46 | 0.02 |
| Grade 1 | | | |
| Center (non Yale | 0.66 | 0.23-1.87 | 0.43 |
| vs. Yale) | | | |

HR: hazards ratio obtained from Cox proportional Hazards multivariate analysis
CI: confidence interval
Studies Included: Yale New Haven Hospital, City of Hope

To explain the reduced survival in postmenopausal KRAS variant-positive EOC patients, the association of KRAS-variant positivity with response to platinum-based chemotherapy was evaluated. Platinum-based chemotherapy is the standard first-line chemotherapy in the treatment of EOC. First, all women with EOC who were treated at the Yale-New Haven Hospital (YNHH) with neoadjuvant platinum-containing chemotherapy followed by surgical cytoreduction (n=116) were evaluated. Residual disease after surgery (cytoreduction) was used as a surrogate marker of patient response to chemotherapy. It was determined that 15.4% of KRAS-variant patients (n=26) were suboptimally cytoreduced (41 cm of residual disease after surgery), compared with only 3.33% of non-variant patients (n=90) (FIG. 8, P=0.044). The KRAS variant was also significantly associated with suboptimal cytoreduction after neoadjuvant chemotherapy and surgery in a multivariate logistic regression model controlling for age, stage, grade and histology (Table 24, odds ratio=9.36, 95% confidence interval: 1.34-65.22, P=0.024).

TABLE 24
The KRAS-variant predicts suboptimal debulking after neoadjuvant
chemotherapy (n = 116).

| KRAS-variant | Univariate | | | Multivariate³ | | |
|---|---|---|---|---|---|---|
| Genotype | OR¹ | 95% CI² | p | OR | 95% CI | p |
| All | | | | | | |
| Wild-type | 1.00 | | | 1.00 | | |
| (n = 90) | | | | | | |
| Variant | 5.27 | 1.10-25.30 | 0.0377 | 9.36 | 1.34-65.22 | 0.0240 |
| (n = 26) | | | | | | |

¹OR: odds ratio obtained from logistic regression
²CI: confidence interval
³Multivariate: adjusted for age, stage, grade, histology, type of chemotherapy regimen, and numbers of cycles received prior to surgery.

To determine whether the cause of poor response to neoadjuvant platinum-based chemotherapy seen in KRAS-variant EOC patients was due to resistance to platinum chemotherapy, platinum resistance in all EOC patients treated adjuvantly with platinum chemotherapy without documented BRCA mutations with available response data (n=291) were evaluated. It was determined that platinum resistance (defined in this example as disease recurrence within 6 months of receiving platinum-based chemotherapy) was significantly more likely in KRAS variant-positive EOC patients than in non-KRAS variant EOC patients (16.67 vs 7.56%, P=0.034). The KRAS variant was a statistically significant predictor for platinum resistance for EOC patients of all ages in a multivariate logistic regression analysis controlling for residual disease remaining after cytoreductive surgery, stage, histology, age and grade (Table 25, odds ratio=3.18, 95% confidence interval: 1.31-7.72, P=0.0106).

TABLE 25
The KRAS variant is associated with platinum resistance.

| KRAS variant | Univariate | | | Multivariate^{a} | | |
|---|---|---|---|---|---|---|
| genotype | OR | 95% CI | P-value | OR | 95% CI | P-value |
| All | | | | | | |
| Non-variant | 1.00 | | | 1.00 | | |
| (n = 225) | | | | | | |
| Variant | 2.45 | 1.08-5.53 | 0.0313 | 3.18 | 1.31-7.72 | 0.0106 |
| (n = 66) | | | | | | |

Abbreviations:
CI: confidence interval;
OR: odds ratio obtained from logistic regression.
Studies: Yale, Italy #1, Italy #2.
^{a}Multivariate adjusted for age, stage, grade, histology, residual disease after cytoreductive surgery and treatment center.

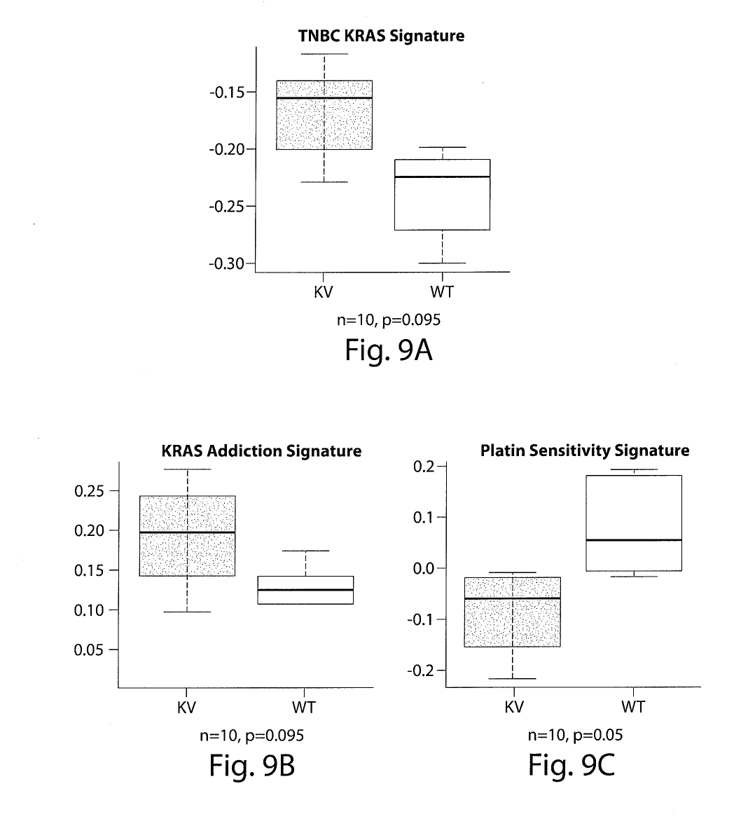

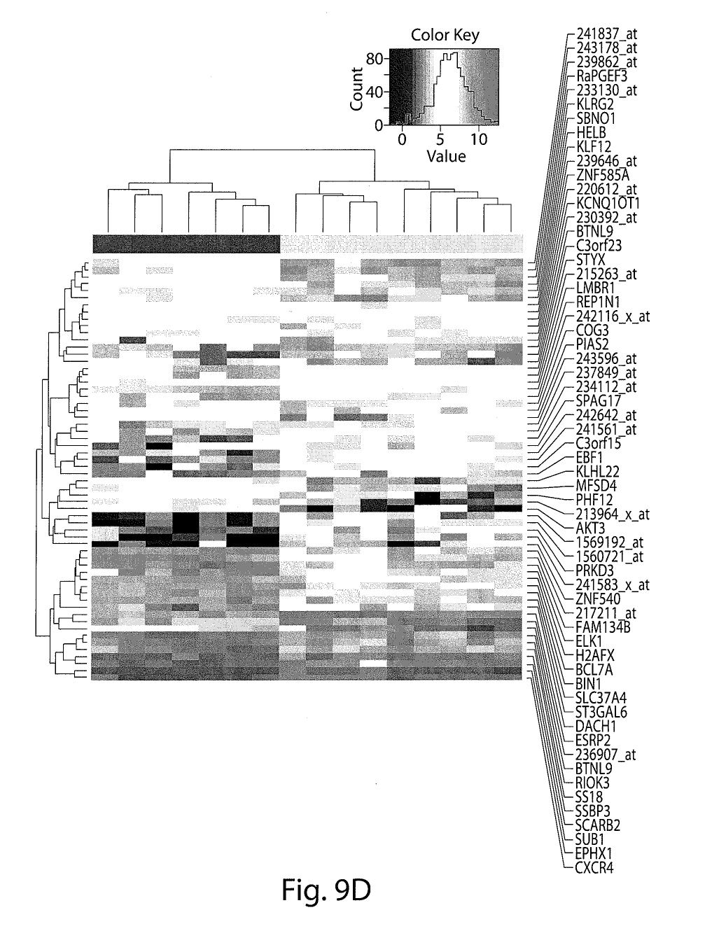

Gene expression studies were performed on a small cohort of ovarian cancer patients who had fresh-frozen tissue available (Brescia cohort), and compared between seven serous EOC samples with the KRAS variant and nine without the KRAS variant (n=16). Within this cohort, in postmenopausal EOC patients over 52 years of age with EOC (n=10), a gene signature previously found to be associated with KRAS variant-associated TNBC (Paranjape T, et al. (2011). Lancet Oncol 12: 377-86) was also upregulated in KRAS variant-associated EOC (FIG. 9a). Similar to the previous analysis in TNBC, overexpression of KRAS-associated downstream pathways in EOC KRAS-variant tumors was discovered, which is consistent with ‘KRAS addiction’ (Singh A, et al. (2009). Cancer Cell 15: 489-500) (FIG. 9b).

Using previous analyses of gene expression data identifying platinum-resistant vs sensitive signatures (Peters D, et al. (2005). Mol Cancer Ther 4: 1605-1616), it was determined that KRAS-variant EOC samples had a lower carboplatin sensitivity signature compared with non-variant EOC samples (FIG. 9c). In agreement with findings showing that the activation of the AKT pathway was frequently involved in platinum resistance, it was determined that AKT3 was one of the most significantly upregulated transcripts in KRAS-variant EOC tumors (FIG. 9d).

Although miRNA expression data were not available on tumor samples, the expression of let-7b miRNA in two cell lines with the KRAS variant (BG-1 and IGROV1) was compared with the expression of let-7b in a non-KRAS variant line (CAOV3). The expression of let-7b miRNA is altered in KRAS variant-positive lung tumors (Chin L J, et al. (2008). Cancer Res 68: 8535-8540) and triple-negative breast tumors (Paranjape T, et al. (2011). Lancet Oncol 12: 377-386).

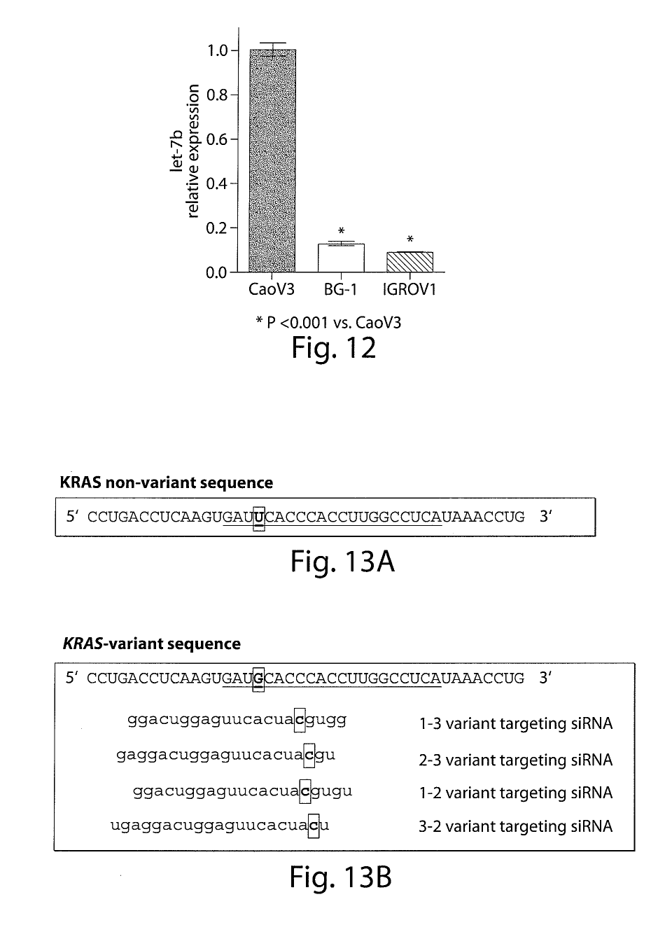

It was determined that let-7b was statistically significantly lower in cells with the KRAS variant (FIG. 12).

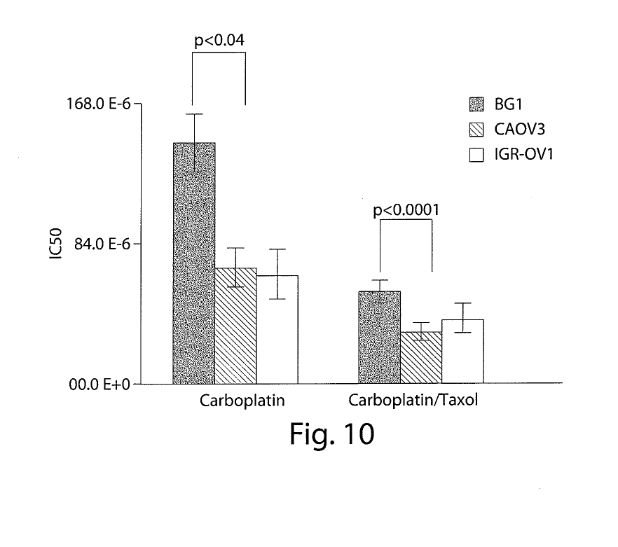

To confirm altered chemosensitivity in the presence of the KRAS variant, EOC cell lines with and without the KRAS variant were used to test their sensitivity to different chemotherapeutic agents. For example, a cell line that is KRAS variant positive/BRCA wild-type (BG1), a non-variant/BRCA wild-type cell line (CAOV3) and a cell line KRAS-variant positive/BRCA1 mutant (IGR-OV1) were tested. It was determined that the KRAS-variant line, BG1, was statistically significantly resistant to carboplatin (P<0.04) and carboplatin/paclitaxel combination chemotherapy (P<0.0001) compared with CAOV3, the cell line without the KRAS variant. In contrast, IGROV1, the cell line with the KRAS variant and a deleterious BRCA1 mutation, was not resistant to these agents when compared with CAOV3 (FIG. 10). These results agree with corresponding clinical results demonstrating that the KRAS variant is associated with platinum resistance, but not in the presence of deleterious BRCA mutations.

Additionally, agents frequently used as second line therapy for patients who have failed carboplatin/paclitaxel chemotherapy were evaluated. These second line therapeutic agents included doxorubicin, topotecan and gemcitabine. The KRAS-variant line, BG1, was significantly resistant to each of these agents compared with CAOV3, the nonvariant cell line (Table 26).

TABLE 26
Chemosensitivity in a KRAS-variant cell
line (BG1) vs a non-variant line (CAOV3).

| | Gemcitabine | Doxorubicin | Topotecan | RSE |
|---|---|---|---|---|
| BG1 | 30.4 10\hat{ }6 | 307.5 10\hat{ }9 | 161.8 10\hat{ }9 | 21.69 |
| CAOV3 | 2.2 10\hat{ }9 | 75.9 10\hat{ }9 | 30.8 10\hat{ }9 | 19.67 |

Abbreviation: RSE, relative standard error which is the s.e. divided by the mean and expressed as a percentage.
Numbers are IC50 values from a minimum of four separate experiments.
Differences are statistically significant (P < 0.01), indicating that the KRAS-variant line is more resistant to these agents.

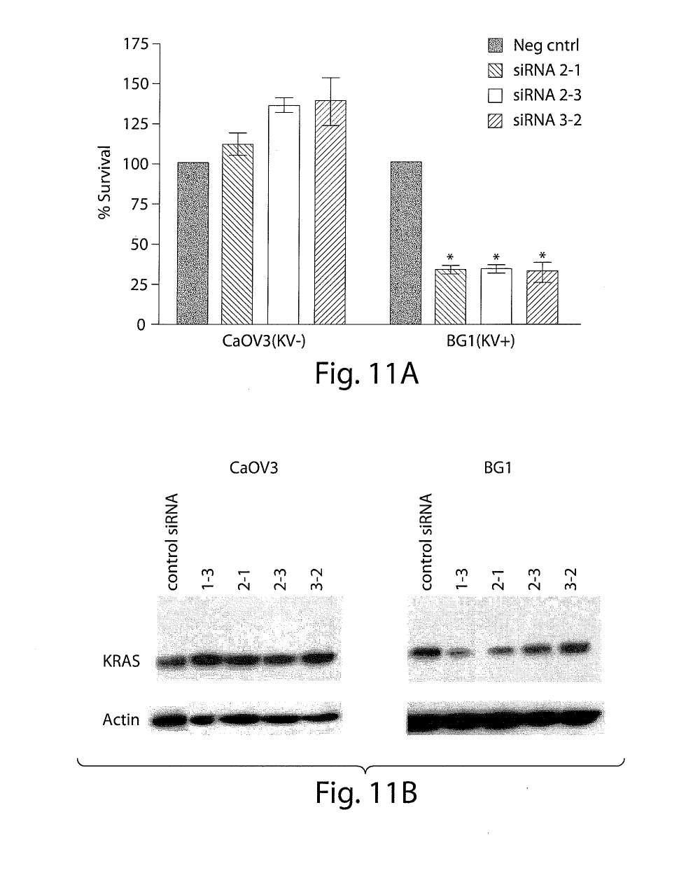

Because the data presented herein demonstrate a continued use of KRAS signaling in KRAS variant-associated tumors, the impact of directly targeting the KRAS-variant was evaluated. Small-interfering RNA (siRNA)/miRNA-like complexes were designed to directly bind the altered allele in KRAS variant transcripts, but not bind to non-KRAS-variant transcripts (FIG. 13). It was determined that transfecting these oligonucleotide duplexes that target the KRAS variant caused a statistically significant decrease in cell survival in the KRAS variant carrying BG1 cell line (P<0.001), but had no effect in CAOV3 (FIG. 11a) or SKOV3, two non-variant EOC cell lines. This result is concordant with a moderate decrease in KRAS protein levels by western blot in BG1, but not in CAOV3 (FIG. 11b) or SKOV3 after treatment.

### Example 4

### The KRAS Variant as a Prognostic Biomarker in Early-Stage Colorectal Cancer (CRC)

### Materials and Methods

Study population. Until 1994, 925 incident CRC cases (ICD-O:153.0-154.1) were identified within the Netherlands Cohort Study on diet and cancer (NLCS) which started in 1986 with 120,852b healthy persons between 55 and 69 years. Incident cancer cases were identified by linkage with the Netherlands Cancer Registry (NCR) and PALGA, a nationwide registry of histopathology and cytopathology (Van den Brandt P A, et al. Int J Epidemiol. 1990; 19(3): 553-8). The NLCS has been described in detail elsewhere (Van den Brandt P A, et al. J Clin Epidemiol. 1990; 43(3): 285-95. 815 CRC cases could be linked to PALGA and paraffin-embedded tumor tissue was collected from 54 pathology registries throughout the Netherlands. A sufficient amount of good quality DNA was extracted for 734 (90%) cases (Brink M, et al. Carcinogenesis. 2003; 24(4): 703-10). At baseline, a subcohort of 5000 healthy persons was randomly sampled from the entire cohort to estimate personyears at risk of the cohort through biennial follow-up of vital status. For 1,886 persons, DNA from buccal swabs was available for KRAS variant genotyping.

Data collection. Information on tumor localization, stage, differentiation grade, incidence date and treatment in the 3 months after diagnosis, was available through the NCR. Vital status until May 2005 was retrieved from the Central Bureau of Genealogy and the municipal population registries and could be obtained for all 734 cases. Causes of death were retrieved through linkage with Statistics Netherlands. CRC-related deaths were defined as deaths as a result of a carcinoma in the colon, rectosigmoid, rectum, gastro-intestinal tract (non-specific) or liver metastases. In the case of gastro-intestinal (non-specified) or liver metastases, information from NCR and PALGA was used to eliminate the possibility of another primary cancer as cause of death.

DNA isolation and KRAS-variant determination. A 5 μm section of each tumor tissue block was stained with haematoxylin and eosin and revised by a pathologist. Five sections of 20 μM were deparaffinated and DNA was extracted using the Puregene® DNA isolation kit (Gentra systems) according to the manufacturers' instructions. In brief, cell lysis solution and proteinase K (20 mg/ml, Qiagen) were added to the tissue and incubated overnight at 55° C. DNA was extracted for 72 hours at 37° C., protein was removed, and DNA was precipitated using 100% 2-propanol. Finally, DNA was rehydrated in hydration buffer. Isolated DNA was amplified using TaqMan PCR assays designed specifically to identify the T or G allele (wild type and variant alleles, respectively) of the let-7 complementary site 6 (LCS6) within the 3′UTR of KRAS (Applied Biosciences). Although tumor DNA was used to assess genotype, it is well documented that the genotype of normal and tumor tissue is the same in KRAS variant allele carriers (Chin L J, et al. Cancer Res. 2008; 68(20): 8535-40).

KRAS and BRAF mutations were assessed by nested polymerase chain reaction (PCR) and direct sequencing (KRAS), and restriction fragment length polymorphism (BRAF) as described previously (Brink M, et al. Carcinogenesis. 2003; 24(4): 703-10; de Vogel S, et al. Carcinogenesis. 2008; 29(9): 1765-73). Promoter methylation of RASSF1A, O⁶-MGMT, CHFR and CIMP markers as proposed by Weisenberger (Weisenberger D J, et al. Nat. Genet. 2006; 38(7): 787-93) was assessed by chemical modification of genomic DNA with sodium bisulfite and methylation-specific PCR (MSP) (de Vogel Set al. Carcinogenesis. 2008; 29(9):1765-73; 26. Herman J G, et al. Proc Natl Acad Sci USA. 1996; 93(18): 9821-6; Derks S, et al. Cell Oncol. 2004; 26(5-6): 291-9). MSI status was determined using BAT-26, BAT-25, NR-21, NR-22 and NR-24 as described previously (Suraweera N, et al. Gastroenterology. 2002; 123(6): 1804-11). All assays were performed and analyzed while blinded to the main study endpoint, i.e. CRC-related death.

Statistical analyses. Cause-specific survival was defined as time from cancer diagnosis until CRC-related death or end of follow-up. Kaplan-Meier curves and log-rank tests were used to estimate the influence of the KRAS variant on cause-specific survival. HR and corresponding 95% CI were assessed by use of Cox proportional hazard models adjusted for potential confounders. Factors were considered possible confounders if they were known prognostic factors for CRC and influenced the crude HR by more than 10%. Confounders that were included were age at diagnosis (continuous), sex, tumor differentiation grade (well, moderate, poor, and undifferentiated), and location (proximal, distal, rectosigmoid, and rectum). The proportional hazard assumption was tested using the Schoenfeld residuals and the log (-log) hazards plots. Survival analyses were restricted to 10 years after diagnosis as CRC-related cause of death was unlikely after that point. Incidence rate ratios (RR) and 95% CI were estimated using Cox proportional hazards models. Standard errors were estimated using the robust Huber-White sandwich estimator to account for additional variance introduced by sampling from the cohort. All analyses were done with the statistical package STATA10.0.

### Data and Results

Patients in this study were more often male (55.6%), diagnosed with an early-stage tumor (62.0%) or a proximal or distal tumor (65.3%; Table 27). During follow-up, 41.4% of the patients died of CRC. The KRAS-LCS6 variant was detected in 14.0% of early-stage (stage I and II), in 19.2% of stage III and 21.4% of stage IV patients (P=0.160; P_{trend}=0.060). KRAS variant patients were more often diagnosed with advanced stage disease (47.5% versus. 36.9% in wild-type patients, P=0.046). Other statistically significant differences were not found between wild type and KRAS variant carriers for sex, age at diagnosis, differentiation grade, tumor location, MSI, or mutations in KRAS (Table 27), BRAF (P=0.640), or RASSF1A promoter CpG island methylation (P=0.423). As expected, patients with stage III or IV disease more often died from CRC (P<0.001) and more often had a poorly differentiated tumor (P<0.001). Advanced stage patients more often had a proximal (P=0.036) or MSS tumor (P=0.047) as compared with early-stage patients.

TABLE 27
Baseline characteristics for the total population, KRAS variant and wild type
carriers and early stage and advanced stage CRC cases within the NLCS on diet and cancer,
between 1986 and 1994, inclusively.

| | | | | KRAS-LCS6 | | | | | |
|---|---|---|---|---|---|---|---|---|---|
| | | | | variant | | Early-stage | | | |
| | | | KRAS-LCS6 | G-allele | | (stage I and II) | | | |
| | | Overall | wild-type TT | (He + Ho) | P | CRC | Stage III | Stage IV | P |
| Total population, n (%) | | 734 (100) | 567 (83.6) | 111 (16.4) | | 409 (62.0) | 162 (27.6) | 69 (10.5) | |
| Sex [male, n (%)] | Male | 406 (55.6) | 308 (54.3) | 66 (59.5) | 0.320 | 219 (53.6) | 102 (56.0) | 33 (47.8) | 0.506 |
| Age at diagnosis | | 67.9 (4.3) | 67.9 (4.3) | 67.9 (4.4) | 0.885 | 68.0 (4.4) | 67.5 (4.1) | 68.5 (3.8) | 0.203 |
| (mean, SD) | | | | | | | | | |
| CRC-related death | Yes | 302 (41.4) | 230 (40.6) | 46 (42.2) | 0.761 | 95 (23.3) | 107 (8.8) | 65 (95.6) | <0.001 |
| (yes, n (%)) | | | | | | | | | |
| Cancer stage, n (%) | Early state | 409 (62.0) | 326 (63.1) | 53 (52.5) | | | | | |
| | (I and II) | | | | | | | | |
| | III | 182 (27.6) | 137 (26.5) | 33 (32.7) | | | | | |
| | IV | 69 (10.5) | 54 (10.4) | 15 (14.9) | 0.124 | | | | |
| Differentiation, n (%) | Well | 74 (11.5) | 58 (11.8) | 9 (8.7) | | 46 (12.7) | 13 (7.8) | 3 (5.0) | |
| | Moderate | 457 (71.0) | 354 (71.8) | 72 (69.9) | | 277 (76.5) | 109 (65.3) | 36 (60.0) | |
| | Poor | 106 (16.5) | 75 (15.2) | 21 (20.4) | | 37 (10.2) | 41 (24.6) | 20 (33.3) | |
| | Undifferentiated | 7 (1.1) | 6 (1.2) | 1 (1.0) | 0.532 | 2 (0.6) | 4 (2.4) | 1 (1.7) | <0.001 |
| Location, n (%) | Proximal | 239 (33.2) | 196 (35.4) | 34 (31.2) | | 128 (31.5) | 63 (34.8) | 33 (49.3) | |
| | Distal | 231 (32.1) | 177 (32.0) | 37 (33.9) | | 125 (30.7) | 61 (33.7) | 22 (32.8) | |
| | Rectosigmold | 80 (11.1) | 59 (10.6) | 11 (10.1) | | 53 (13.0) | 17 (9.4) | 5 (7.5) | |
| | Rectum | 169 (23.5) | 122 (22.0) | 27 (24.8) | 0.824 | 101 (24.8) | 40 (22.1) | 7 (10.5) | 0.036 |
| Molecular charateriatics, | MSS | 578 (87.3) | 463 (87.5) | 88 (84.6) | | 314 (84.9) | 149 (88.7) | 63 (95.5) | |
| n (%) | MSI | 84 (12.7) | 66 (12.5) | 16 (15.4) | 0.420 | 56 (15.1) | 19 (11.3) | 3 (4.5) | 0.047 |
| | CIMP+ | 167 (27.7) | 127 (24.5) | 34 (35.4) | | | | | |
| | CIMP | 436 (72.3) | 352 (73.5) | 62 (64.6) | 0.076 | | | | 0.121 |
| KRAS mutations, n (%) | Wild type | 464 (63.2) | 362 (63.8) | 69 (62.2) | | 263 (64.3) | 121 (66.5) | 39 (56.5) | |
| | KRAS mutated | 270 (36.8) | 205 (36.2) | 42 (37.8) | 0.736 | 146 (35.7) | 61 (33.5) | 30 (43.5) | 0.336 |
| KRAS variant | Wild type | 567 (83.6) | | | | 326 (86.0) | 137 (80.6) | 54 (78.3) | |
| | Variant He | 107 (15.8) | | | | 51 (13.5) | 32 (18.8) | 15 (21.7) | |
| | Variant Ho | 4 (0.6) | | | | 2 (0.5) | 1 (0.6) | | 0.298 |

Stage IV G-allele (KRAS variant) carriers were more likely to be female (66.7%; P=0.097) and to present with a proximal tumor (71.4%; P=0.004) as compared with G-allele (KRAS variant) carriers in other stages (Table 28).

TABLE 28
Baseline and molecular characteristics for early stage, stage III and IV patients
according to KRAS variant status.

| | | KRAS-LCS6 wild-type TT | | | | KRAS-LCS6 variant G-allele (He + Ho) | | | |
|---|---|---|---|---|---|---|---|---|---|
| | | Stage I and II | Stage III | Stage IV | P | Stage I and II | Stage III | Stage IV | P |
| Tolal population, | | 326 (63.1) | 137 (26.5) | 54 (10.4) | | 53 (52.5) | 33 (32.7) | 15 (14.9) | |
| n (%) | | | | | | | | | |
| T1 | | 43 (13.2) | 1 (0.7) | — | | 8 (15.1) | — | — | |
| T2 | | 95 (29.1) | 13 (9.6) | 3 (5.6) | | 17 (32.1) | 5 (15.6) | — | |
| T3 | | 174 (53.4) | 113 (83.1) | 41 (75.9) | | 27 (50.9) | 24 (75.0) | 12 (80.0) | |
| T4 | | 14 (4.3) | 9 (6.6) | 10 (185) | <0.001 | 1 (1.9) | 3 (9.4) | 3 (20.0) | 0.001 |
| Sex [male, n (%)] | Male | 173 (53.1) | 75 (54.7) | 28 (51.9) | 0.920 | 29 (54.7) | 22 (66.7) | 5 (33.3) | 0.097 |
| Age at diagnosis | | 68.0 (0.3) | 67.5 (0.4) | 68.5 (0.5) | 0.283 | 68.0 (0.6) | 67.5 (0.7) | 68.5 (1.2) | 0.756 |
| (mean, SD) | | | | | | | | | |
| CRC-related | Yes | 79 (24.2) | 79 (57.7) | 51 (96.2) | <0.001 | 8 (15.7) | 19 (57.6) | 14 (93.3) | <0.001 |
| death [yes, n (%)] | | | | | | | | | |
| Differentiation, | Well | 38 (13.2) | 9 (7.3) | 2 (4.4) | | 5 (10.6) | 2 (6.3) | 1 (7.1) | |
| n (%) | Moderate | 224 (77.8) | 77 (62.1) | 31 (67.4) | | 35 (74.5) | 24 (75.0) | 5 (35.7) | |
| | Poor | 25 (8.7) | 34 (27.4) | 12 (26.1) | | 6 (12.8) | 6 (18.8) | 8 (57.1) | |
| | Undifferentiated | 1 (0.4) | 4 (3.2) | 1 (2.2) | <0.001 | 1 (2.1) | — | — | 0.028 |
| Location, n (%) | Proximal | 107 (33.0) | 55 (40.2) | 23 (43.4) | | 15 (28.3) | 6 (18.8) | 10 (71.4) | |
| | Distal | 100 (30.9) | 42 (30.7) | 20 (37.7) | | 14 (26.4) | 17 (53.1) | 2 (14.3) | |
| | Rectosigmold | 40 (12.4) | 12 (8.8) | 4 (7.6) | | 8 (15.1) | 2 (6.3) | 1 (7.1) | |
| | Rectum | 77 (23.8) | 28 (20.4) | 6 (11.3) | 0.230 | 16 (30.2) | 7 (21.9) | 1 (7.1) | 0.004 |
| Molecular | MSS | 258 (85.2) | 113 (86.9) | 51 (100) | 0.013 | 40 (81.6) | 30 (93.8) | 12 (80.0) | 0.259 |
| characteristics, | CIMP+ | 72 (26.5) | 34 (28.3) | 16 (34.8) | 0.504 | 14 (30.4) | 8 (27.6) | 8 (57.1) | 0.126 |
| n (%) | | | | | | | | | |
| KRAS mutations, | KRAS mutated | 115 (35.3) | 44 (32.1) | 23 (42.6) | 0.393 | 20 (37.7) | 11 (33.3) | 7 (46.7) | 0.676 |
| n (%) | | | | | | | | | |

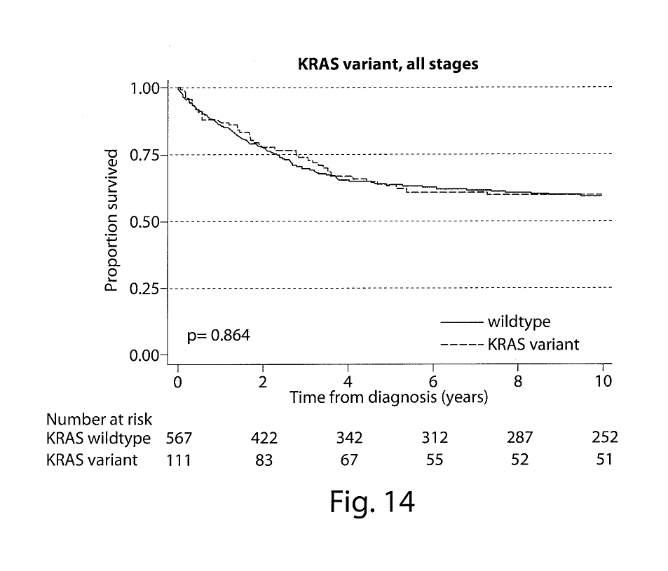

The KRAS variant is associated with better survival in early-stage CRC. A statistically significant difference was not observed in Kaplan-Meier analyses for the KRAS variant and cause-specific survival in the total population (log-rank test, P=0.864) (FIG. 14).

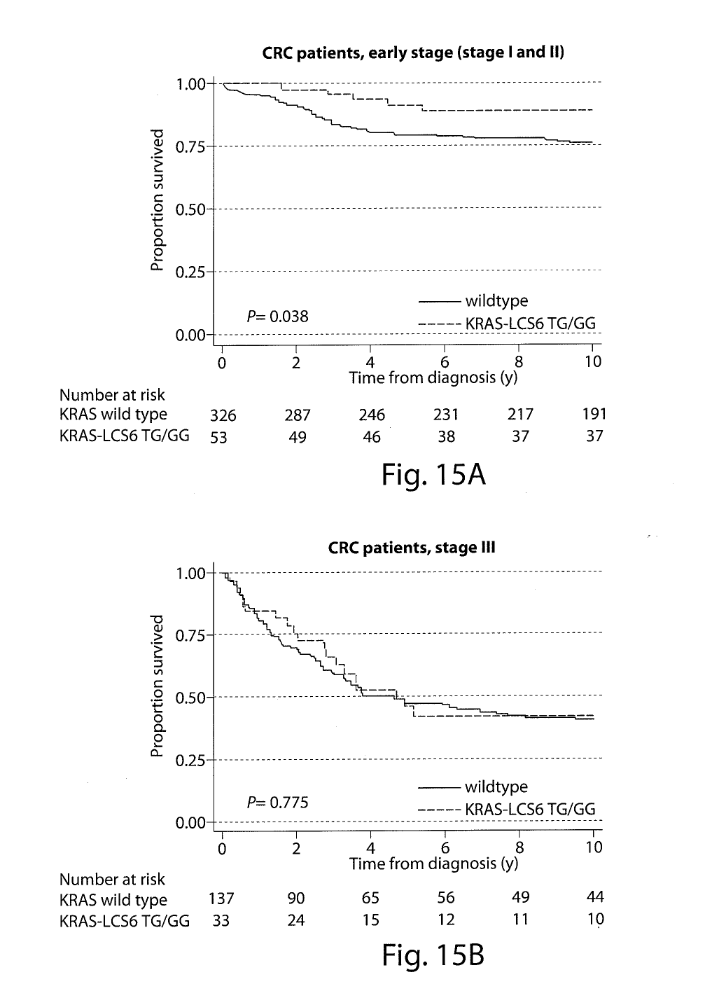

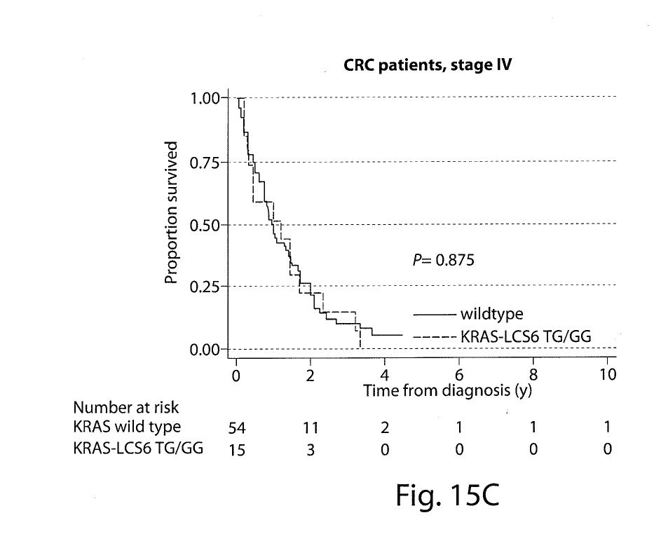

As survival depends on cancer stage, the analyses conducted were stratified for stage. Early-stage G-allele (KRAS variant) carriers showed a statistically significantly better survival as compared with wild-type cases (log-rank test, P=0.038; FIG. 15A). This difference was not observed for advanced stage cases (FIGS. 1B and C; log rank, P=0.775 and 0.875 for stage III and IV cases, respectively).

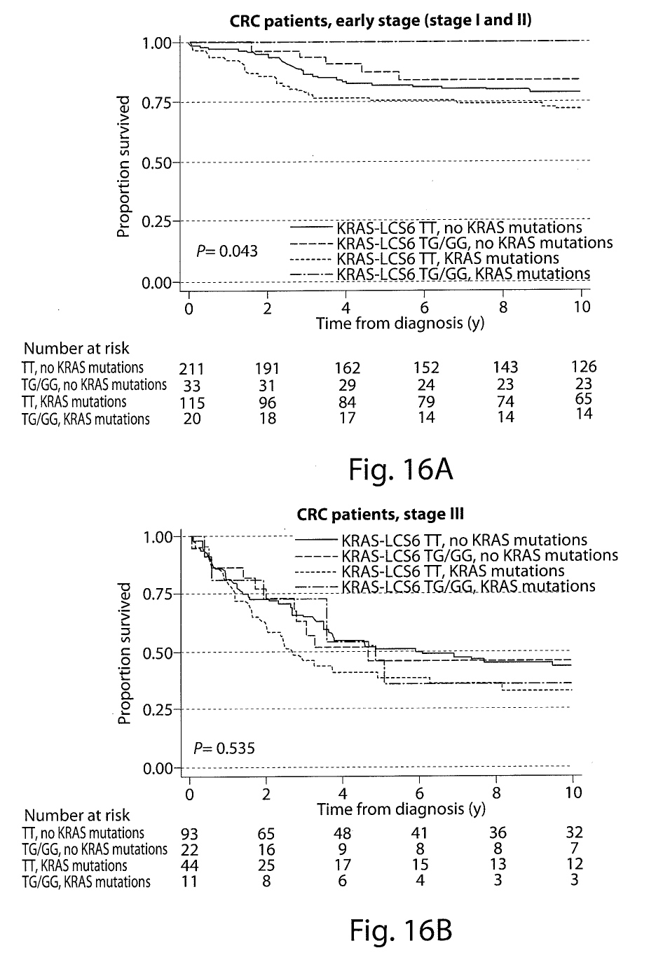

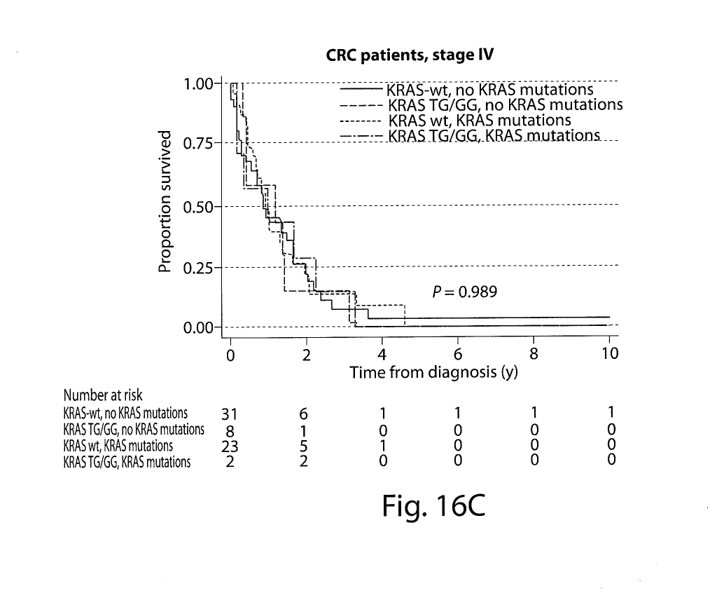

KRAS/BRAF mutation status enhances the association between the KRAS variant and survival. FIG. 16A shows Kaplan-Meier analyses for early-stage (stage I and II) CRC cases with the KRAS variant and KRAS mutations. None of the 20 G-allele (KRAS variant) carriers with KRAS mutations died due to CRC. KRAS wild-type patients had a poorer survival, especially if they had KRAS mutations (log-rank test, P=0.043; log-rank test KRAS-variant allele carriers with KRAS mutations compared with KRAS-variant allele carriers without KRAS mutations P=0.017). This discovery was independent of T stage; among 115 KRAS wild-type cases with KRAS mutations, only 5 (4%) were diagnosed as high-risk stage IIb (T4N0M0). Among G-allele (KRAS variant allele) carriers, no patients were diagnosed as stage IIb. For advanced stage patients, a survival difference was not found (FIGS. 16B and 16C, log-rank test, P=0.535 for stage III and P=0.989 for stage IV)). Results for stage III patients indicate that KRAS wild type patients with KRAS mutations have the worst prognosis. Subgroup analysis showed that the better outcome for early-stage KRAS variant carriers was found predominantly in stage II cases. Analyses stratified for T stage were not possible due to limited patient numbers.

BRAF mutated CRCs carrying the G-allele showed a similar better outcome, although this was not statistically significant (log-rank test, P=0.166) possibly due to small number of patients carrying both KRAS variant and KRAS mutations (9 patients). Similarly, G-allele (KRAS variant allele) carriers with aberrant RASSF1A promoter hypermethylation, another gene involved in the Ras pathway, had a better prognosis, although less statistically significant, as compared with wild-type carriers without RASSF1A hypermethylation (log-rank test, P=0.062). Analyses combining KRAS, BRAF, and RASSF1A status showed that early-stage G-allele (KRAS variant) carriers with additional alterations in KRAS, BRAF, or RASSF1A have a better prognosis (log-rank test, P=0.026). In contrast, when adding methylation status of genes not involved in the Ras pathway such as MGMT or CHFR, survival differences were not observed (MGMT: log-rank test, P=0.220; CHFR: log-rank test, P=0.118).

The survival impact of the KRAS variant combined with KRAS mutation status is independent of other prognostic factors. In multivariate analyses, statistically significant differences in cause-specific survival were not found for early-stage (HR 0.46; 95% CI: 0.18-1.14), stage III (HR 0.98, 95% CI: 0.55-1.74) or stage IV cases (HR 0.42; 95% CI: 0.17-1.06) with the G-allele (KRAS variant) as compared with wild types, although early-stage and stage IV G-allele (KRAS variant) carriers demonstrated an improved survival (Table 29).

TABLE 29
HRs and 95% CI for cause-specific mortality and clinicopathologic parameters and the
KRAS variant in 734 CRC cases from the Netherlands Cohort Study on diet and cancer.

| | | Early stage | | |
|---|---|---|---|---|
| | | (stage I and II) CRC | Stage III CRC | Stage IV CRC |
| KRAS-LCS6 variant | | 0.46 (0.18-1.14) | 0.98 (0.55-1.74) | 0.42 (0.17-1.06) |
| KRAS-LCS6 variant without | | 0.77 (0.30-1.97) | 0.95 (0.44-2.05) | 0.35 (0.11-1.13) |
| KRAS mutations | | | | |
| KRAS-LCS6 variant with | | No CRC-related deaths | 1.52 (0.66-3.54) | 0.60 (0.19-1.91) |
| KRAS mutations | | | | |
| Sex (male) | | 0.97 (0.60-1.57) | 0.92 (0.59-1.45) | 0.85 (0.44-1.64) |
| Age at diagnosis | | 0.99 (0.94-1.05) | 1.01 (0.96-1.06) | 1.02 (0.93-1.10) |
| Grade | 1 | 1 (reference) | 1 (reference) | 1 (reference) |
| | 2 | 1.40 (0.51-5.70) | 0.91 (0.34-2.45) | 2.14 (0.28-16.38) |
| | 3 | 0.77 (0.09-6.72) | 1.90 (0.52-6.94) | 14.47 (1.25-167.07) |
| | 4 | — | 4.17 (0.72-24.05) | 62.36 (2.11-1837.24) |
| Sublocation of the | Proximal | 1 (reference) | 1 (reference) | 1 (reference) |
| tumor | Distal | 0.76 (0.41-1.43) | 0.67 (0.37-1.19) | 0.55 (0.24-1.24) |
| | Rectosigmoid | 0.32 (0.11-0.94) | 0.60 (0.24-1.48) | 0.95 (0.27-3.35) |
| | Rectum | 0.49 (0.18-1.36) | 0.24 (0.08-0.69) | 0.35 (0.06-1.87) |

Early-stage G-allele (KRAS variant) carriers with KRAS mutations have a good prognosis; because none of these patients died due to CRC. In contrast, statistically significant differences in survival were not found between KRAS nonmutated early-stage (HR 0.77; 95% CI: 0.30-1.97), stage III (HR 0.95; 95% CI: 0.44-2.05) or stage IV cases (HR 0.35; 95% CI: 0.11-1.13) with the KRAS variant. However, stage III G-allele (KRAS variant) carriers with KRAS mutations presented a poor prognosis (HR 1.52; 95% CI: 0.66-3.54) although the comparison was not statistically significant. Because Dutch guidelines did not advise adjuvant treatment at the time patients were diagnosed with CRC in the NLCS, the proportion of patients that received adjuvant treatment was very low. Within the early-stage cases, 9% received adjuvant chemotherapy. With respect to more advanced stage, 31% of stage III and 19% of stage IV patients received adjuvant chemotherapy. Exclusion of adjuvant chemotherapy-treated patients did not alter our conclusions. In fact, exclusion of adjuvant chemotherapy-treated patients enhanced the difference between early-stage and stage III G-allele (KRAS variant) carriers with KRAS mutations (early stage: no CRC-related deaths; stage III: HR 2.36 95% CI: 0.99-5.67), implying that stage III G-allele (KRAS variant) carriers have a worse natural course of the disease. However, this analysis is based on small patient numbers.

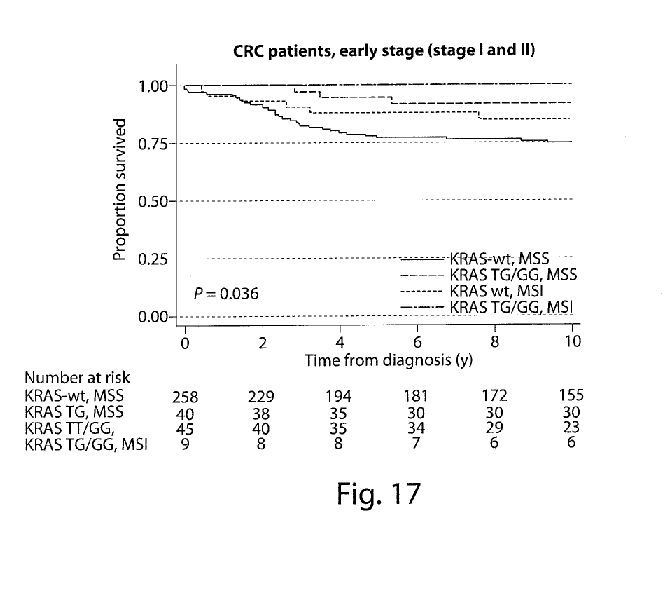

The survival impact of the KRAS variant is independent of microsatellite instability (MSI). Prior to the development of the biomarkers and methods provided herein, MSI was the only established molecular prognostic marker in CRC. Therefore, the effect of KRAS variant genotype was studied in patient populations stratified for MSI. Exclusion of patients that had an MSI tumor, which is associated with a good prognosis, did not alter the conclusions provided herein; both MSI and MSS cases with the KRAS variant had a good prognosis. In contrast, patients with the KRAS wild type had a poor prognosis, even if they had an MSI tumor (log-rank test, P=0.036) (FIG. 17). Additional analyses stratified for sex, tumor sublocation or differentiation grade within MSI patients were not possible due to limited patient numbers.

The risk of advanced stage CRC is not associated with the KRAS variant. To study the possibility that the KRAS variant allele predisposes for advanced stage CRC, the association between KRAS genotype and CRC risk was studied. The KRAS variant (G-allele) was found in 18% of the subcohort members. For CRC, a decreased risk of developing early-stage (stage I or II) CRC was found when carrying the KRAS variant (G-allele) (RR 0.68, 95% CI: 0.49-0.94). The risk of developing advanced stage CRC (stage III or IV) was not influenced by the KRAS-genotype (RR stage III: 1.02, 95% CI: 0.68-1.53; RR stage IV: 1.15, 95% CI: 0.63-2.09).

### Example 5

### The KRAS Variant, Patient Outcome in Metastatic Colorectal Cancer, and Response to Treatment

### Materials and Methods

Patient characteristics. A total of 559 mCRC patients, 300 treated in the University Hospital of Leuven with anti-EGFR moAb monotherapy and MoAb in combination with chemotherapy, as well as 148 patients from the Universite Paris Descartes treated with cetuximab-based salvage combination chemotherapy (De R W, et al. Lancet Oncol 2010; 11(8):753-762), and 111 previously published (Zhang W, al. Ann Oncol 2011; 22(1):104-109) mCRC patients treated with cetuximab monotherapy after failing fluoropyrimidine, irinotecan and oxaliplatin containing regimens (Zhang W, al. Ann Oncol 2011; 22(1):104-109; Lenz H J, et al. J Clin Oncol 2006; 24(30):4914-4921) had tissue available and amenable for analysis of the KRAS variant polymorphism. The mutational status of the KRAS and BRAF genes in the above mentioned patient populations is publicly available (De R W, et al. Lancet Oncol 2010; 11(8):753-762; Zhang W, al. Ann Oncol 2011; 22(1):104-109). The above mentioned molecular characteristics were correlated with ORR, PFS and OS. From the 559 mCRC patients entered in the study the KRAS 3′-UTR LCS6 variant was determined in 512 due to exhaustion of available DNA from other molecular testing.

Genetic analyses. Formalin-fixed, paraffin-embedded normal tissue from the patients' specimens was macroscopically dissected using a scalpel blade and DNA was isolated as previously described (De R W, et al. Lancet Oncol 2010; 11(8):753-762; Zhang W, al. Ann Oncol 2011; 22(1):104-109). DNA was amplified using, as previously described (Hollestelle A, et al. Breast Cancer Res Treat 2010), a custom-made Taqman genotyping assay (Applied Biosystems, Foster City, Calif.) designed specifically to identify the T or variant G allele of the KRAS-variant (rs61764370) with the forward primer: 5′-GCCAGGCTGGTCTCGAA-3′ (SEQ ID NO: 28), reverse primer: 5′-CTGAATAAATGAGTTCTGCAAAACAGGT T-3′(SEQ ID NO: 29), VIC reporter probe: 5′-CTCAAGTGATTCACCCA C-3′ (SEQ ID NO: 30), and FAM reporter probe: 5′-CAAGTGATTCACCCAC-3′ (SEQ ID NO: 31). The KRAS and BRAF mutational status was determined as previously described (De R W, et al. Lancet Oncol 2010; 11(8):753-762; Zhang W, al. Ann Oncol 2011; 22(1):104-109).

Cell line studies. A cell line with the KRAS variant (G-allele) (HCC2998) and a cell line without the allele and without a KRAS tumor acquired mutation (HT-29) were studied to evaluate the impact of treatment with chemotherapy alone or in combination with Cetuximab. Cell lines were treated with Cetuximab (100 nM) or none and dilutions of Irinotecan (1 mg/ml-100 mg/ml). Cells were plated, treated with agents 24 hours after plating, media was changed after a 24 hour exposure, and then survival was scored 48 hours later using the MTT assay.

Statistical analyses. The distribution of genotypes was tested for Hardy-Weinberg Equilibrium and the χ² test was p=0.8. Because of the low frequency of homozygotes for the KRAS variant allele, patient samples that were either heterozygous (TG) or homozygous (GG) for the KRAS variant allele were considered positive for the LCS6 (KRAS-variant or G allele) and entered the analyses as one group of at least one KRAS variant (G allele) genotypes. PFS and OS were measured as previously described (De R W, et al. Lancet Oncol 2010; 11(8):753-762; Zhang W, al. Ann Oncol 2011; 22(1):104-109)

The two-tailed Fisher's exact test was used to compare proportions between carriers of the wild-type (wt) TT genotype and carriers of at least one G allele genotypes (TG and GG). PFS and OS were estimated with the use of the Kaplan-Meier method and their association with genotypes was tested with the use of the log-rank test. The association of genotypes with objective response was determined by contingency table and the Fisher's exact test. To fully explore the possible influence of the KRAS variant, analyses were performed in the whole mCRC population, in the patients harboring no mutations in the KRAS and BRAF genes (double wt population) and in the KRAS variant population. The level of significance was set at a two-sided p value of <0.05. All statistical tests were performed using the statistical package SPSS version 13.

### Results

KRAS LCS6 in the entire patient cohort. In these 512 mCRC patients there were 403 carriers of the wt LCS6 TT genotype (72%), 102 (18%) carriers of the heterozygous KRAS variant TG allele and 7 (1.3%) of the homozygous KRAS variant GG allele, thus 109 (19.5%) carriers of at least one G allele genotype. KRAS mutations in codons 12, 13 and 61 were found in 184 patients (33%) and the BRAF V600E was found in 29 patients (5.3%). All patients had received anti-EGFR moAbs-based salvage treatment, 169 as monotherapy and 377 in combination with chemotherapy. No statistically significant differences were found between KRAS wt and KRAS variant carriers for sex and age at diagnosis. The characteristics of the 559 patients have been previously published (De R W, et al. Lancet Oncol 2010; 11(8):753-762; Zhang W, al. Ann Oncol 2011; 22(1):104-109).

As shown in Table 30 the distribution of the KRAS genotypes was different among patients harboring KRAS and BRAF mutations. In particular, whereas the percentage of at least one G variant allele genotype was equally distributed among the KRAS wt and mutant groups (20% in each), the KRAS variant (G allele) was twice as frequent in the BRAF V600E mutated group (40%) compared to the wt one (20%), resulting in a statistically significant difference (Fisher's exact test p=0.030).

TABLE 30
Distribution of the KRAS 3′-UTR LCS6 genotypes according to
KRAS and BRAF mutational status in the mCRC patients' cohort.

| | | | KRAS 3′-UTR LCS6 genotypes | | |
|---|---|---|---|---|---|
| | | | | | p value |
| | | | TT | | (fisher's |
| | Patients' population | | No of | TG + GG | exact |
| Feature | (No of patients) | | patients | No of patients | test) |
| KRAS | n = 484 | Mutant (n = 174) | 138 | 36 | 0.818 |
| status | | WT (n = 310) | 242 | 68 | |
| BRAF | n = 504 | Mutant (n = 28) | 17 | 11 | 0.030 |
| status | | WT (n = 476) | 379 | 97 | |

Abbreviations:
3′-UTR LCS6, 3′ untranslated region of the Let-7 complementary site,
WT, wild type.

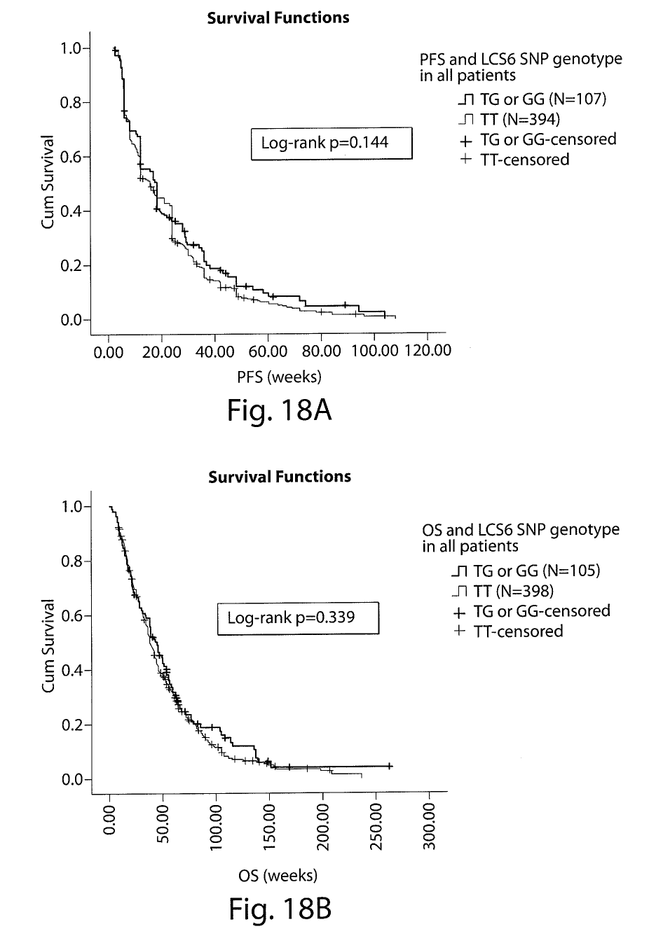

Outcome and Survival analysis in the entire patient cohort. In the cohort as a whole, for patients with PFS and OS information and LCS6 genotyping (n=510 and 503, respectively) no significant differences were detected regarding median PFS and OS between the LCS6 wt TT genotype carriers and the LCS6 G variant (KRAS variant) genotype carriers (FIGS. 18A and 18B). Similarly, no differences in PFS and OS were observed in the double (KRAS and BRAF) wt or in the KRAS variant patient cohort. Furthermore, no significant correlations regarding response (n=483) and skin rash (n=359) were observed between the KRAS variant and wt carriers in the whole and in the double wt patients' cohorts (Table 31).

TABLE 31
Outcome and survival analysis according to KRAS genotypes
and other clinical variables for the entire population.

| | | KRAS 3′-UTR LCS6 genotypes | | |
|---|---|---|---|---|
| Population cohort | Variables | TT | TG + GG | p value |
| All patients | median PFS (weeks) | 16 (14.3-17.6) | 18 (12.8-23.1) | 0.144 |
| | (95% CI) | | | (log-rank test) |
| | median OS (weeks) | 38 (34.74-41.26) | 45 (36.01-53.98) | 0.339 |
| | (95% CI) | | | (log-rank test) |
| Double (KRAS | median PFS (weeks) | 23.3 | 25.3 | 0.13 |
| and BRAF) wt | | | | (log-rank test) |
| patients | median OS (weeks) | 46 | 54 | 0.256 |
| | | | | (log-rank test) |
| KRAS mutated | median PFS (weeks) | 11 | 12 | 0.834 |
| patients | | | | (log-rank test) |
| | median OS (weeks) | 28 | 33 | 0.496 |
| | | | | (log-rank test) |
| All patients | responders (n) | 79 | 29 | 0.142 |
| | (CR + PR) | | | (Fisher's exact |
| | non-responders (n) | 301 | 74 | test) |
| | (SD + PD) | | | |
| Double (KRAS | responders (n) | 72 | 25 | 0.165 |
| and BRAF) wt | (CR + PR) | | | (Fisher's exact |
| patients | non-responders (n) | 141 | 32 | test) |
| | (SD + PD) | | | |
| All patients | skin rash | 149 | 48 | 0.2 |
| | (no/grade 1, n) | | | (Fisher's exact |
| | skin rash | 132 | 30 | test) |
| | (grade 2/3, n) | | | |
| Double (KRAS | skin rash | 71 | 23 | 0.149 |
| and BRAF) wt | (no/grade 1, n) | | | (Fisher's exact |
| patients | skin rash | 81 | 15 | test) |
| | (grade 2/3, n) | | | |

Abbreviations: 3′-UTR LCS6, 3′ untranslated region of the Let-7 complementary site; WT, wild type; PFS, progression-free survival; OS, overall survival; CR, complete response; PR, partial response; SD, stable disease; PD, progressive disease.

Progression free survival analysis correlated with treatment. Patients who received moAbs monotherapy and moAbs combination therapy were analyzed separately. From the 501 patients evaluable for LCS6 SNP genotyping and treatment administration, 160 (32%) received anti-EGFR moAbs as monotherapy. Of the monotherapy patients, 128 (80%) were carriers of the LCS6 wt TT genotype and 32 (20%) were carriers of the LCS6 G variant genotype. There were 341 (68%) patients who received multiple chemotherapy combinations. Of the combination treatment patients, 266 (78%) were carriers of the LCS6 wt TT genotype and 75 (22%) were carriers of the LCS6 at least one G variant genotype.

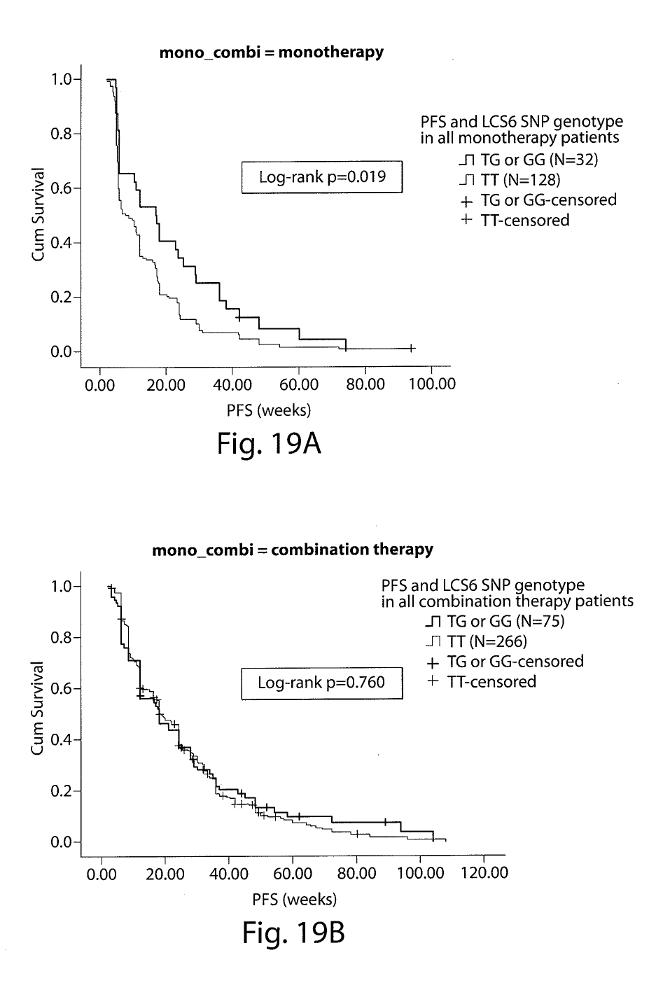

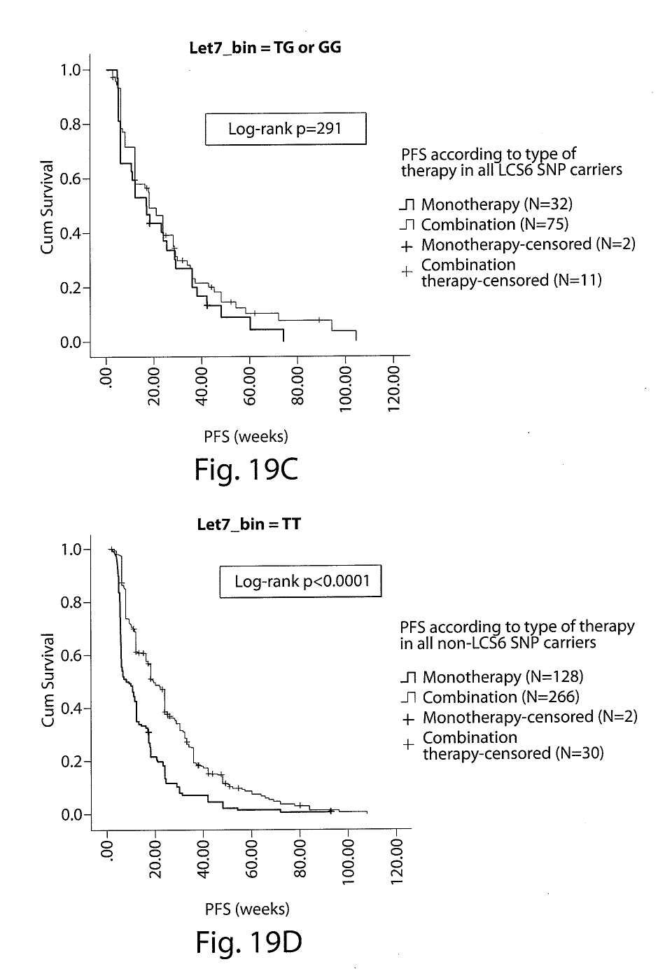

The median PFS of the whole monotherapy patients' population was 10.43 weeks (95% CI: 7.73-13.12 weeks) and a statistically significant difference (p=0.019, log-rank test) was observed between the LCS6 wt TT genotype carriers, 7.85 weeks (95% CI: 3.897-11.817 weeks), and the LCS6 G variant (KRAS variant) genotype carriers, 16.86 weeks (95% CI: 10.2-23.51 weeks) (FIG. 19A). The median PFS of the whole combination therapy patients' population was 18 weeks (95% CI: 15.87-20.12 weeks) and no statistically significant difference (p=0.760, log-rank test) was observed between the LCS6 wt TT genotype carriers, 18.43 weeks (95% CI: 16.16-20.69 weeks), and the LCS6 G variant genotype carriers, 18 weeks (95% CI: 9.97-26.02 weeks) (FIG. 19B). There was also no significant difference (p=0.291, log-rank test) between PFS for KRAS variant patients that received moAbs therapy [16.86 weeks, (95% CI: 8.55-25.18 weeks)] versus combination therapy [18 weeks, (95% CI: 13.37-22.64 weeks)] (FIG. 19C), while there was a significant benefit with the addition of chemotherapy for non-KRAS variant patients [p<0.0001, log-rank test, PFS for moAbs monotherapy 7.86 weeks, (95% CI: 3.9-11.82 weeks) versus combination therapy 19.29 weeks, (95% CI: 17-21.58 weeks) (FIG. 19D). Of note, there was no significant difference in PFS between KRAS variant patients treated with monotherapy therapy versus non-KRAS variant patients treated with combination therapy.

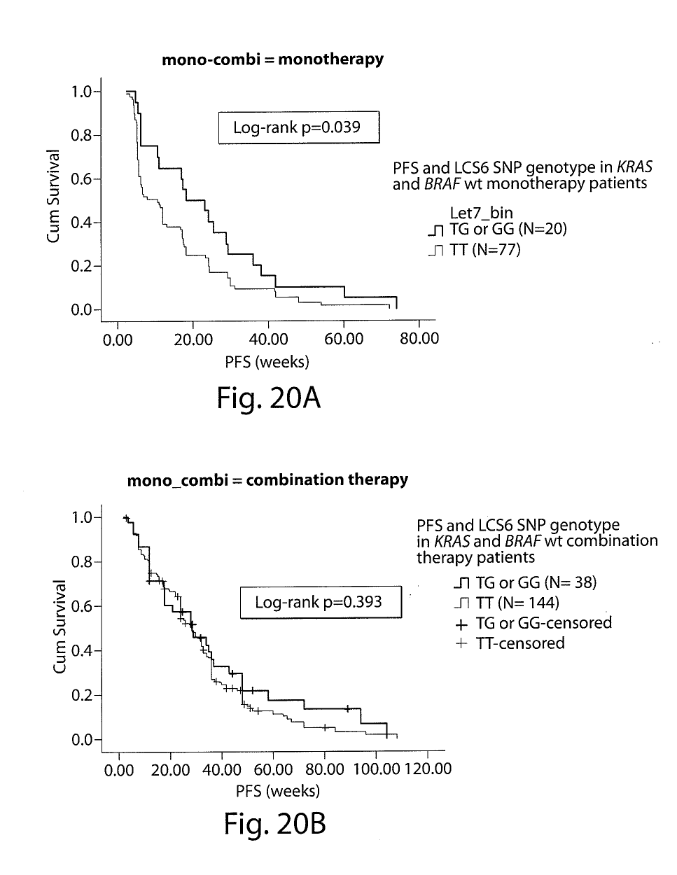

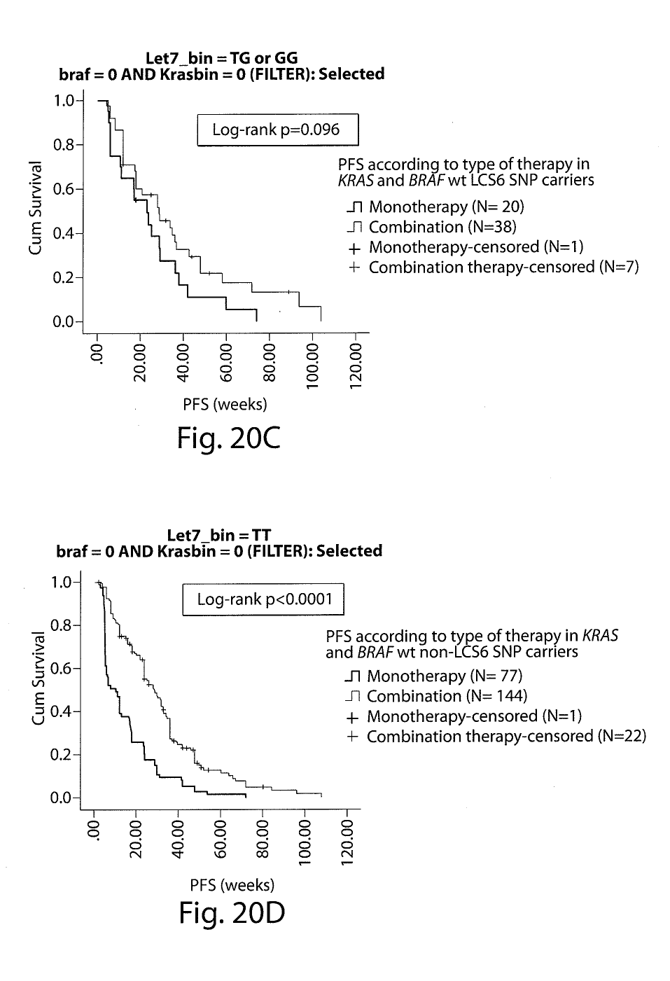

In the double (KRAS and BRAF) wt patients' population the median PFS of the monotherapy patients was 12 weeks (95% CI: 8.38-15.61 weeks) and a statistically significant difference (p=0.039, log-rank test) was again observed between the LCS6 wt TT genotype carriers, 10.43 weeks (95% CI: 6.74-14.11 weeks), and the LCS6 G variant genotype carriers, 18 weeks (95% CI: 5.16-30.83 weeks) (FIG. 20A). In the double wt patients' population the median PFS of the combination therapy patients was 28.71 weeks (95% CI: 24.98-32.43 weeks) and no statistically significant difference (p=0.39, log-rank test) was observed between the LCS6 wt TT genotype carriers, 28.3 weeks (95% CI: 24.15-32.45 weeks), and the LCS6 G variant genotype carriers, 28.85 weeks (95% CI: 14.82-42.87 weeks) (FIG. 20B). There was no significant improvement (p=0.096, log-rank test) between PFS for LCS6 variant patients that received moAbs monotherapy [23 weeks, (95% CI: 9.5-36.5 weeks)] versus combination therapy [28 weeks, (95% CI: 14.83-42.87 weeks)] (FIG. 20C), while there was for non-LCS6 patients [p<0.0001, log-rank test, PFS for moAbs monotherapy 10.43 weeks, (95% CI: 6.75-14.15 weeks) versus combination therapy 28.71 weeks, (95% CI: 24.8-32.6 weeks) (FIG. 20D). There was no difference in PFS between KRAS variant (G allele) patients receiving moAbs monotherapy and non-KRAS variant patients receiving combination therapy.

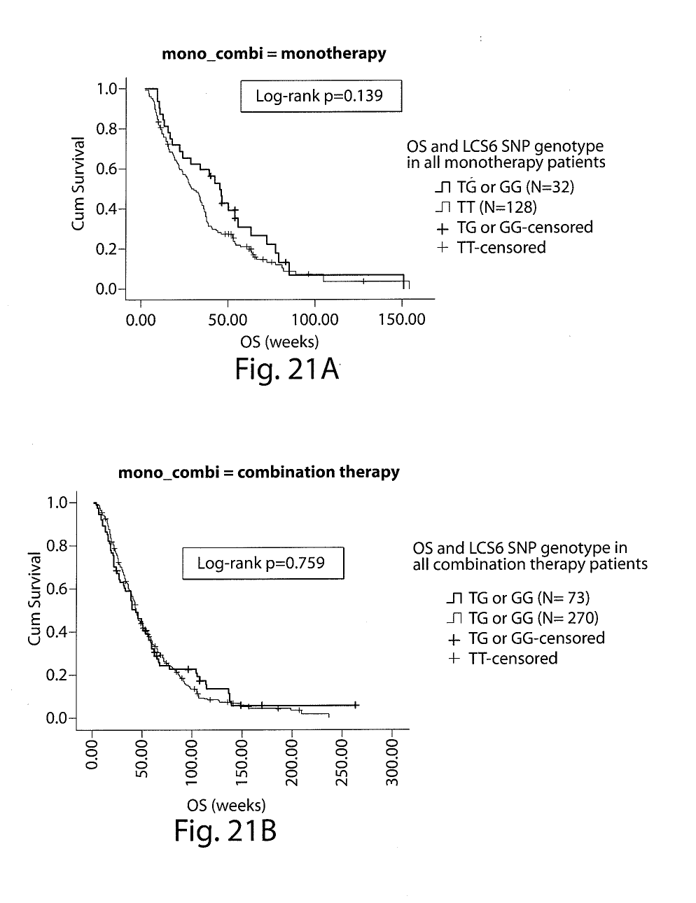

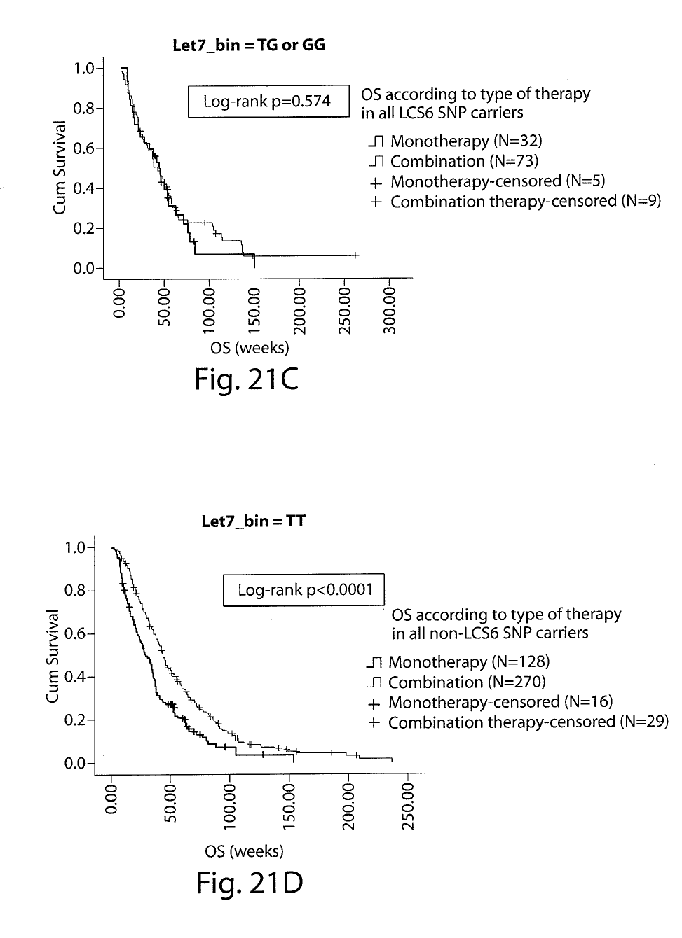

Overall survival analysis correlated with treatment. The median OS of the whole monotherapy patients' population was 33.14 weeks (95% CI: 26.70-39.57 weeks) and no statistically significant difference (p=0.139, log-rank test) was observed between the LCS6 wt TT genotype carriers, 28.85 weeks (95% CI: 22.53-35.18 weeks), and the LCS6 G variant genotype carriers, 45 weeks (95% CI: 35.02-54.97 weeks) (FIG. 21A). The median OS of the whole combination therapy patients' population was 44 weeks (95% CI: 40.11-47.88 weeks) and no statistically significant difference (p=0.759, log-rank test) was observed between the LCS6 wt TT genotype carriers, 44 weeks (95% CI: 40.06-47.93 weeks), and the LCS6 at least one G variant genotype carriers, 43 weeks (95% CI: 29.8-56.2 weeks) (FIG. 21B). Again, there was no significant improvement (p=0.574, log-rank test) between OS for KRAS variant patients that received moAbs monotherapy [45 weeks, (95% CI: 35-55 weeks)] versus combination therapy [43 weeks, (95% CI: 29.8-56.2 weeks)] (FIG. 21C), while there was a benefit of chemotherapy addition for non-KRAS variant patients [p<0.0001, log-rank test, OS for moAbs monotherapy 28.86 weeks, (95% CI: 22.53-35.18 weeks) versus combination therapy 44 weeks, (95% CI: 40-47.93 weeks) (FIG. 21D). Again, there was no significant difference in OS between LCS6 G variant carriers treated with monotherapy, and non-KRAS variant carriers treated with combination therapy.

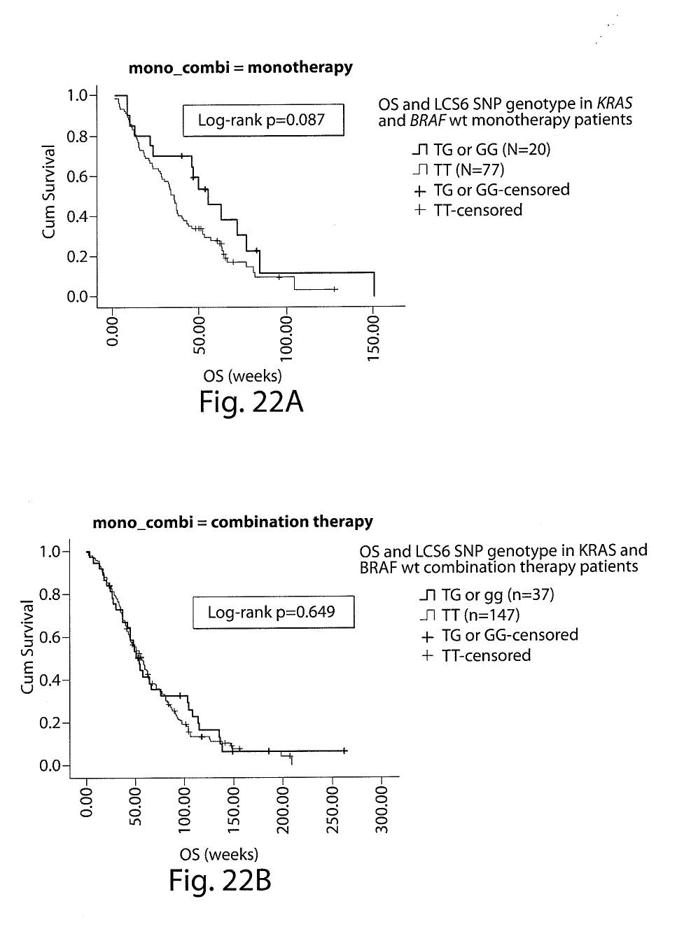

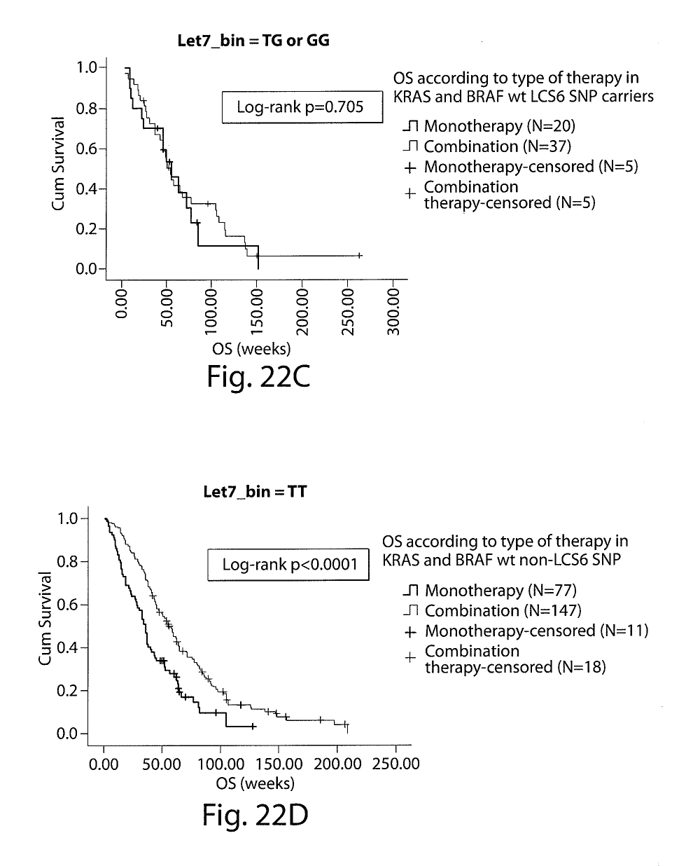

In the double (KRAS and BRAF) wt patients' population the median OS of the monotherapy patients was 37 weeks (95% CI: 30.82-43.17 weeks) and a trend towards a statistically significant difference (p=0.087, log-rank test) was observed between the LCS6 wt TT genotype carriers, 35.71 weeks (95% CI: 32.03-39.4 weeks), and the LCS6 at least one G variant genotype carriers, 55.43 weeks (95% CI: 36.98-73.87 weeks) (FIG. 22A). In the double wt patients' population, the median OS of the combination therapy patients was 55 weeks (95% CI: 48.3-61.7 weeks) and no statistically significant difference (p=0.649, log-rank test) was observed between the LCS6 wt TT genotype carriers, 57 weeks (95% CI: 49.4-64.6 weeks), and the LCS6 at least one G variant genotype carriers, 54 weeks (95% CI: 45.46-62.53 weeks) (FIG. 22B). There was no significant improvement (p=0.705, log-rank test) between OS for KRAS variant (G allele) patients that received moAbs monotherapy [55.43 weeks, (95% CI: 37-73.87 weeks)] versus combination therapy [54 weeks, (95% CI: 45.47-62.54 weeks)] (FIG. 22C), while there was for non-KRAS variant patients [p<0.0001, log-rank test, OS for moAbs monotherapy 35.71 weeks, (95% CI: 32-39.4 weeks) versus combination therapy 57 weeks, (95% CI: 49.4-64.6 weeks) (FIG. 22D). There was no significant difference between double wild-type patients KRAS variant carriers treated with monotherapy versus non-LCS6 carriers treated with combination therapy.

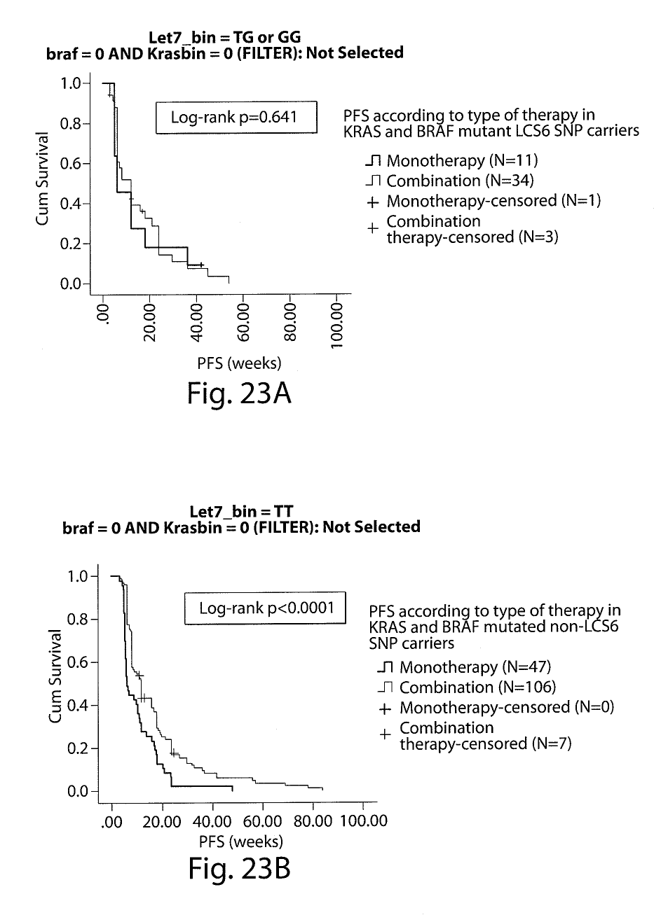

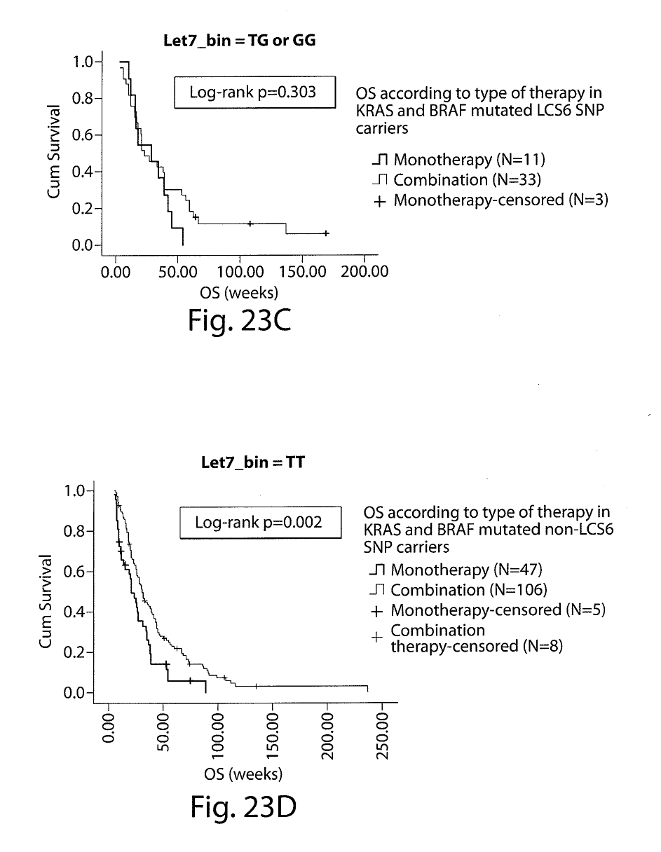

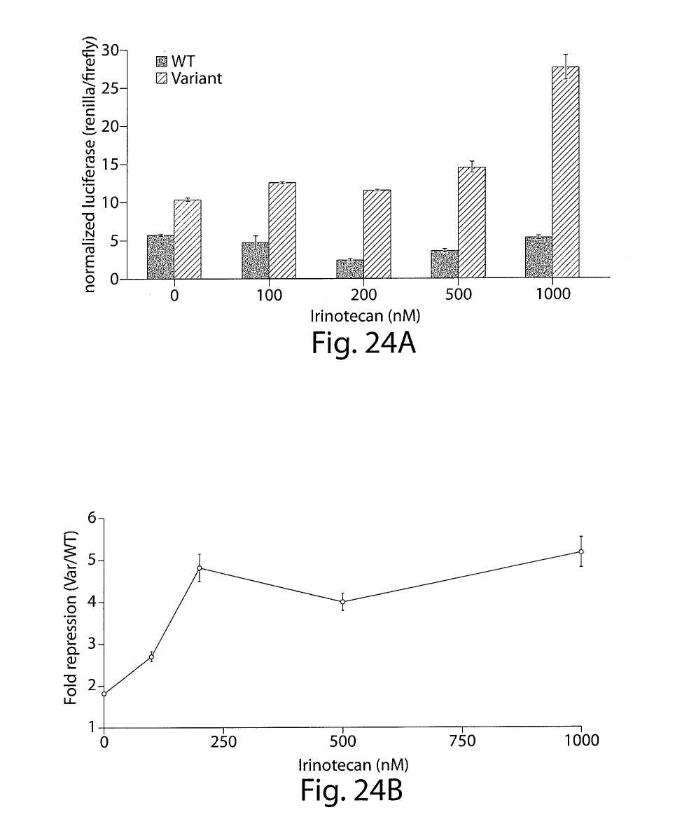

The LCS6 variant is prognostic in KRAS and BRAF mutated patients. In the KRAS and BRAF mutated patients' population no statistical significant differences regarding PFS and OS were observed in patients treated with both anti-EGFR moAbs monotherapy and in combination with chemotherapy (data not shown). Median PFS times were identical between KRAS variant and non-KRAS variant patients, with no significant improvement (p=0.641, log-rank test) between PFS for KRAS variant patients that received moAbs monotherapy [6 weeks, (95% CI: 0-13.25 weeks)] versus combination therapy [12 weeks, (95% CI: 6.45-17.56 weeks)] (FIG. 23A). There was a significant improvement in PFS for non-KRAS variant patients [p<0.0001, log-rank test, PFS for moAbs monotherapy 6 weeks, (95% CI: 4.46-7.53 weeks) versus combination therapy 12 weeks, (95% CI: 9.72-14.28 weeks) (FIG. 23B). For OS, there was no significant difference (p=0.303, log-rank test) between OS for KRAS variant (G allele) patients that received moAbs monotherapy [28.43 weeks, (95% CI: 9.47-47.39 weeks)] versus combination therapy [23 weeks, (95% CI: 10.8-35.19 weeks)] (FIG. 23C), while there was for non-KRAS variant patients [p=0.002, log-rank test, OS for moAbs monotherapy 21.29 weeks, (95% CI: 15-27.55 weeks) versus combination therapy 31 weeks, (95% CI: 25.65-36.34 weeks) (FIG. 23D).

The KRAS variant and response. From the whole population of 483 patients that were evaluable for both response and KRAS variant genotyping, 147 (30.4%) had received anti-EGFR moAbs as monotherapy and 336 (69.6%) with multiple chemotherapy combinations. In the monotherapy group 123 (83.6%) patients were non-responders (SD and PD), 104 LCS6 wt and 19 LCS6 variant (KRAS variant) carriers, and 24 (16.4%) were responders (PR and CR), 13 LCS6 wt and 11 LCS6 variant (KRAS variant) carriers. A statistically significant difference was observed between the wt and KRAS variant genotype carriers distribution in the responders and non-responders groups (Fisher's exact test p=0.002). In the combination with chemotherapy group 252 (75%) patients were non-responders (SD and PD) and 84 (25%) were responders (PR and CR). No statistically significant difference was observed between the wt and KRAS variant genotype carriers, 197 vs. 55 non-responders and 66 vs. 18 responders, respectively (Fisher's exact test p=1).

In the 270 double (KRAS and BRAF) wt population 90 (33.3%) had received anti-EGFR moAbs as monotherapy and 180 (66.6%) with multiple chemotherapy combinations. In the monotherapy group 71 (78.8%) patients were non-responders (SD and PD), 60 LCS6 wt and 11 LCS6 variant (KRAS variant) carriers and 19 (21.2%) were responders (PR and CR), 10 LCS6 wt and 9 LCS6 variant (KRAS variant) carriers. A statistically significant difference was observed between the wt and KRAS variant genotype carriers distribution in the responders and non-responders groups (Fisher's exact test p=0.010). In the combination with chemotherapy group 102 (56.6%) patients were non-responders (SD and PD) and 78 (43.4%) were responders (PR and CR). No statistically significant difference was observed between the wt and KRAS variant genotype carriers, 81 vs. 21 non-responders and 62 vs. 16 responders, respectively (Fisher's exact test p=1).

Cell line studies of the effect of moAbs monotherapy and combination therapy and the LCS6 variant. To confirm that the KRAS variant (G allele) predicts response to moAbs monotherapy, without any benefit of additional cytotoxic therapy, the impact of monotherapy versus combination therapy in colon cancer cell lines with and without the LCS6 G variant was evaluated. It was discovered that in non-KRAS variant cell lines, the addition of Cetuximab to cytotoxic therapy, both radiation as well as irinotecan chemotherapy, increased cell death as compared to cytotoxic therapy alone. In contrast, in a cell line with the KRAS variant (G allele), there was no additional cell kill with the addition of Cetuximab to cytotoxic therapy, and in the case of radiation in fact higher cell survival when Cetuximab was added. These findings are consistent with our in vivo findings, that there is no benefit of the combination of Cetuximab with cytotoxic therapy in KRAS variant (G allele) carriers.

### Other Embodiments

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this disclosure has been particularly shown and described with references to preferred embodiments thereof, it will be understood by those skilled in the art that various changes in form and details may be made therein without departing from the scope of the disclosure encompassed by the appended claims.

What is claimed is:

1. A method of identifying a subject or patient at risk for developing an estrogen receptor (ER) and progesterone receptor (PR) negative (ER/PR negative) breast cancer, comprising detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of the mutation indicates an increased risk of developing the ER/PR negative breast cancer in the subject.

2. A method of predicting the onset of developing an estrogen receptor (ER) and progesterone receptor (PR) negative (ER/PR negative) breast cancer in a subject or patient at risk for developing breast cancer, comprising detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of the mutation indicates an earlier onset of developing the ER/PR negative breast cancer.

3. The method of claim 2, wherein the ER/PR negative breast cancer is also negative for HER2, and therefore, is a triple negative breast cancer (TNBC).

4. The method of claim 3, wherein the triple negative breast cancer (TNBC) is a basal or luminal tumor.

5. The method of claim 4, wherein the triple negative breast cancer (TNBC) is a basal tumor that expresses a transcript or protein encoded by the epidermal growth factor receptor (EGFR) or the cytokeratin 5/6 (CK5/6) gene.

6. The method of claim 1, 2, or 3, wherein the breast cancer is further characterized by low or negative expression of the breast cancer 1 (BRCA1) gene.

7. The method of claim 1, 2, or 3, wherein the subject or patient is pre-menopausal.

8. The method of claim 1, 2, or 3, wherein the subject or patient is 51 years of age or younger.

9. A method of prognosing a subject or patient with epithelial ovarian cancer (EOC), comprising detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of the mutation indicates a decreased survival rate when compared to a control.

10. The method of claim 9, wherein the subject or patient is post-menopausal, 52 years of age, or at least 52 years of age.

11. The method of claim 9, wherein the control does not carry the mutation.

12. The method of claim 1, wherein the survival rate is overall survival, five-year survival or one-year survival.

13. A method of predicting the response of an epithelial ovarian cancer (EOC) cell to a platinum-based chemotherapy, comprising detecting a mutation in let-7 complementary site LCS6 of human KRAS in a patient sample, wherein the mutation is a SNP comprising a uracil (U) or thymine (T) to guanine (G) transition at position 4 of LCS6, wherein the presence of the mutation indicates a resistance to platinum-based chemotherapy.

14. The method of claim 13, wherein the EOC cell is evaluated in vitro or ex vivo.

15. The method of claim 14, wherein the EOC cell is evaluated ex vivo from a subject who is post-menopausal, 52 years of age, or at least 52 years of age.

16. The method of claim 14, wherein the EOC cell is evaluated in vitro and wherein the EOC cell is isolated, reproduced, or derived from the BG1, CAOV3, or IGR-OV1 cell line.

17. The method of claim 13, wherein the platinum-based chemotherapy is carboplatin or paclitaxel.

18. The method of claim 13, wherein the platinum-based chemotherapy is an adjuvant therapy.

19. A method of prognosing a subject or patient with colorectal cancer (CRC), comprising detecting a mutation in let-7 complementary site LCS6 of human KRAS in a patient sample, wherein the mutation is a SNP comprising a uracil (U) or thymine (T) to guanine (G) transition at position 4 of LCS6, wherein the presence of the mutation indicates a increased survival rate when compared to a control.

20. The method of claim 19, wherein the detecting step further comprises microsatellite-instability (MSI) analysis.

21. The method of claim 19, wherein the colorectal cancer (CRC) is early stage CRC.

22. The method of claim 19, wherein the colorectal cancer (CRC) is stage 1 or 2 CRC.

23. The method of claim 19, wherein the control does not carry the KRAS-variant.

24. The method of claim 23, wherein the control has a second mutation in the KRAS gene.

25. The method of claim 19, wherein the subject or patient has a second mutation in the KRAS gene.

26. The method of claim 19, wherein the subject or control carries one or more mutations in the BRAF gene.

27. The method of claim 19, wherein the subject or control has a hypermethylated RASSF1A promoter.

28. The method of claim 19, wherein the survival rate is overall survival, five-year survival or one-year survival.

29. A method of predicting the response of a cancer cell to a monoclonal antibody monotherapy, comprising detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of the mutation indicates a sensitivity to monoclonal antibody monotherapy.

30. The method of claim 29, wherein the cancer cell is a colorectal cancer (CRC) cell.

31. The method of claim 29, wherein the cancer cell is evaluated in vitro or ex vivo.

32. The method of claim 29, wherein the monoclonal antibody monotherapy is Cetuximab.

33. A method of predicting the response of a cancer cell to the combination of a chemotherapy and monoclonal antibody therapy, comprising detecting a mutation in let-7 complementary site LCS6 of human KRAS in a patient sample, wherein the mutation is a SNP comprising a uracil (U) or thymine (T) to guanine (G) transition at position 4 of LCS6, and wherein the presence of the mutation indicates a resistance to the combination.

34. The method of claim 33, wherein the cancer cell is a colorectal cancer (CRC) cell.

35. The method of claim 33, wherein the cancer cell is evaluated in vitro or ex vivo.

36. The method of claim 33, wherein the monoclonal antibody monotherapy is Cetuximab.

37. The method of claim 33, wherein the chemotherapy is a cytotoxic agent.

38. The method of claim 37, wherein the cytotoxic agent is irinotecan.

39. A method of predicting the an increased risk of vascularization of a tumor, comprising
(a) detecting a mutation in let-7 complementary site LCS6 of human KRAS in a first patient sample, wherein the mutation is a SNP comprising a uracil (U) or thymine (T) to guanine (G) transition at position 4 of LCS6, and
(b) determining the expression level of a miRNA selected from the group consisting of miR-23 and miR-27 in a second patient sample,
wherein the presence of the mutation in (a) and an increase in the expression level of a miRNA in (b) compared to a control indicates transcriptional silencing of an anti-angiogenic gene, thereby predicting the an increased risk of vascularization of the tumor.

40. The method of claim 39, wherein the anti-angiogenic gene is Sprouty2 or Sema 6A.

41. The method of claim 39 or 40, wherein the tumor comprises a cancer cell derived from a(n) AIDS-related cancer, breast cancer, cancer of the digestive/gastrointestinal tract, anal cancer, appendix cancer, bile duct cancer, colon cancer, colorectal cancer, esophageal cancer, gallbladder cancer, islet cell tumors, pancreatic neuroendocrine tumors, liver cancer, pancreatic cancer, rectal cancer, small intestine cancer, stomach (gastric) cancer, endocrine system cancer, adrenocortical carcinoma, parathyroid cancer, pheochromocytoma, pituitary tumor, thyroid cancer, eye cancer, intraocular melanoma, retinoblastoma, bladder cancer, kidney (renal cell) cancer, penile cancer, prostate cancer, transitional cell renal pelvis and ureter cancer, testicular cancer, urethral cancer, Wilms' tumor, other childhood kidney tumors, germ cell cancer, central nervous system cancer, extracranial germ cell tumor, extragonadal germ cell tumor, ovarian germ cell tumor, gynecologic cancer, cervical cancer, endometrial cancer, gestational trophoblastic tumor, ovarian epithelial cancer, uterine sarcoma, vaginal cancer, vulvar cancer, head and neck cancer, hypopharyngeal cancer, laryngeal cancer, lip and oral cavity cancer, metastatic squamous neck cancer with occult primary, mouth cancer, nasopharyngeal cancer, oropharyngeal cancer, paranasal sinus and nasal cavity cancer, pharyngeal cancer, salivary gland cancer, throat cancer, musculoskeletal cancer, bone cancer, Ewing's sarcoma, gastrointestinal stromal tumors (GIST), osteosarcoma, malignant fibrous histiocytoma of bone, rhabdomyosarcoma, soft tissue sarcoma, uterine sarcoma, neurologic cancer, brain tumor, astrocytoma, brain stem glioma, central nervous system atypical teratoid/rhabdoid tumor, central nervous system embryonal tumors, central nervous system germ cell tumor, craniopharyngioma, ependymoma, medulloblastoma, spinal cord tumor, supratentorial primitive neuroectodermal tumors and pineoblastoma, neuroblastoma, respiratory cancer, thoracic cancer, non-small cell lung cancer, small cell lung cancer, malignant mesothelioma, thymoma, thymic carcinoma, skin cancer, Kaposi's sarcoma, melanoma, or Merkel cell carcinoma.

42. The method of claim 39 or 41, wherein the tumor is metastic.

43. A method of predicting an increased survival or proliferation of a cancer cell under hypoxic conditions, comprising
(a) detecting a mutation in let-7 complementary site LCS6 of human KRAS in a first patient sample, wherein the mutation is a SNP comprising a uracil (U) or thymine (T) to guanine (G) transition at position 4 of LCS6, and
(b) determining the expression level of a miR-210 miRNA in a second patient sample,
wherein the presence of the mutation in (a) and an increase in the expression level of the miRNA in (b) compared to a control predicts an increased survival or proliferation of the cancer cell under hypoxic conditions.

44. The method of claim 43, wherein the cancer cell is derived from a(n) AIDS-related cancer, breast cancer, cancer of the digestive/gastrointestinal tract, anal cancer, appendix cancer, bile duct cancer, colon cancer, colorectal cancer, esophageal cancer, gallbladder cancer, islet cell tumors, pancreatic neuroendocrine tumors, liver cancer, pancreatic cancer, rectal cancer, small intestine cancer, stomach (gastric) cancer, endocrine system cancer, adrenocortical carcinoma, parathyroid cancer, pheochromocytoma, pituitary tumor, thyroid cancer, eye cancer, intraocular melanoma, retinoblastoma, bladder cancer, kidney (renal cell) cancer, penile cancer, prostate cancer, transitional cell renal pelvis and ureter cancer, testicular cancer, urethral cancer, Wilms' tumor, other childhood kidney tumors, germ cell cancer, central nervous system cancer, extracranial germ cell tumor, extragonadal germ cell tumor, ovarian germ cell tumor, gynecologic cancer, cervical cancer, endometrial cancer, gestational trophoblastic tumor, ovarian epithelial cancer, uterine sarcoma, vaginal cancer, vulvar cancer, head and neck cancer, hypopharyngeal cancer, laryngeal cancer, lip and oral cavity cancer, metastatic squamous neck cancer with occult primary, mouth cancer, nasopharyngeal cancer, oropharyngeal cancer, paranasal sinus and nasal cavity cancer, pharyngeal cancer, salivary gland cancer, throat cancer, musculoskeletal cancer, bone cancer, Ewing's sarcoma, gastrointestinal stromal tumors (GIST), osteosarcoma, malignant fibrous histiocytoma of bone, rhabdomyosarcoma, soft tissue sarcoma, uterine sarcoma, neurologic cancer, brain tumor, astrocytoma, brain stem glioma, central nervous system atypical teratoid/rhabdoid tumor, central nervous system embryonal tumors, central nervous system germ cell tumor, craniopharyngioma, ependymoma, medulloblastoma, spinal cord tumor, supratentorial primitive neuroectodermal tumors and pineoblastoma, neuroblastoma, respiratory cancer, thoracic cancer, non-small cell lung cancer, small cell lung cancer, malignant mesothelioma, thymoma, thymic carcinoma, skin cancer, Kaposi's sarcoma, melanoma, or Merkel cell carcinoma.

45. A method of predicting an increased survival or proliferation of a cancer cell, comprising
(a) detecting a mutation in let-7 complementary site LCS6 of human KRAS in a first patient sample, wherein the mutation is a SNP comprising a uracil (U) or thymine (T) to guanine (G) transition at position 4 of LCS6, and
(b) determining the methylation status of a promoter of a tumor suppressor gene in a second patient sample,
wherein the presence of the mutation in (a) and an increase in the methylation of a promoter (b) compared to a control predicts an increased survival or proliferation of the cancer cell.

46. The method of claim 45, wherein tumor suppressor gene is Notch1.

47. The method of claim 45, wherein the cancer cell is derived from a(n) AIDS-related cancer, breast cancer, cancer of the digestive/gastrointestinal tract, anal cancer, appendix cancer, bile duct cancer, colon cancer, colorectal cancer, esophageal cancer, gallbladder cancer, islet cell tumors, pancreatic neuroendocrine tumors, liver cancer, pancreatic cancer, rectal cancer, small intestine cancer, stomach (gastric) cancer, endocrine system cancer, adrenocortical carcinoma, parathyroid cancer, pheochromocytoma, pituitary tumor, thyroid cancer, eye cancer, intraocular melanoma, retinoblastoma, bladder cancer, kidney (renal cell) cancer, penile cancer, prostate cancer, transitional cell renal pelvis and ureter cancer, testicular cancer, urethral cancer, Wilms' tumor, other childhood kidney tumors, germ cell cancer, central nervous system cancer, extracranial germ cell tumor, extragonadal germ cell tumor, ovarian germ cell tumor, gynecologic cancer, cervical cancer, endometrial cancer, gestational trophoblastic tumor, ovarian epithelial cancer, uterine sarcoma, vaginal cancer, vulvar cancer, head and neck cancer, hypopharyngeal cancer, laryngeal cancer, lip and oral cavity cancer, metastatic squamous neck cancer with occult primary, mouth cancer, nasopharyngeal cancer, oropharyngeal cancer, paranasal sinus and nasal cavity cancer, pharyngeal cancer, salivary gland cancer, throat cancer, musculoskeletal cancer, bone cancer, Ewing's sarcoma, gastrointestinal stromal tumors (GIST), osteosarcoma, malignant fibrous histiocytoma of bone, rhabdomyosarcoma, soft tissue sarcoma, uterine sarcoma, neurologic cancer, brain tumor, astrocytoma, brain stem glioma, central nervous system atypical teratoid/rhabdoid tumor, central nervous system embryonal tumors, central nervous system germ cell tumor, craniopharyngioma, ependymoma, medulloblastoma, spinal cord tumor, supratentorial primitive neuroectodermal tumors and pineoblastoma, neuroblastoma, respiratory cancer, thoracic cancer, non-small cell lung cancer, small cell lung cancer, malignant mesothelioma, thymoma, thymic carcinoma, skin cancer, Kaposi's sarcoma, melanoma, or Merkel cell carcinoma.

48. The method of claim 45, wherein survival comprises maintaining tumorigenic potential.

49. The method of claim 45 or 48, wherein the cancer cell is a cancer stem cell.


SEQUENCE LISTING (31 sequences)

SEQ ID NO: 1 - DNA, 25 bp, Homo sapiens
gacaguggaaguuuuuuuuuccucg

SEQ ID NO: 2 - DNA, 19 bp, Homo sapiens
auuagugucaucuugccuc

SEQ ID NO: 3 - DNA, 25 bp, Homo sapiens
aaugcccuacaucuuauuuuccuca

SEQ ID NO: 4 - DNA, 24 bp, Homo sapiens
gguucaagcgauucucgugccucg

SEQ ID NO: 5 - DNA, 24 bp, Homo sapiens
ggcugguccgaacuccugaccuca

SEQ ID NO: 6 - DNA, 21 bp, Homo sapiens
gauucacccaccuuggccuca

SEQ ID NO: 7 - DNA, 28 bp, Homo sapiens
ggguguuaagacuugacacaguaccucg

SEQ ID NO: 8 - DNA, 28 bp, Homo sapiens
agugcuuaugaggggauauuuaggccuc

SEQ ID NO: 9 - DNA, 5436 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 10 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 11 - DNA, 5436 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgatgcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 12 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgatgcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 13 - DNA, 21 bp, Homo sapiens
gaugcacccaccuuggccuca

SEQ ID NO: 14 - DNA, 22 bp, Homo sapiens
uugauauguuggaugauggagu

SEQ ID NO: 15 - DNA, 22 bp, Homo sapiens
uugguguguuggaugauggagu

SEQ ID NO: 16 - DNA, 22 bp, Homo sapiens
uugguauguuggaugauggagu

SEQ ID NO: 17 - DNA, 21 bp, Homo sapiens
ugauacguuggaugauggaga

SEQ ID NO: 18 - DNA, 20 bp, Homo sapiens
uauauguuggaggauggagu

SEQ ID NO: 19 - DNA, 22 bp, Homo sapiens
uugauauguuagaugauggagu

SEQ ID NO: 20 - DNA, 20 bp, Homo sapiens
gacauguuugaugauggagu

SEQ ID NO: 21 - DNA, 21 bp, Homo sapiens
ugucguguuuguugauggagu

SEQ ID NO: 22 - DNA, 52 bp, Homo sapiens
gtctcgaactcctgacctcaagtgatgcacccaccttggcctcataaacctg

SEQ ID NO: 23 - RNA, 21 nt, Artificial Sequence
  siRNA sequence
ugcaucacuugaggucaggag

SEQ ID NO: 24 - RNA, 21 nt, Artificial Sequence
  siRNA sequence
ccugaccucaagugaugcacc

SEQ ID NO: 25 - DNA, 21 bp, Artificial Sequence
  siRNA sequence
tgcatcacuugaggucaggag

SEQ ID NO: 26 - RNA, 21 nt, Artificial Sequence
  siRNA sequence
ucaucacuugaggucaggagu

SEQ ID NO: 27 - RNA, 21 nt, Artificial Sequence
  siRNA sequence
uccugaccucaagugaugcac

SEQ ID NO: 28 - DNA, 17 bp, Artificial Sequence
  Primer sequence
gccaggctggtctcgaa

SEQ ID NO: 29 - DNA, 29 bp, Artificial Sequence
  Primer sequence
ctgaataaatgagttctgcaaaacaggtt

SEQ ID NO: 30 - DNA, 18 bp, Artificial Sequence
  Primer sequence
ctcaagtgattcacccac

SEQ ID NO: 31 - DNA, 16 bp, Artificial Sequence
  Primer sequence
caagtgattcacccac